SEQUENCE LISTING (2 sequences)

SEQ ID NO: 1 - DNA, 294 bp, Artificial Sequence
  The nucleotide sequence encoding Gold-binding Protein (GBP) is from the plasmid designated pSB3053 in Brown, Nature Biotechnology 15269-272 (1997)
atgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaactcaggcaaccagcgggactatccagagcatgcatggaaaaattcaggcaaccagcgggactatccagagc

SEQ ID NO: 2 - protein, 294 aa, Artificial Sequence
  The amino acid sequence of Gold-binding Protein is encoded by a nucleic acid sequence from the plasmid designated pSB3053 in Brown, Nature Biotechnology 15269-272 (1997)
METHISGLYLYSTHRGLNALATHRSERGLYTHRILEGLNSERMETHISGLYLYSTHRGLNALATHRSERGLYTHRILEGLNSERMETHISGLYLYSTHRGLNALATHRSERGLYTHRILEGLNSERMETHISGLYLYSTHRGLNALATHRSERGLYTHRILEGLNSERMETHISGLYLYSTHRGLNALATHRSERGLYTHRILEGLNSERMETHISGLYLYSTHRGLNALATHRSERGLYTHRILEGLNSERMETHISGLYLYSILEGLNALATHRSERGLYTHRILEGLNSER

(19) United States
(12) Patent Application Publication
(10) Pub. No.: US 2018/0223260 A1
(43) Pub. Date: Aug. 9, 2018

(54) FUNCTIONALIZED NANOPARTICLES FOR THE INTRACELLULAR DELIVERY OF BIOLOGICALLY ACTIVE MOLECULES AND METHODS FOR THEIR MANUFACTURE AND USE

(71) Applicant: STEMGENICS, INC., Kenmore, WA (US)

(72) Inventors: Andranik Andrew Aprikyan, Seattle, WA (US); Kilian Dill, Sultan, WA (US)

(73) Assignee: STEMGENICS, INC., Kenmore, WA (US)

(21) Appl. No.: 15/613,197

(22) Filed: Jun. 3, 2017

Related U.S. Application Data
(63) Continuation-in-part of application No. 14/353,280, filed on Apr. 21, 2014, now Pat. No. 9,675,708.
(60) Provisional application No. 61/550,213, filed on Oct. 21, 2011.
(60) Provisional application No. 62/345,360, filed on Jun. 3, 2016.
(60) Provisional application No. 62/406,542, filed on Oct. 11, 2016.
(60) Provisional application No. 62/406,838, filed on Oct. 11, 2016.

(51) Int. Cl. C12N 5/074; C12N 5/00; C12N 5/073

(52) U.S. Cl. CPC C12N 5/0696; C12N 5/0018; B82Y 5/00; C12N 2500/20; C12N 5/0603

(57) ABSTRACT

Provided are functionalized nanoparticles for penetrating through a mammalian cell membrane and delivering intracellularly one or more biologically active molecules comprising a nanoparticle core, one or more cell membrane-penetrating molecule(s), and one or more biologically active molecule(s) for introducing or affecting one or more cellular function(s). functionalized nanoparticles. Also provided are methods for making functionalized nanoparticles and methods for using functionalized nanoparticles, including methods for treating diseases and disorders, inducing the reprogramming of cells, and for gene editing.

### CROSS-REFERENCE TO RELATED APPLICATIONS

This application is a continuation-in-part of U.S. patent application Ser. No. 14/353,280, which was filed on Apr. 21, 2014 as a national phase application under 35 U.S.C. § 131 from PCT Patent Application No. PCT/US2012/061391, which was filed on Oct. 22, 2012, published on Apr. 25, 2013 as PCT Patent Publication No. WO 2013/059831, and claims the benefit of U.S. Provisional Patent Application No. 61/550,213, which was filed on Oct. 21, 2011.

This application also claims the benefit of U.S. Provisional Patent Application No. 62/345,360, filed Jun. 3, 2016, U.S. Provisional Patent Application No. 62/406,542, filed Oct. 11, 2016, and U.S. Provisional Patent Application No. 62/406,838, filed Oct. 11, 2016.

PCT Patent Application No. PCT/US2012/061391, U.S. patent application Ser. No. 14/353,280, and U.S. Provisional Patent Application Nos. 61/550,213, 62/345,360, 62/406,542, and 62/406,838 are incorporated herein by reference in their entirety.

### GOVERNMENT SPONSORED RESEARCH OR DEVELOPMENT

Certain aspects of this disclosure were made with Government support under Small Business Innovation Research (SBIR) Phase I IIP-1214943 awarded by the National Science Foundation. The Government has certain rights in one or more of the disclosures disclosed herein.

### BACKGROUND OF THE DISCLOSURE

### Technical Field

The present disclosure relates, generally, to nanotechnology and to the intracellular delivery of therapeutic agents. More specifically, the present disclosure provides functionalized nanoparticles comprising bioactive molecules (1) for intracellular delivery and (2) for regulating, modulating, and/or normalizing cellular functions including, for example, cell growth/proliferation, cell differentiation, and/or cell survival. Provided herein are methods for making functionalized nanoparticles comprising bioactive molecules and methods for using functionalized nanoparticles for the ex vivo or in vivo delivery of bioactive molecules to target cells, including: methods for the treatment of diseases and disorders; methods for generating stem cells (e.g., nanoparticle induced pluripotent stem cells (niPSC)), from somatic cells; methods for generating mature cell types by inducing the differentiation of stem cells (e.g., niPSC); methods for generating various mature cell types directly from other mature somatic cell types (i.e., direct reprogramming), and methods for gene editing. Also provided are methods for using cells that are generated with functionalized nanoparticles, including drug screening methods for identifying therapeutic drug candidates by contacting a target cell with member drugs from a drug library and selecting those drug candidates that confer a desired property in the target cell.

### Description of the Related Art

Nanoparticles made with paramagnetic, superparamagnetic, polymeric, and gold nanoparticle cores are known in the art and are have been used in a variety of applications, such as bioseparations, biophysical measurements, bioanalytical assays, therapeutics, and magnetic resonance imaging (TIM). For a general review of nanoparticle-based therapeutics that have received FDA approval or are in clinical trial, see Pillai, SOJ Pharmacy & Pharmaceutical Sciences 1(2): 13 (2014).

Applications for paramagnetic and superparamagnetic nanoparticles in the separation and purification of cells, viruses, and biological macromolecules are known in the art and are discussed, e.g., in Kemshead et al., European Journal of Cancer & Clinical Oncology 18(10):1043 (1982); Dirami et al., Journal Endocrinology 130(3):357-365 (1991); Ahmed et al., Biochemical Journal 286:377-382 (1992); Ito et al., Proc. Natl. Acad. Sci. U.S.A. 89(2):495-498 (1992); McConnell et al., Biotechniques 26(2):208 (1999); and U.S. Pat. Nos. 4,695,392 and 4,230,685, each of which is incorporated herein by reference in its entirety.

Paramagnetic and superparamagnetic nanoparticles may also be used for making biophysical measurements. For example, the capacity of paramagnetic and superparamagnetic nanoparticles to generate a force under a magnetic field has been exploited to distinguish between specific and nonspecific binding interactions and to characterize specific binding interactions as reported by, e.g., Shang et al., Journal of Magnetism and Magnetic Materials 293:382-388 (2005) and Strick et al., Science 271(5257):1835-1837 (1996).

Paramagnetic and superparamagnetic nanoparticles may also be used as bioanalytical tools. For example, U.S. Pat. No. 5,236,824 discloses an in situ laser magnetic immunoassay method that permits the quantification of a target immunological molecule in an analyte solution containing both bound and free species; U.S. Pat. No. 6,180,418 describes a force discrimination assay; and U.S. Pat. No. 6,294,342 describes qualitative and quantitative assay methods for measuring the association of specific binding pairs for detection of a desired analyte, which methods are based upon the response of magnetic particles to a magnetic field.

Paramagnetic and superparamagnetic nanoparticles have also been utilized in medical research, particularly in drug delivery and imaging. For example, paramagnetic and superparamagnetic nanoparticles have been used in conjunction with a magnetic and/or AC field (1) to direct therapeutic agents to specific target cells as described in Yellen et al., Journal of Magnetism and Magnetic Materials 293(1):647-654 (2005) and Saravanan et al., International Journal of Pharmaceutics 283(1-2):71-82 (2004); (2) to treat hyperthermia and to kill targeted cells, such as cancer cells, as described in Uskokovic et al., Materials Letters 60(21-22):2620-2622 (2006) and Jordan et al., Journal of Neuro-Oncology 78(1):7-14 (2006); and (3) as contrast agents to enhance the performance of MRI imaging as described in Dousset et al., American Journal of Neuroradiology 27 (5):1000-1005 (2006); McDonald et al., Academic Radiology 13(4):421-427 (2006); and Kleinschnitz et al., Journal of Cerebral Blood How and Metabolism 25(11):1548-1555 (2008).

Properties that are present only on the nanoscale level including, for example, switchable magnetic properties, make superparamagnetic iron oxide nanoparticles (also referred to as “SPIONs”) unique and are advantageously used in biomedical applications such as medical imaging and cell tracking. After intravenous administration, SPIONs are cleared from the blood via phagocytosis by the reticuloendothelial system (RES) and subsequently taken up by the liver, spleen, bone marrow, and lymph nodes. Upon intracellular uptake, SPIONs are metabolized in cellular lysosomes into a soluble, non-superparamagnetic form of iron that becomes part of the normal iron pool and incorporates into ferritin and hemoglobin in vivo.

Superparamagnetic nanoparticles for biomedical applications are reviewed in Neuberger et al., J. Magnetism and Magnetic Materials 293(1):483-496 (2005) and Hofmann-Amtenbrink et al., “Nanostructured Materials for Biomedical Applications,” Ch. 5 (Ed. Tan, Transworld Research Network, Kerala, India, 2009). Intracellular uptake of anionic superparamagnetic nanoparticles is reviewed in Wilhelm et al., Biomaterials 24(6):1001-1011 (2003).

Gold nanoparticles have been described for the delivery of drugs such as Paclitaxel. The delivery of hydrophobic molecules may be enhanced by encapsulation with a coating as discussed in further detain herein.

Gold nanoparticles are particularly effective in evading the reticuloendothelial system (RES) and may be used to circumvent multidrug resistance (MDR) mechanisms by, for example, enhancing drug uptake, activating cell efflux transporters to reduce intracellular drug concentration, altering cell cycle checkpoints to modify cellular pathways, increasing drug metabolism, inducing the expression of emergency response genes to impair apoptotic pathways, and altering DNA repair mechanisms.

Gold nanoparticle cores can be used as contrast agents for enhanced imaging and, because gold nanoparticles accumulate in tumors due to the leakiness of tumor vasculature, the functionalized gold nanoparticles disclosed herein may be used for cancer detection such as, for example, in a time-resolved optical tomography system using short-pulse lasers.

Intravenously-administered spherical gold nanoparticles broaden the temporal profile of reflected optical signals and enhance the contrast between tumors and surrounding normal tissue. Cancer cells reduce adhesion to neighboring cells and migrate into the vasculature-rich stroma. Once at the vasculature, cells can freely enter the bloodstream. Once the tumor is directly connected to the main blood circulation system, multifunctional nanocarriers can interact directly with cancer cells and effectively target tumors.

Therefore, gold nanoparticles have the potential to join numerous therapeutic functions into a single platform by targeting specific tumor cells, tissues and organs. Gene therapy is receiving increasing attention and, in particular, small-interference RNA (siRNA) shows importance in molecular approaches in the knockdown of specific gene expression in cancerous cells. The major obstacle to clinical application is the uncertainty about how to deliver therapeutic siRNAs with maximal therapeutic impact.

Gold nanoparticles have shown potential as intracellular delivery vehicles for siRNA oligonucleotides with maximal therapeutic impact. Conde et al. reported the use of siRNA/RGD gold nanoparticles capable of targeting tumor cells in two lung cancer xenograft mouse models, resulting in significant c-Myc oncogene downregulation followed by tumor growth inhibition and prolonged survival of the animals. This delivery system achieves translocation of siRNA duplexes directly into the tumour cell cytoplasm and accomplishes successful silencing of oncogene expression. RGD/siRNA-AuNPs can preferentially target and be taken up by tumor cells via integrin αvβ3-receptor-mediated endocytosis to selectively deliver c-Myc siRNA with no cytotoxicity, suppressing tumor growth and angiogenesis.

The ability of cells to normally proliferate, migrate, and differentiate into various cell types is critical in embryogenesis and in the function of mature cells. The functional ability of stem cells and/or more differentiated specialized cell types is altered in various pathological conditions. In most cases, mutant gene product that are implicated in pathogenesis and development of inherited or acquired human diseases, affect distinct intracellular events, which lead to abnormal cellular functions and the specific disease phenotype.

For example, abnormal cellular functions such as impaired survival and/or differentiation of bone marrow stem/progenitor cells into neutrophils are associated with patients having cyclic or severe congenital neutropenia (reduced levels of blood neutrophils) who may suffer from severe life-threatening infections and may develop acute myelogenous leukemia or other malignancies. Aprikyan et al., Blood 97:147 (2001) and Carlsson et al., Blood 103:3355 (2004).

Inherited or acquired disorders such as severe congenital neutropenia or Barth syndrome are associated with various gene mutations and are due to deficient production and function of a patient's blood and/or cardiac cells leading to subsequent neutropenia, cardiomyopathy, and/or heart failure. Makaryan et al., Eur. J. Haematol. 88:195-209 (2012). Severe congenital neutropenia disease phenotype can, for example, be caused by one or more substitution, deletion, insertion, and/or truncation mutations in the neutrophil elastase gene, HAXI gene, or Wiskott Aldrich Syndrome Protein genes. Dale et al., Blood 96:2317-2322 (2000); Devriendt et al., Nat. Genet. 27:313-7 (2001); and Klein et al., Nat. Genet. 39:86-92 (2007).

Other inherited diseases like Barth syndrome, a multi-system stem cell disorder induced by loss-of-function mutations in the mitochondrial TAZ gene, are associated with neutropenia. Neutropenia may cause recurring severe and sometimes life-threatening infections and/or cardiomyopathy that may lead to heart failure requiring heart transplantation.

Treatment of patients with granulocyte colony-stimulating factor (G-CSF) induces conformational changes in the cell-surface expressed G-CSF receptor, which triggers a chain of intracellular events that ultimately restore neutrophil production to near-normal levels and improves patient quality of life. Welte and Dale, Ann. Hematol. 72:158 (1996). Nonetheless, patients treated with G-CSF may develop leukemia. Aprikyan et al., Exp. Hematol. 31:372 (2003); Rosenberg et al., Br. J. Haematol. 140:210 (2008); and Newburger et al., Blood Cancer 55:314 (2010); and Aprikyan and Khuchua, Br. J. Haematol. 161:330 (2013). It is for these and other reasons that novel therapeutic regimen are being explored.

The intracellular events of pathological stem and other cells can be more effectively affected and regulated upon intracellular delivery of various biologically active molecules. These bioactive molecules may normalize the targeted cellular function or eliminate unwanted cells when needed. The cell membrane, however, serves as an active barrier preserving the cascade of intracellular events from being affected by exogenous stimuli.

For this reason, the ability to penetrate the cell membrane is often critical to the development of efficacious small-molecule-based therapeutics. For example, the impaired survival and differentiation of human bone marrow progenitor cells into neutrophils that is observed in patients with cyclic or severe congenital neutropenia may be normalized by a cell membrane-penetrant small molecule inhibitor of neutrophil elastase, which interferes with aberrant intracellular events thereby restoring a normal phenotype.

Such small molecules that are specific to mutant protein that cause disease are rarely available. Thus, there remains a need for therapeutic compounds and compositions that can efficiently penetrate the cell membrane to achieve intracellular delivery of biologically active molecules that are capable of modulating a desired cellular function.

Despite these advances in nanoparticle technologies, there remains a need in the art for agents that permit the target cell-specific access to intracellular compartments and the regulation, modulation, normalization, and/or restoration of one or more desired cellular functions. The present disclosure fulfills these needs and provides further related advantages.

### SUMMARY OF THE DISCLOSURE

The present disclosure addresses unmet needs in the art by providing functionalized nanoparticles, including functionalized paramagnetic and superparamagnetic nanoparticles, functionalized non-magnetic nanoparticles, such as functionalized gold nanoparticles, and functionalized polymeric nanoparticles, for the intracellular delivery of biologically active molecules to introduce or affect one or more cellular functions.

Within certain embodiments, the present disclosure provides functionalized nanoparticles, and methods for making functionalized nanoparticles, for: (i) the treatment of diseases and disorders, including cancers, neurological diseases, and cardiac disorders; (ii) inducing the reprogramming of somatic cells, including fibroblasts, into stem cells, such as nanoparticle induced pluripotent stem cells (niPSCs); (iii) inducing the reprogramming, including direct reprogramming, of cells, such as somatic cells and stem cells, including niPSCs, into differentiated cell types, including hematopoietic cells, neuronal cells, hepatic cells, and cardiac cells; and (iv) gene editing and repair of genetic mutations.

The present disclosure also provides methods for the use of the functionalized nanoparticles disclosed herein for: (i) the treatment of human diseases and disorders; (ii) reprogramming differentiated cells into undifferentiated cells, including stem cells, such as induced pluripotent stem cells (iPSC); (iii) producing differentiated cells by inducing the differentiation of undifferentiated cells, including stem cells, such as nanoparticle induced pluripotent stem cells (niPSC); and (iv) gene editing and repair of genetic mutations.

The functionalized nanoparticles disclosed herein include: (i) one or more targeting molecules, which include cell membrane-penetrating molecules for penetrating through a mammalian cell membrane (e.g., a plasma membrane, a nuclear membrane, a mitochondrion membrane, a lysosomal membrane, an endosomal membrane, and/or other organelle membrane) and, thereby, facilitating the intracellular delivery of the functionalized nanoparticle and (ii) one or more biologically active molecules for affecting one or more cellular functions such as, for example, normalizing, restoring, regulating, and/or modulating (i.e., stimulating or inhibiting) one or more cellular functions such as, for example, cell maintenance, survival, growth/proliferation, differentiation, and/or death.

Within certain embodiments, the present disclosure provides functionalized nanoparticles having (a) a nanoparticle core; (b) one or more targeting molecules, which include cell membrane-penetrating molecule(s); and (c) one or more biologically active molecule(s) for regulating, modulating, normalizing, and/or restoring one or more cellular function(s), wherein each of said one or more cell membrane-penetrating molecule(s) and each of said biologically active molecule(s) is attached directly to the nanoparticle core.

Within other embodiments, the present disclosure provides functionalized nanoparticles having (a) a nanoparticle core; (b) one or more functional group(s) that are associated with and/or attached directly to the nanoparticle; (c) one or more targeting molecules, which include cell membrane-penetrating molecule(s); and (d) one or more biologically active molecule(s) for regulating, modulating, normalizing, and/or restoring one or more cellular functions, wherein each cell membrane-penetrating molecule and each biologically active molecule is attached to the nanoparticle via the one or more functional group(s).

Within yet other embodiments, the present disclosure provides functionalized nanoparticles having (a) a nanoparticle core; (b) one or more functional group(s) that are associated with and/or attached directly to the nanoparticle; (c) first and second crosslinking agent(s) each having first and second functional groups, wherein said first crosslinking agent has a first length and said second crosslinking agent has a second length; (d) one or more cell membrane-penetrating molecule(s); and (e) one or more biologically active molecule(s) for regulating, modulating, normalizing, and/or restoring one or more cellular functions, wherein each cell membrane-penetrating molecule and each biologically active molecule is attached to the nanoparticle via the one or more functional group(s).

Within further embodiments, the present disclosure provides functionalized nanoparticles having (a) a nanoparticle core; (b) a polymer coating or lipid bilayer that encapsulates the nanoparticle; (c) one or more functional group(s) that are associated with and/or attached directly to the nanoparticle and/or to the polymer coating or lipid bilayer; (d) one or more cell membrane-penetrating molecule(s); and (e) one or more biologically active molecule(s) for regulating, modulating, normalizing, and/or restoring one or more cellular functions, wherein each cell membrane-penetrating molecule and each biologically active molecule is attached to a functional group on the nanoparticle and/or to a functional group on the polymer coating or lipid bilayer.

Within still further embodiments, the present disclosure provides functionalized nanoparticles having (a) a nanoparticle core; (b) a polymer coating or lipid bilayer that encapsulates the nanoparticle; (c) one or more functional group(s) that are associated with and/or attached directly to the nanoparticle and/or to the polymer coating or lipid bilayer; (d) first and second crosslinking agent(s) each having first and second functional groups, wherein said first crosslinking agent has a first length and said second crosslinking agent has a second length; (e) one or more cell membrane-penetrating molecule(s); and (f) one or more biologically active molecule(s) for regulating, modulating, normalizing, and/or restoring one or more cellular functions, wherein each crosslinking agent is attached to the nanoparticle and/or to the polymer coating or lipid bilayer via a first functional group and wherein each cell membrane-penetrating molecule and each biologically active molecule is attached to a second functional group on the crosslinking agent.

Suitable nanoparticle cores that may be employed in each of these embodiments include metallic, ceramic, and synthetic nanoparticle cores having hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Metallic nanoparticle cores include magnetic nanoparticles, including iron-containing nanoparticle cores, such as paramagnetic nanoparticle cores and superparamagnetic nanoparticle cores; gold nanoparticle cores; as well as nanoparticle cores made with one or more additional metals including any one of, or combination of two or more of, aluminum, barium, beryllium, chromium, cobalt, copper, iron, manganese, magnesium, strontium, zinc, rare earth metal, or trivalent metal ion. Other metal species, such as silicon oxide, silver, titanium, and ITO can also be used in the presently disclosed nanoparticle cores.

Suitable polymer coatings or lipid bilayers that may be used in the functionalized nanoparticles disclosed herein include, for example, those polymer coatings or lipid bilayers that (1) reduce nanoparticle cytotoxicity, (2) increase nanoparticle hydrophilicity or hydrophobicity, and/or (3) to provide a surface that can be modified with one or more functional groups for attachment to one or more crosslinking agents, biologically active molecules, and/or cell membrane-penetrating molecules.

Suitable functional groups that may be used in the functionalized nanoparticles disclosed herein include, for example, amino groups (—NH₂), sulfhydryl groups (—SH), carboxyl groups (—COOH), guanidyl groups (—NH₂—C(NH)—NH₂), hydroxyl groups (—OH), azido groups (—N₃), and/or carbohydrates. Such functional groups can attach directly to a biologically active molecule, a cell membrane-penetrating molecule, and/or a crosslinking agent through, for example, an amino, sulfhydryl, or phosphate group. Alternatively, a functional group can be provided as a functionalized polymer that is formed, for example, on a synthetic nanoparticle shell.

Functional groups may also include one or more stabilizing groups, such as stabilizing groups selected from the group consisting of phosphate; diphosphate, carboxylase, polyphosphate, thiophosphate, phosphonate, thiophosphonate, sulphate, sulphonate, mercapto, silanetriol, trialkoxysilane-containing polyalkylene glycols, polyethylene glycols, carbohydrate or phosphate-containing nucleotides, oligomers thereof or polymers thereof.

Suitable crosslinking agents that may be used in the functionalized nanoparticles disclosed herein include long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pyridyldithio)-proprionate (SPDP); long-chain N-Succinimidyl-3-(pyridyldithio)-proprionate (LC-SPDP); sulfo-N-Succinimidyl-3-(pyridyldithio)-proprionate (sulfo-SPDP); long-chain sulfo-N-Succinimidyl-3-(pyridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pyridyldithio)-proprionate polyethylene glycolₘ (SPDP(PEG)ₘ); and sulfo-N-Succinimidyl-3-(pyridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

Suitable biologically active molecules that may be used in the functionalized nanoparticles disclosed herein include one or more biologically active molecule(s) that introduce one or more new function(s) to a cell or regulate, modulate, and/or normalize one or more cellular function(s) such as cell maintenance/survival, cell growth/proliferation, cell differentiation, and/or cell death. Within certain aspects, biologically active molecules include, but are not limited to antibodies, full-length proteins, polypeptides, and/or peptides; nucleic acids, such as cDNAs, RNAs, oligonucleotides, primers, and probes; and/or small molecules that can regulate, modulate, normalize, provide, and/or restore one or more cellular function(s), such as cell maintenance, survival, growth/proliferation, differentiation, and/or death.

Suitable targeting molecules that may be used in the functionalized nanoparticles disclosed herein include, for example, full-length proteins, polypeptides, and/or peptides; nucleic acids, such as cDNAs, RNAs, oligonucleotides, primers, and/or probes; and/or small molecules to facilitate the specific delivery of a functionalized nanoparticle to a target cell. Targeting molecules include cell membrane-penetrating molecules, which facilitate the (i) the cellular uptake of a functionalized nanoparticle through a mammalian cell plasma membrane and, optionally, (ii) the subcellular localization of a functionalized nanoparticle into, for example, a mammalian cell nucleus, mitochondria, endosome, lysosome, or other organelle via a mammalian cell nuclear membrane, mitochondrial membrane, lysosomal membrane, endosomal membrane, and/or other organelle membrane.

Suitable cell membrane-penetrating molecules that may be used in the functionalized nanoparticles disclosed herein include full-length proteins, polypeptides, peptides, nucleic acids, and small molecules. Exemplary peptides include those deriving from HIV Tat as well as peptides having from five to nine or more basic amino acids, such as lysine and arginine, and include peptides having from five to nine or more contiguous basic amino acids, such as lysine and arginine.

The present disclosure provides functionalized nanoparticles that may be advantageously employed in (1) methods for the treatment of diseases and disorders, in particular human diseases and disorders; (2) methods for inducing the reprogramming of mammalian cells, including somatic cells and stem cells; (3) methods for promoting the repair of target nucleic acids; and (4) methods for gene editing.

Thus, within certain embodiments, provided herein are methods for using the presently disclosed functionalized nanoparticles, which methods include contacting a cell with a functionalized nanoparticle that comprises (1) a biologically active molecule for effectuating (i.e., regulating, modulating, normalizing, and/or restoring) one or more functions of the cell such as, for example, maintenance, survival, growth/proliferation, differentiation, and/or death and (2) a targeting molecule, such as a cell membrane-penetrating molecule for binding to and penetrating a membrane of the cell, including a plasma membrane, a nuclear membrane, a mitochondrion membrane, an endosomal membrane, a lysosomal membrane, and/or other membrane, thereby facilitating the delivery of the functionalized nanoparticle to the cell and effectuating the one or more cellular functions by the biologically active molecule.

Within other embodiments, provided herein are methods for using the presently disclosed functionalized nanoparticles, which methods include administering to a patient having a disease or disorder a functionalized nanoparticle that comprises (1) a biologically active molecule for effectuating (i.e., regulating, modulating, normalizing, and/or restoring) one or more functions of a cell within the patient such as, for example, maintenance, survival, growth/proliferation, differentiation, and/or death and (2) a targeting molecule, such as a cell membrane-penetrating molecule for binding to and penetrating a membrane of a cell of the patient having a disease or disorder, including a plasma membrane, a nuclear membrane, a mitochondrion membrane, an endosomal membrane, a lysosomal membrane, and/or other membrane, thereby facilitating the delivery of the functionalized nanoparticle to the cell and effectuating the one or more cellular functions by the biologically active molecule thereby alleviating one or more aspects of the disease or disorder.

Within further embodiments, the present disclosure provides functionalized nanoparticles for promoting the differentiation of cells into induced cardiomyocyte-like cells (iCMs).

Within certain aspects of these embodiments, functionalized nanoparticles include (a) a nanoparticle core having first and second functional groups that are associated with and/or attached directly to the nanoparticle core; (b) one or more cell targeting molecule(s), including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a cardiomyocyte inducing agent or a nucleic acid encoding a cardiomyocyte inducing agent and wherein one or more of the cell targeting molecule(s) is attached directly to the nanoparticle core via a first functional group on the nanoparticle core and one or more of the biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a cardiomyocyte inducing agent or a nucleic acid encoding a cardiomyocyte inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a cardiomyocyte inducing agent or a nucleic acid encoding a cardiomyocyte inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a cardiomyocyte inducing agent or a nucleic acid encoding a cardiomyocyte inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable cardiomyocyte inducing agents that may be employed in functionalized nanoparticles according to these embodiments include, for example, Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and c-Myc, or a functional domain or structural variant thereof. More specifically, suitable cardiomyocyte inducing agents include: (1) one or more of Gata4, MEF2C, TBX5, MESP1, Hand2, MYOCD, miR-1, and miR-133, or a functional domain or structural variant thereof; one or more of Gata4, MEF2C, TBX5, MESP1, Hand2, MYOCD, miR-1, and miR-133, or a functional domain or structural variant thereof; (3) one or more of Gata4, MEF2C, TBX5, MESP1 and MYOCD, or a functional domain or structural variant thereof; or (4) one or more of Gata4, Hand2, TBX5, MYOCD, miR-1, and miR-133, or a functional domain or structural variant thereof.

The present disclosure further provides methods for manufacturing a functionalized nanoparticle for promoting the differentiation of a cell into an induced cardiomyocyte-like cell (iCM), which methods include attaching a cardiomyocyte inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and c-Myc, or a functional domain or structural variant thereof.

The present disclosure also provides methods for promoting the differentiation of a cell into an induced cardiomyocyte-like cell (iCM), which methods include contacting the cell with a functionalized nanoparticle comprising a nanoparticle core, including a metal nanoparticle core, such as an iron or gold containing nanoparticle core, a synthetic nanoparticle core, or a ceramic nanoparticle core, to which a cardiomyocyte inducing agent and a cell targeting molecule is attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4 and c-Myc, or a functional domain or structural variant thereof.

Within some embodiments, the present disclosure provides functionalized nanoparticles for promoting the differentiation of cells into an induced pluripotent stem cells (iPSCs).

Within certain aspects of these embodiments, functionalized nanoparticles include (a) a nanoparticle core having first and second functional groups that are associated with and/or attached directly thereto; (b) one or more cell targeting molecule(s), including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a stem cell inducing agent or a nucleic acid encoding a stem cell inducing agent and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a stem cell inducing agent or a nucleic acid encoding a stem cell inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a stem cell inducing agent or a nucleic acid encoding a stem cell inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a stem cell inducing agent or a nucleic acid encoding a stem cell inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable stem cell inducing agents that may be employed in functionalized nanoparticles according to these embodiments include, for example, Lin28, Nanog, Mir-302bcad/367, Mir-302, Mir-200c, Mir-369, Oct4, Sox2, Klf4, and c-Myc, or a functional domain or structural variant thereof. In some applications, functionalized nanoparticles include two, three, four, five, or more stem cell inducing factors each of which is independently selected from the group consisting of Lin28, Nanog, Mir-302bcad/367, Mir-302, Mir-200c, Mir-369, Oct4, Sox2, Klf4, and c-Myc, or a functional domain or structural variant thereof.

The present disclosure further provides methods for manufacturing functionalized nanoparticles for promoting the differentiation of cells into induced pluripotent stem cells (iPSCs), which methods include attaching a stem cell inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Lin28, Nanog, Mir-302bcad/367, Mir-302, Mir-200c, Mir-369, Oct4, Sox2, Klf4, and c-Myc, or a functional domain or structural variant thereof.

The present disclosure also provides methods for promoting the differentiation of a cell into an induced pluripotent stem cell (iPSC), which methods include contacting the cell with a functionalized nanoparticle comprising a nanoparticle core, including a metal nanoparticle core, such as an iron or gold containing nanoparticle core, a synthetic nanoparticle core, or a ceramic nanoparticle core, to which a stem cell inducing agent and a cell targeting molecule is attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Lin28, Nanog, Mir-302bcad/367, Mir-302, Mir-200c, Mir-369, Oct4, Sox2, Klf4, and c-Myc, or a functional domain or structural variant thereof.

The present disclosure also provides methods for the treatment of a neurodegenerative disease or disorder in a patient, which methods include administering to the patient a functionalized nanoparticle comprising a nanoparticle core to which a stem cell inducing agent and a cell targeting molecule are attached. Neurodegenerative diseases or disorders that may be treated according to these methods include, for example, leukencephalopathy, leukodystrophy, Adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, pigmented orthochromatic leukodystrophy (POLD), Schizophrenia, Bipolar Disorder, Autism, idiopathic leukencephalopathy, Niemann Pick Disease, Nasu-Hakola Disease, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy) or is a condition characterized by apraxia, achalasia, and epilepsy.

The present disclosure also provides methods for the treatment of a neurodegenerative disease or disorder in a patient, which methods include administering to the patient an iPSC produced by methods and using functionalized nanoparticles as disclosed herein. Neurodegenerative diseases or disorders that may be treated according to these methods include, for example, leukoencephalopathy, leukodystrophy, Adult onset leukoencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, pigmented orthochromatic leukodystrophy (POLD), Schizophrenia, Bipolar Disorder, Autism, idiopathic leukencephalopathy, Niemann Pick Disease, Nasu-Hakola Disease, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy) or is a condition characterized by apraxia, achalasia, and epilepsy.

Within some embodiments, the present disclosure provides functionalized nanoparticles for promoting the differentiation of cells into induced neuronal cells (iNCs).

Within certain aspects of these embodiments, functionalized nanoparticles include (a) a nanoparticle core having first and second functional groups that are associated with and/or attached directly to the nanoparticle core; (b) one or more cell targeting molecules, including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a neuronal cell inducing agent or a nucleic acid encoding a neuronal cell inducing agent and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a neuronal cell inducing agent or a nucleic acid encoding a neuronal cell inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a neuronal cell inducing agent or a nucleic acid encoding a neuronal cell inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a neuronal cell inducing agent or a nucleic acid encoding a neuronal cell inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable neuronal cell inducing agents that may be employed in functionalized nanoparticles according to these embodiments include, for example, Brn2, Asc11, Myt11, Zic1, Mir-9, Mir-124, NeuroD1, Lmx1a, FoxA2, Oct4, Sox2, Klf4, c-Myc, or a functional domain or structural variant thereof. In some applications, functionalized nanoparticles include two, three, four, five, or more stem cell inducing factors each of which is independently selected from the group consisting of Brn2, Asc11, Myt11, Zic1, Mir-9, Mir-124, NeuroD1, Lmx1a, FoxA2, Oct4, Sox2, Klf4, c-Myc, or a functional domain or structural variant thereof.

The present disclosure further provides methods for manufacturing functionalized nanoparticles for promoting the differentiation of cells into induced neuronal cells (iNCs), which methods include attaching a neuronal cell inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable neuronal cell inducing agents include Brn2, Asc11, Myt11, Zic1, Mir-9, Mir-124, NeuroD1, Lmx1a, FoxA2, Oct4, Sox2, Klf4, c-Myc, or a functional domain or structural variant thereof.

The present disclosure also provides methods for promoting the differentiation of a cell into an induced neuronal cell (INC), which methods include contacting a cell with a functionalized nanoparticle comprising a nanoparticle core, including a metal nanoparticle core, such as an iron or gold containing nanoparticle core, a synthetic nanoparticle core, or a ceramic nanoparticle core, to which a neuronal cell inducing agent and a cell targeting molecule are attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable neuronal cell inducing agents include Brn2, Asc11, Myth 1, Zic1, Mir-9, Mir-124, NeuroD1, Lmx1a, FoxA2, Oct4, Sox2, Klf4, c-Myc, or a functional domain or structural variant thereof.

The present disclosure also provides methods for the treatment of a neurodegenerative disease or disorder in a patient, which methods include administering to the patient a functionalized nanoparticle comprising a nanoparticle core to which a neuronal cell inducing agent and a cell targeting molecule are attached. Neurodegenerative diseases or disorders that may be treated according to these methods include, for example, leukencephalopathy, leukodystrophy, Adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, pigmented orthochromatic leukodystrophy (POLD), Schizophrenia, Bipolar Disorder, Autism, idiopathic leukencephalopathy, Niemann Pick Disease, Nasu-Hakola Disease, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy) or is a condition characterized by apraxia, achalasia, and epilepsy.

The present disclosure also provides methods for the treatment of a neurodegenerative disease or disorder in a patient, which methods include administering to the patient a functionalized nanoparticle comprising a nanoparticle core to which is attached (a) one or more biologically active molecule(s) such as (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including one or more a cell membrane-penetrating molecules, such as an HIV Tat derived peptide or other peptide having, for example, from five to nine basic amino acids, including arginine and/or lysine.

Neurodegenerative diseases or disorders that may be treated according to these methods include, for example, leukencephalopathy, leukodystrophy, Adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, pigmented orthochromatic leukodystrophy (POLD), Schizophrenia, Bipolar Disorder, Autism, idiopathic leukencephalopathy, Niemann Pick Disease, Nasu-Hakola Disease, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy) or is a condition characterized by apraxia, achalasia, and epilepsy.

Within certain aspects of these embodiments, the neurodegenerative disease or disorder is Nasu-Hakola disease and the target nucleic acid is the CSF-1R gene. Within other aspects, the neurodegenerative disease or disorder is Nasu-Hakola disease and the target nucleic acid is the TREM2 gene. Within further aspects, the neurodegenerative disease or disorder is metachromatic leukodystrophy (MLD) disease and the target nucleic acid is the Arylsulfatase A gene.

Within some embodiments, the present disclosure provides functionalized nanoparticles for promoting the repair of genetic mutations in target nucleic acids, which functionalized nanoparticles include (a) a nanoparticle core; (b) one or more functional group(s) that are associated with and/or attached directly to the nanoparticle; (c) one or more cell targeting molecules, including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other embodiments, the present disclosure provides functionalized nanoparticles for promoting the repair of genetic mutations in target nucleic acids, which functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further embodiments, the present disclosure provides functionalized nanoparticles for promoting the repair of genetic mutations in target nucleic acids, which functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further embodiments, the present disclosure provides functionalized nanoparticles for promoting the repair of genetic mutations in target nucleic acids, which functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

The present disclosure further provides methods for manufacturing functionalized nanoparticles for promoting the repair of a genetic mutation in a target nucleic acid, which methods include attaching to a nanoparticle core, including a metal nanoparticle core, such as an iron or gold containing nanoparticle core, a synthetic nanoparticle core, or a ceramic nanoparticle core: (a) a biologically active molecule selected from (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including a cell membrane-penetrating molecule, such as an HIV Tat derived peptide or other peptide having, for example, from five to nine basic amino acids, including arginine and/or lysine. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

The present disclosure further provides methods for promoting the repair of a genetic mutation in a target nucleic acid, such as (1) a CSF-1R gene, including a CSF-1R gene having a genetic mutation in a region encoding a tyrosine kinase domain, (2) a TREM2 gene, and (3) an Arylsulfatase A (ARSA) gene. These methods include contacting the cell with a functionalized nanoparticle comprising a nanoparticle core to which is attached (a) a biologically active molecule that is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including a cell membrane-penetrating molecule, such as an HIV Tat derived peptide or other peptide having, for example, from five to nine basic amino acids, including arginine and/or lysine.

Within certain aspects, such functionalized nanoparticles may further include a donor nucleic acid molecule comprising a nucleotide sequence for insertion into the cleavage site of said target nucleic acid. Suitable nanoparticle cores include metal nanoparticle cores, such as an iron or gold containing nanoparticle cores, synthetic nanoparticle cores, and ceramic nanoparticle cores having hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

Within other aspects of these methods, the functionalized nanoparticle employs CRISPR technology and the nuclease comprises a functional domain of Cas9, nickase, Ago, or a homolog thereof. Within these or related aspects of these methods, both the guide nucleic acid and the nuclease or nucleic acid encoding the nuclease are attached to the nanoparticle core. Within other aspects of these methods, only one of the guide nucleic acid and the nuclease or nucleic acid encoding the nuclease is attached to the nanoparticle core.

Within further embodiments, the present disclosure provides methods for genome correction and modulation of cellular functions, which methods comprise contacting a cell or administering to a patient one or more functionalized nanoparticles as disclosed herein.

Within other embodiments, the present disclosure provides methods for the direct reprogramming of a somatic cell, such as a fibroblast or other differentiated somatic cell, into a functional cell having a selected (predetermined) lineage such as a cardiac cell, a hepatocyte, and a neural cell. Within other aspects of these embodiments, the present disclosure provides methods for the direct reprogramming of a somatic cell, such as a fibroblast or other differentiated somatic cell, into a stem cell, such as an induced pluripotent stem cell (iPSC) or other undifferentiated cell type.

Within certain aspects of these embodiments are provided methods for the direct reprogramming of a somatic cell, such as a fibroblast or other differentiated somatic cell, that is obtained from a human subject that is afflicted with a neurodegenerative disease or disorder or at risk for developing a neurodegenerative disease or disorder. As disclosed herein, somatic cells may, for example, be obtained from a human subject that is afflicted with a neurodegenerative disease or disorder that is selected from leukencephalopathy, leukodystrophy, adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g., stroke, head injury, cerebral palsy) or is a condition characterized by apraxia, achalasia, or epilepsy.

In related embodiments, the present disclosure provides methods for treating a neurodegenerative diseases and disorders in a subject that is afflicted with a neurodegenerative disease or disorder or at risk for developing a neurodegenerative disease or disorder. Such methods comprise administering specialized cell types that are generated from one or more cells that are sourced from the subject and that include, without limitation, induced pluripotent stem cells that are induced from somatic cells that are contacted with a functionalized nanoparticle as disclosed herein.

In certain aspects of these embodiments, methods employ non-integrating functionalized nanoparticles to reprogram somatic cells from a patient afflicted with or at risk for developing a neurodegenerative diseases or disorder, wherein the somatic cells are reprogrammed into pluripotent stem cells that are contacted with a functionalized nanoparticle as disclosed herein.

In other aspects of these embodiments, the induced pluripotent stem cells are made from somatic cells from an individual with a genetic leukodystrophy wherein the gene has been corrected prior to administration to the subject. Gene correction may employ a CRISPR/Cas9 gene editing system or other gene editing approaches available in the art such as, for example, CRISPR nanoparticle conjugates as described in U.S. Patent Application No. 62/406,542, incorporated herein by reference in its entirety, and/or as described elsewhere herein. The source somatic cells can be any nucleated cell from the patient, such as skin or blood cells, including monocytes.

The gene that is corrected may be the CSF-1R gene, particularly in the region of the gene coding for the tyrosine kinase function. Alternatively, the gene that is corrected may be TREM2 or ARSA. The gene that is corrected may be related to a patient's hematopoietic disorder, such as a hematopoietic disorder that may evolve into a leukemia. In such cases, the target gene for correction can include ELANE (a neutrophil elastase gene), HAX-1, WAS, or one or more other gene(s) that, when mutated, contribute to a hematopoietic disorder.

In further aspects of these embodiments, induced pluripotent stem cells may be derived from blood cells or skin cells from a patient with ALSP that has a mutation in the CSF-1R gene that is subsequently corrected by CRISPR and/or nanoparticle conjugates. Corrected induced pluripotent stem cells may be expanded ex vivo and differentiated into macrophages, microglia, or neural progenitors prior to administration to the patient in sufficient quantity to reduce or ameliorate disease symptoms, and/or to restore normal function in vivo. It will be appreciated that the order of pluripotency and gene editing steps can be altered to reach similar results. Thus, a patient's cells can alternatively be used first to correct a mutant gene with subsequent reprogramming into the appropriate state, such as nanoparticle-induced pluripotent stem or other more specialized cell type as needed.

Induced pluripotent stem cells may be differentiated into either macrophages, microglia or neural progenitors prior to genetic correction and used in the presently disclosed methods for treating or preventing a neurodegenerative disease in a subject by administering to the subject an effective amount of genetically corrected induced pluripotent stem cell-derived cells including, but not limited to, macrophages, microglia, or neural progenitor cells. Within certain aspects, these methods optionally include selecting a subject with a neurodegenerative disease of the central nervous system (for example, at an early stage of disease) or at risk for a neurodegenerative disease of the central nervous system.

Within certain methods, an effective amount of corrected induced pluripotent stem cells are administered to a patient to diminish or ameliorate a disease state and/or to restore normal function in neural cells, ameliorate symptoms of neural degeneration, and/or prevent or inhibit the onset of symptoms in a patient predisposed to a neuronal disease or disorder by way of genetic mutations. The method optionally includes the determination and/or confirmation that a subject has a mutation in the CSF-1R gene, the TREM2 gene, and/or the ARSA gene as compared to normal control subjects. Such a determination can be performed before the generation of gene-corrected induced pluripotent stem cells.

Within certain aspects of these methods, the patient may be diagnosed as having, suspected of having, or at risk of having ALSP or Multiple Sclerosis, Parkinson's Disease, periventricular leukomalacia, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD, Rett Disease, apraxia, achalasia, epilepsy, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g., stroke, head injury, cerebral palsy, and the like).

Neurological diseases and disorders that are treatable using the methods disclosed herein include, but are not limited to, Adult onset leukencephalopathy with spheroids and pigmented Glia (ALSP), Multiple Sclerosis, Parkinson's Disease, periventricular leukomalacia, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD), Rett Disease, or is a condition characterized by apraxia, achalasia, epilepsy, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelolinosis, spongiform leukodystrophy, and leukodystrophy (Alexander type); and acute brain injury (e.g., stroke, head injury, cerebral palsy).

Acquired or inherited hematopoietic diseases and disorders that are treatable using the methods disclosed herein include cyclic neutropenia, myelokathexis, severe congenital neutropenia, acute myeloid leukemia, and lymphoblastic leukemias that are due to mutations in corresponding genes.

It will be understood that various changes, alterations, and substitutions may be made to the various embodiments disclosed herein without departing from their essential spirit and scope.

### BRIEF DESCRIPTION OF THE DRAWINGS

Certain aspects of the present disclosure will become more evident in reference to the drawings, which are presented for illustration, not limitation.

FIGS. 1A and 1B depict a multi-step scheme for the functionalization of nanoparticles, which is based on the simultaneous attachment of peptide and protein molecules to a nanoparticle in accordance with an embodiment of the present disclosure.

FIG. 2A depicts a reaction of a nanoparticle containing amine groups with equimolar ratios of long chain LC1-SPDP and iodoacetic acid nanoparticle in accordance with an embodiment of the present disclosure.

FIG. 2B depicts a reduction of the disulfide bond of PDP to provide a free SH group nanoparticle in accordance with an embodiment of the present disclosure.

FIG. 2C depicts a reaction of long chain LC1-SMCC with the lysine groups of a protein nanoparticle in accordance with an embodiment of the present disclosure.

FIG. 2D depicts a reaction of a multifunctional nanoparticle with the protein that had been reacted with SMCC and contains a terminal reactive maleimide group nanoparticle in accordance with an embodiment of the present disclosure.

FIG. 2E depicts a reaction of an amino group of a peptide with LC2-SMCC. The reaction is then subsequently followed by a reaction with mercaptoethanol to convert the terminal maleimide to an alcohol nanoparticle in accordance with an embodiment of the present disclosure.

FIG. 2F depicts a reaction of a functional bead (and protein attached) with a modified peptide to the free carboxyl group on the nanoparticle in accordance with an embodiment of the present disclosure.

FIG. 3A depicts a reaction of a nanoparticle containing amine groups with LC1-SPDP nanoparticle in accordance with an embodiment of the present disclosure.

FIG. 3B depicts a reduction of the disulfide bond of PDP to provide a free SH group nanoparticle in accordance with an embodiment of the present disclosure.

FIG. 3C depicts a reaction of long chain LC2-SMCC with the lysine groups of a protein nanoparticle in accordance with an embodiment of the present disclosure.

FIG. 3D depicts a reaction of a multifunctional nanoparticle with the protein that had been reacted with SMCC and contains a terminal reactive maleimide group nanoparticle in accordance with an embodiment of the present disclosure.

### DETAILED DESCRIPTION OF THE DISCLOSURE

The present disclosure is based upon the development of functionalized nanoparticles, including functionalized paramagnetic, functionalized superparamagnetic, polymeric, and functionalized gold nanoparticles, which are configured for the intracellular delivery of biologically active molecules that affect or introduce one or more cellular functions and/or activities. This disclosure will be better understood in view of the following definitions, which are provided for clarification and are not intended to limit the scope of the subject matter that is disclosed herein.

### Definitions

Unless specifically defined otherwise herein, each term used in this disclosure has the same meaning as it would to those having skill in the relevant art. General guidance for certain aspects of the present disclosure may be found in Sambrook, et al. (eds.), Molecular Cloning: A Laboratory Manual, 3rd ed., Cold Spring Harbor Press, Plainsview, N.Y. (2001) and Ausubel, et al. (eds.), Current Protocols in Molecular Biology, John Wiley & Sons, New York (2010).

Words and phrases using the singular or plural number also include the plural and singular number, respectively. For example, terms such as “a” or “an” and phrases such as “at least one” and “one or more” include both the singular and the plural. Terms that are intended to be “open” (including, for example, the words “comprise,” “comprising,” “include,” “including,” “have,” and “having,” and the like) are to be construed in an inclusive sense as opposed to an exclusive or exhaustive sense. That is, the term “including” should be interpreted as “including but not limited to,” the term “includes” should be interpreted as “includes but is not limited to,” the term “having” should be interpreted as “having at least.”

The use of the term “or” in the claims is used to mean “and/or” unless explicitly indicated to refer to alternatives only or the alternatives are mutually exclusive, although the disclosure supports a definition that refers to only alternatives and “and/or.”

Additionally, the terms “herein,” “above,” and “below,” and words of similar import, when used in this application, shall refer to this application as a whole and not to any particular portion of the application.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

All references cited herein, whether supra or infra, including, but not limited to, patents, patent applications, and patent publications, whether U.S., PCT, or non-U.S. foreign, and all technical and/or scientific publications are hereby incorporated by reference in their entirety.

### A. Functionalized Nanoparticles

The present disclosure provides functionalized nanoparticles for the intracellular delivery to a target cell of one or more biologically active molecules that affect or introduce one or more cellular functions and/or activities of the target cell. Thus, the functionalized nanoparticles disclosed herein include: (a) one or more targeting molecules, which include cell membrane-penetrating molecule(s) for penetrating through a mammalian cell membrane (e.g., a plasma membrane, a nuclear membrane, a mitochondrion membrane, a lysosomal membrane, an endosomal membrane, and/or other organelle membrane) and, thereby, facilitating the intracellular delivery of the functionalized nanoparticle and (b) one or more biologically active molecule(s) for affecting or introducing one or more cellular functions such as, for example, normalizing, restoring, regulating, and/or modulating (i.e., stimulating or inhibiting) one or more cellular functions such as, for example, cell maintenance, survival, growth/proliferation, differentiation, and/or death.

Within certain embodiments, the present disclosure provides functionalized nanoparticles having (a) a nanoparticle core; (b) one or more targeting molecules, which include cell membrane-penetrating molecule(s); and (c) one or more biologically active molecule(s) for regulating, modulating, normalizing, and/or restoring one or more cellular function(s), wherein each of said one or more cell membrane-penetrating molecule(s) and each of said biologically active molecule(s) is attached directly to the nanoparticle core.

Within other embodiments, the present disclosure provides functionalized nanoparticles having (a) a nanoparticle core; (b) one or more functional group(s) that are associated with and/or attached directly to the nanoparticle core; (c) one or more targeting molecules, which include cell membrane-penetrating molecule(s); and (d) one or more biologically active molecule(s) for regulating, modulating, normalizing, and/or restoring one or more cellular functions, wherein each targeting molecule and each biologically active molecule is attached to the nanoparticle core via the one or more functional group(s).

Within yet other embodiments, the present disclosure provides functionalized nanoparticles having (a) a nanoparticle core; (b) one or more functional group(s) that are associated with and/or attached directly to the nanoparticle core; (c) first and second crosslinking agent(s) each having first and second functional groups, wherein said first crosslinking agent has a first length and said second crosslinking agent has a second length and wherein each of the crosslinking agent(s) is attached to the nanoparticle via a first functional group; (d) one or more targeting molecules, which include cell membrane-penetrating molecule(s); and (e) one or more biologically active molecule(s) for regulating, modulating, normalizing, and/or restoring one or more cellular functions, wherein each targeting molecule and each biologically active molecule is indirectly attached to the nanoparticle core through a crosslinking agent via a second functional group.

Within further embodiments, the present disclosure provides functionalized nanoparticles having (a) a nanoparticle core; (b) a polymer coating or lipid bilayer that encapsulates the nanoparticle core; (c) one or more functional group(s) that are associated with and/or attached directly to the nanoparticle and/or to the polymer coating or lipid bilayer; (d) one or more targeting molecules, which include cell membrane-penetrating molecule(s); and (e) one or more biologically active molecule(s) for regulating, modulating, normalizing, and/or restoring one or more cellular functions, wherein each cell membrane-penetrating molecule and each biologically active molecule is attached to a functional group on the nanoparticle and/or to a functional group on the polymer coating or lipid bilayer.

Within still further embodiments, the present disclosure provides functionalized nanoparticles having (a) a nanoparticle core; (b) a polymer coating or lipid bilayer that encapsulates the nanoparticle; (c) one or more functional group(s) that are associated with and/or attached directly to the nanoparticle and/or to the polymer coating or lipid bilayer; (d) first and second crosslinking agent(s) each having first and second functional groups, wherein said first crosslinking agent has a first length and said second crosslinking agent has a second length; (e) one or more cell membrane-penetrating molecule(s); and (f) one or more biologically active molecule(s) for regulating, modulating, normalizing, and/or restoring one or more cellular functions, wherein each crosslinking agent is attached to the nanoparticle and/or to the polymer coating or lipid bilayer via a first functional group and wherein each cell membrane-penetrating molecule and each biologically active molecule is attached to a second functional group on the crosslinking agent.

Each of the various aspects of the functionalized nanoparticles that are disclosed herein are discussed in the sections that follow.

A1. Nanoparticle Cores

The functionalized nanoparticles disclosed herein include a central nanoparticle core that may be fabricated from a variety of porous, semi-porous, hollow, and solid materials including, for example, metals (e.g., magnetic (paramagnetic and superparamagnetic) and/or conductive metals), ceramic materials, synthetic materials, insulating materials, and/or biological materials (e.g., gelatin or bovine serum albumin (BSA)) and may be fabricated into a variety of shapes including, without limitation, spheres, spheroids, rods, disks, pyramids, cubes, and cylinders.

As used herein, the term “nanoparticle core” refers to a “core” material that can include either a single crystal (monodisperse nanoparticle cores) or a plurality of crystals (polydisperse nanoparticle cores) of, for example, gold or a magnetic material, such as a metal oxide, including superparamagnetic iron oxide. Metal oxides form crystals of from about 1 nm to about 25 nm, or from about 3 nm to about 10 nm, or about 5 nm in diameter. Magnetic metal oxides can further include cobalt, magnesium, zinc, or mixtures of those metals in addition to iron. As used herein, the term “magnetic” refers to materials of high positive magnetic susceptibility.

As used herein, the term “nanoparticle core” is used interchangeably with the terms “nanoparticle,” “nanostructure,” “nanocrystal,” “nanotag,” and “nanocomponent,” which terms collectively refer to particles, generally metallic or ceramic particles, having at least one dimension that ranges from about 0.5 nm to about 100 nm. It is generally understood in the art that the upper limit on the size of a “nanoparticle” is based, primarily, upon the observation that certain properties, which are distinct from those of a bulk material, typically develop at a critical length of 100 nm or less. According to the IUPAC definition, however, and because other phenomena (e.g., transparency or turbidity, ultrafiltration, stable dispersion, etc.) extend the upper limit, the use of the term nanoparticle can include particles having dimensions up to about 500 nm, or up to about 300 nm, or up to about 200 nm. Nanoparticle cores have an overall size of less than about 200 nm before conjugation to biomolecules. The overall size of the nanoparticle cores is from about 0.5 to 200 nm, or from about 1 to 100 nm, or from about 2 to 50 nm. The polymeric coating can be about 5 to 20 nm thick or more. Size can be determined by laser light scattering, by atomic force microscopy or by other suitable techniques.

As used herein, the term “colloid” refers to a broad range of solid-liquid (and/or liquid-liquid) mixtures, all of which containing distinct solid (and/or liquid) particles that are dispersed to various degrees in a liquid medium. The term is specific to the size of the individual particles, which are larger than atomic dimensions but small enough to exhibit Brownian motion. If the particles are large enough then their dynamic behavior in any given period of time in suspension would be governed by forces of gravity and sedimentation. But, if they are small enough to be colloids, then their irregular motion in suspension can be attributed to the collective bombardment of a myriad of thermally agitated molecules in the liquid suspending medium. Colloids size range (or particle diameter) is generally from about 10⁻⁹ m to about 10⁻⁶ m.

Nanoparticle cores may be isotropic or anisotropic. Anisotropic nanoparticle cores may have a length and a width. In some embodiments, the length of an anisotropic nanoparticle is the dimension parallel to the aperture in which the nanoparticle core was produced. In some embodiments, anisotropic nanoparticle cores can have a diameter (i.e., a width) of 200 nm or less, or a diameter of 100 nm or less, or a diameter of 50 nm or less, or a diameter of 25 nm or less.

Suitable nanoparticle cores for making the functionalized nanoparticles of the present disclosure have a hydrodynamic diameter ranging from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Such nanoparticles are generally available in, or may be produced at, a concentration of from about 10¹⁵ nanoparticles per ml to about 10²⁰ nanoparticles per ml.

The hydrodynamic diameter of a given nanoparticle core is dependent upon the solvent in which it is suspended. For example, nanoparticle cores that are suspended in water generally have larger hydrodynamic diameters than nanoparticle cores that are suspended in phosphate-buffered saline (PBS). Modifications, such as pegylation, can increase the hydrodynamic diameter of a nanoparticle core, and can reduce its zeta potential by reducing the number of negative charges.

Methodology for determining the hydrodynamic diameter of a nanoparticle are well known in the art and described, for example, in U.S. Patent Publication No. 2007/0258907. Hydrodynamic diameter measurements often include a determination of dynamic light scattering (DLS), such as may be achieved with a ZetaPALS dynamic light scattering detector (DLS, Brookhaven instruments Corporation).

The zeta potential (mV) of a nanoparticle may be calculated from its electrophoretic mobility using the Smoluchowski equation:

$$\begin{matrix}\frac{\partial c}{\partial t} = D {∇}^{2} c - \vec{υ} \cdot ∇ c . & \\ wherein & \\ \begin{matrix}c is the variable of interest , \\ D is the diffusivity ( a / k / a diffusion coefficient ) , \\ \vec{υ} is the average velocity at which the n anoparticle is moving , and \\ ∇ represents a gradient .\end{matrix} & \end{matrix}$$

The diameter of a nanoparticle core may also be measured by photon correlation spectroscopy (PCS) or by transmission electron microscopy (TEM). An aqueous drop of a nanoparticle solution (i.e., a nanofluid) can be placed on a carbon coated copper grid, and the excess liquid wicked away. The nanoparticle core may then be visualized under an 80 kV electron beam. Typically, nanoparticle cores are visible, while polymer or lipid coatings are transparent to the electron beam and therefore invisible by TEM.

Nanoparticle cores having desired shapes, sizes, and properties are known to those skilled in the art and are described in the literature. While it is recognized that particle shape and aspect ratio (AR) can affect the physical, optical, and electronic characteristics of nanoparticles, the specific shape, aspect ratio, or presence/absence of internal surface area is not determinative of the suitability of a given nanoparticle core for use in making the presently-disclosed functionalized nanoparticles.

Within certain embodiments, functionalized nanoparticles of the present disclosure may be functionalized magnetic nanoparticles, including functionalized paramagnetic and functionalized superparamagnetic nanoparticles, which are manufactured with paramagnetic and superparamagnetic nanoparticle cores, respectively.

As used herein, the term “paramagnetic nanoparticle core” refers, generally, to a nanoparticle core that comprises a metal oxide or a metal mixed oxide wherein the metal may include any one of, or combination of two or more of, aluminum, barium, beryllium, chromium, cobalt, copper, iron, manganese, magnesium, strontium, zinc, a rare earth metal, or a trivalent metal ion. Other metal species, such as silicon oxide, silver, titanium, and ITO can also be used in the presently disclosed paramagnetic nanoparticle cores.

As used herein, the term “superparamagnetic nanoparticle core” refers to a “paramagnetic nanoparticle core” that becomes magnetized when subjected to an external magnetic field. Exemplified herein are functionalized nanoparticles that employ paramagnetic or superparamagnetic nanoparticles wherein the metal is iron, more specifically, an iron oxide, such as a monocrystalline iron oxide. Thus, “superparamagnetic nanoparticle cores” include “superparamagnetic iron oxide nanoparticle cores,” which are made out of a highly magnetic form of iron oxide (e.g., magnetite, non-stoichiometric magnetite, and gamma-ferric oxide) that has a magnetic moment of greater than about 30 EMU/gm Fe at 0.5 Tesla and about 300 K. When magnetic moment is measured over a range of field strengths, it shows magnetic saturation at high fields and lacks magnetic remanence when the field is removed. Certain monocrystalline iron oxide nanoparticle cores, for example, are superparamagnetic at a diameter range from about 3 nm to about 20 nm.

Nanoparticle cores (1) can be encapsulated with a coating, such as a polymer coating and/or a lipid bilayer and (2) can include one or more functional groups, such as one or more amino groups and/or one or more carboxy groups, for attaching (a) one or more cross-linking agent(s), in particular one or more bi-functional cross-linking agent(s), (b) one or more cell membrane-penetrating molecules, and/or (c) one or more biologically active molecules for introducing a new functionality into a target cell and/or for affecting one or more cellular functions of a target cell. Each of these aspects of the present disclosure is described in further detail elsewhere herein.

Exemplary suitable superparamagenetic iron oxide nanoparticle cores (SPIONs) that may be used in the manufacture of the functionalized nanoparticles of the present disclosure include ferumoxides and ferucarbotran, which are encapsulated with dextran or carboxydextran, respectively. Ferumoxide and ferucarbotran nanoparticle cores has been approved for use in in vivo clinical applications, including magnetic resonance imaging (MRI). See, Wang, Quantitative Imaging in Medicine and Surgery 1(1):35-40 (2011).

Ferumoxide nanoparticle cores are available commercially as Feridex IV (Berlex Laboratories), Endorem (Guerbet), and AMI-25 (AMAG Pharma). Feridex is a SPION colloid having a low molecular weight dextran coating and a particle size of 120-180 nm.

Ferucarbotran nanoparticle cores are available commercially as Resovist (Bayer Healthcare) and SH U 555A (Schering AG). Ferucarbotran is a carboxydextran-coated SPIONs having a hydrodynamic diameter ranging between 45 and 60 nm. Ferumoxtran-10 (AMI-227) is available from AMAG Pharma (Combidex) and Guerbet (Sinerem). Clariscan (PEG-fero; Feruglose; NC100150) is manufactured by GE Healthcare.

Paramagnetic and superparamagnetic nanoparticle cores for use in the manufacture of certain of the functionalized nanoparticles disclosed herein may be made according to, or by modification of, methodologies that are known and readily available in the art. See, for example, U.S. Patent Publication No. 2013/0195767, which describes magnetic nanoparticle cores made by thermal decomposition of metal complexes in an oxygen-free environment (e.g., under vacuum or nitrogen environment) and in a solution containing a surfactant.

Several methodologies have been described for synthesizing nanoparticle cores, including both attrition and pyrolysis methodologies. In attrition, macro- or micro-scale particles are ground in a ball mill, a planetary ball mill, or other size-reducing mechanism. The resulting particles are air classified to recover nanoparticles. In pyrolysis, a vaporous precursor (liquid or gas) is forced through an orifice at high pressure and burned. The resulting solid is air-classified to recover oxide particles from by-product gases. Pyrolysis often results in aggregates and agglomerates rather than single primary particles.

A thermal plasma can be employed to provide the energy necessary to cause vaporization of small micrometer-size particles. The thermal plasma temperatures are in the order of 10,000 K, easily evaporating solid powder. Nanoparticle cores are formed upon cooling while exiting the plasma region. Suitable thermal plasma torches for use in producing nanoparticle cores include DC plasma jet, DC arc plasma, and radio frequency (RF) induction plasmas.

In arc plasma reactors, the energy necessary for evaporation and reaction is provided by an electric arc formed between an anode and a cathode. For example, silica sand can be vaporized with an arc plasma at atmospheric pressure. The resulting mixture of plasma gas and silica vapor can be rapidly cooled by quenching with oxygen, thus ensuring the quality of the fumed silica produced.

In RF induction plasma torches, energy coupling to the plasma is accomplished through the electromagnetic field generated by the induction coil. The plasma gas does not come in contact with electrodes, thus eliminating possible sources of contamination and allowing the operation of such plasma torches with a wide range of gases including inert, reducing, oxidizing, and other corrosive atmospheres. The working frequency is typically between 200 kHz and 40 MHz. Laboratory units run at power levels in the order of 30-50 kW, whereas large-scale industrial units have been tested at power levels up to 1 MW. As the residence time of the injected feed droplets in the plasma is very short, it is important that the droplet sizes are small enough to obtain complete evaporation. The RF plasma method has been used to synthesize different nanoparticle materials, for example various ceramic nanoparticle cores such as oxides, carbors/carbides, and nitrides of Ti and Si.

Inert-gas condensation is frequently used to make nanoparticle cores from metals with low melting points. The metal is vaporized in a vacuum chamber and then supercooled with an inert gas stream. The supercooled metal vapor condenses into nanometer-size particles, which can be entrained in the inert gas stream and deposited on a substrate or studied in situ.

Nanoparticle cores can also be formed using radiation chemistry. This technique uses water, a soluble metallic salt, a radical scavenger (e.g., a secondary alcohol), and a surfactant. High gamma doses on the order of 10⁴ Gray are required. Radiolysis from gamma rays can create strongly active free radicals in solution. By this methodology, reducing radicals drop metallic ions down to the zero-valence state. A scavenger chemical preferentially interacts with oxidizing radicals to prevent re-oxidation of the metal. Once in the zero-valence state, metal atoms begin to coalesce into particles. A chemical surfactant surrounds the particle during formation and regulates its growth. In sufficient concentrations, the surfactant molecules stay attached to the particle. This prevents it from dissociating or forming clusters with other particles. Formation of nanoparticle cores using the radiolysis method allows for tailoring of particle size and shape by adjusting precursor concentrations and gamma dose.

The sol-gel methodology is a wet-chemical technique (also known as chemical solution deposition) widely used recently in the fields of materials science and ceramic engineering. Such methods are used primarily for the fabrication of materials (typically a metal oxide) starting from a chemical solution (sol, short for solution), which acts as the precursor for an integrated network (or gel) of either discrete particles or network polymers.

Typical precursors are metal alkoxides and metal chlorides, which undergo hydrolysis and polycondensation reactions to form either a network “elastic solid” or a colloidal suspension (or dispersion)—a system composed of discrete (often amorphous) submicrometer particles dispersed to various degrees in a host fluid. Formation of a metal oxide involves connecting the metal centers with oxo (M-O-M) or hydroxo (M-OH-M) bridges, therefore generating metal-oxo or metal-hydroxo polymers in solution. Thus, the sol evolves toward the formation of a gel-like diphasic system containing both a liquid phase and solid phase whose morphologies range from discrete particles to continuous polymer networks.

In the case of colloids, the volume fraction of particles (or particle density) may be so low that a significant amount of fluid may need to be removed initially for the gel-like properties to be recognized. This can be accomplished by allowing time for sedimentation to occur and then pouring off the remaining liquid. Centrifugation can also be used to accelerate the process of phase separation.

Removal of the remaining liquid (solvent) phase requires a drying process, which is typically accompanied by significant shrinkage and densification. The rate at which the solvent can be removed is ultimately determined by the distribution of porosity in the gel. The ultimate microstructure of the final component will thus be strongly influenced by changes implemented during this phase of processing. Afterward, a thermal treatment, or firing process, is often necessary in order to favor further polycondensation and enhance mechanical properties and structural stability via final sintering, densification, and grain growth. Advantageously over more traditional processing techniques, this methodology can achieve densification at a much lower temperature.

The precursor sol can be either deposited on a substrate to form a film (e.g., by dip-coating or spin-coating), cast into a suitable container with the desired shape (e.g., to obtain monolithic ceramics, glasses, fibers, membranes, aerogels), or used to synthesize powders (e.g., microspheres or nanospheres). The sol-gel approach is an inexpensive and low-temperature technique that allows for the fine control of the product's chemical composition. Even small quantities of dopants, such as organic dyes and rare earth metals, can be introduced in the sol and end up uniformly dispersed in the final product. It can be used in ceramics processing and manufacturing as an investment casting material, or as a means of producing very thin films of metal oxides for various purposes. Sol-gel derived materials have diverse applications in optics, electronics, energy, space, (bio)sensors, medicine (e.g., controlled drug release) and separation (e.g., chromatography) technology.

Paramagnetic nanoparticle cores may be formed by heating a solution comprising a surfactant (e.g., 1-octadecene, oleylamine, oleyamine, and oleic acid) to 250° C. followed by the addition of a metal complex such as, for example, Fe(CO)₅, Fe(acetylacetonate)₂, Fe(acetylacetonate)₃, a cobalt complex, or a nickel complex. The resulting paramagnetic nanoparticle cores, which precipitate out of solution, may then be collected and used for the preparation of the functionalized paramagnetic nanoparticles disclosed herein. This methodology may be modified to form paramagnetic nanoparticle cores comprising one or more functional groups, such as one or more amine groups or carboxylic acid groups, by incorporating chemicals containing one or more of those functional groups into the surfactant solution (further described elsewhere herein).

The size of a nanoparticle core depends, in part, on the molar ratio of metal complex and surfactant. Generally, decreasing levels of surfactant to metal complex ratio increases the size of the resulting nanoparticle core. For example, iron nanoparticle cores having particle diameters of approximately 2 nm may be formed with an oleylamine solution in combination with an iron metal complex at a 1:12 molar ratio of oleylamne:iron. Nanoparticle core size also depends on the reaction temperature during nanoparticle core formation, with particle size increasing as a function of temperature. For example, at a fixed surfactant:iron molar ratio, iron nanoparticle cores of approximately 4.4 nm are formed at 140° C. while iron nanoparticle cores of approximately 14.5 nm are formed at 260° C.

A solution containing a metal complex may be added to a solution containing magnetic, paramagnetic, and/or superparamagnetic nanoparticle cores. To prevent a natural amorphous ferrite shell from forming prior to fabrication of a synthetic shell, the solution containing nanoparticle cores may be maintained in an oxygen-free environment (e.g., under vacuum or primarily nitrogen environment) followed by annealing the mixture of metal complex and nanoparticle core to form a nanoparticle shell on a surface of the nanoparticle core.

In one example, a solution of iron-oleate complex can be prepared from a solution containing Fe(CO)₅, oleic acid, and ODE and annealed under oxygen-free conditions and then combined with a solution containing magnetic iron nanoparticle cores. A synthetic polycrystalline ferrite (Fe₃O₄) shell, which exhibits superparamagnetic properties, forms around the magnetic iron nanoparticle core as the temperature of the mixture increases to 300° C.

Synthetic shells prepared in this manner are stable, maintain a constant thickness over time, and serve as a barrier to prevent oxidation of the nanoparticle core. The magnetic properties of the magnetic iron nanoparticle core may be enhanced by including one or more additional metal complexes such as, for example, Ni(CO)₄, CO₂(CO)₈, or Mn₂(CO)₁₀ in the synthetic shell.

Within further embodiments, the present disclosure provides functionalized nanoparticles comprising gold nanoparticle cores, which exhibit certain advantages, including a high degree of biocompatibility and reduced cytotoxicity, as compared to paramagnetic and superparamagnetic nanoparticle cores, which are described herein or otherwise available in the art. Various applications for gold nanoparticles in intracellular drug delivery are described in Li et al., EnViron. Sci. Technol. 36:405-431 (2006) and Tomar and Garg, Global J. Pharmacol. 7:34-38 (2013).

Colloidal gold has a high affinity for sulfur compounds (thiols or —S—S-compounds). It has been reported that the reaction of —S—H for gold may be enhanced with increasing pH. Exemplary suitable gold nanoparticle cores include the 32 nm nanoparticle cores described by Cytimmune, which have been labeled with Tumor Necrosis Factor (TNF) and Polyethylene Glycol (PEG) or with Taxol and PEG and are being tested in clinical and preclinical trials by Cytimmune (Rockville, Md.) and AstraZeneca (Cambridge, UK). Gold nanoparticle cores labeled with Interferon (CYT-61000) and Gemcitabine (CYT-71000) have also been disclosed by Cytimmune.

The functionalized gold nanoparticles disclosed herein may be used advantageously to enhance the biodistribution and localized concentration of biologically active molecules that are delivered to diseased organs, tissues, or cells and may be employed to deliver unstable biologically active molecules to in vivo sites that are traditionally difficult to access such as, for example, brain and retina tissues, tumors, and intracellular organelles.

It will be understood that the performance of functionalized gold nanoparticles depends upon particle size and surface functionality. The release of biologically active molecules and subsequent nanoparticle disintegration will vary and optimal delivery of a functionalized gold nanoparticle requires availability of the biologically active molecule at the site of action for an appropriate duration and concentration.

Gold nanoparticle cores may be formed in a variety of sizes of hydrodynamic diameter ranging from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm and can be controlled during formation and gold nanoparticle cores are amenable to functionalization with a variety of reactive groups. Gold nanoparticle cores exhibit a maximum rate of cellular uptake in a size range of from 20 nm to 50 nm although cell toxicity has been reported with gold nanoparticle cores in the size range of from 40 nm to 50 nm.

Gold nanoparticle cores, including spherical gold nanoparticle cores, gold nanorods, gold nanowires, and palladium coated gold nanoparticle cores, which may be employed in the manufacture of the functionalized nanoparticles of the present disclosure are available commercially from Nanopartz Inc. (Loveland, Colo.) or may be prepared following protocols as discussed herein or as otherwise known in the art.

A particular advantage of gold, and other speciated metals, over magnetic nanoparticle cores is the desirable property that gold nanoparticle cores can attach directly to chemical or biological entities. Specifically, gold is reactive with sulfhydryl functional groups, which feature may be exploited to attach biologically active molecules and targeting molecules, such as cell membrane-penetrating molecules, including biologically active proteins, polypeptides, and peptides and cell membrane penetrating proteins, polypeptides, and peptides. The size of sulfhydryl conjugated gold nanoparticle cores depends upon the thiol:gold ratio, with higher thiol:gold ratios yielding smaller nanoparticle core sizes.

Due to their tunable size, gold nanoparticle cores can be adapted for the delivery of biologically active molecules, including biologically active proteins, polypeptides, peptides, and nucleic acids, including DNAs and RNAs, such as siRNAs. Fan et al., Colloids Surf, B Bio interfaces 28:199-207 (2003). Gold nanoparticle cores offer other favorable functionalities, such as functionalization with cationic 4° ammonium group, an ability to bind DNA plasmids thorough electrostatic interactions, and an ability to protect DNA from enzymatic digestion.

Gold nanoparticle cores can also work as carriers for peptides and protein. It has been reported, for example, that cationic tetra alkyl ammonium functionalized gold nanoparticles preferentially bind to cell surface receptors. Duncan et al., J. Controlled Release 148:122-127 (2010).

Biotin labels may be attached to gold nanoparticle cores by reaction with a bis-biotin disulfide species. This feature of gold nanoparticle cores may be exploited to prepare functionalized gold nanoparticles of the present disclosure through the attachment via a streptavidin linkage of one or more biologically active molecule(s) and one or more cell membrane-penetrating molecule(s) to a gold nanoparticle core. The resulting size of such functionalized gold nanoparticles depends upon the streptavidin crosslinking, which can be controlled by concentration of either the gold-biotin species or the streptavidin.

Dextran can also be attached to gold nanoparticle cores in a speciation reaction with a solution containing dextran and reducing agent as described herein. Placing dextran onto the gold nanoparticle core requires speciation in solution in the presence of dextran and a reducing agent.

Methodology for the synthesis of gold nanoparticle cores is described in Low and Bansal, Biomedical Imaging and Intervention J. 6:1-9 (2010). Generally, gold nanoparticle cores are produced in a liquid by reduction of chloroauric acid (H[AuCl₄]). After dissolving H[AuCl₄], the solution is rapidly stirred while a reducing agent is added. This causes Au³⁺ ions to be reduced to neutral gold atoms. As gold atoms accumulate, the solution becomes supersaturated, and gold precipitates in the form of sub-nanometer particles. The remainder of the free gold atoms adhere to these particles, and, with vigorous stirring, particles form at uniform size. To prevent particle aggregation, a stabilizing agent that adheres to the nanoparticle surface may be added or laser ablation in liquid may be employed.

The Turkevich reaction produces spherical gold nanoparticle cores, with networks of gold nanowires formed as a transient intermediate. Turkevich and Kim, Science 169(3948):873-9 (1970). The Frens methodology, which involves the reaction of hot chlorauric acid with sodium citrate generates monodisperse spherical gold nanoparticles having diameters of approximately 10-20 nm. Frens, Nature 241:20 (1973). Colloidal gold forms in the presence of citrate ions, which act as both a reducing agent and a capping agent. Larger particles can be produced by decreasing sodium citrate concentration, which reduces the availability of citrate ions for stabilizing the nanoparticles. As a result, small nanoparticle cores form larger aggregates having a reduced surface area, which permits saturation of the surface with citrate ions. See, also, Jana et al., Advanced Materials 13(18):1389-93 (2001), Perrault and Chan, J American Chemical Society 131(47):17042-3 (2009), and McDaniel and Astruc, Chemical Reviews 104(1):293-346 (2004).

The Brust and Schiffrin methodology produces gold nanoparticle cores of from 5 nm to 6 nm in organic liquids, such as toluene, which are normally not miscible with water. A chlorauric acid solution is reacted with a phase transfer catalyst and stabilizing agent, such as tetraoctylammonium bromide (TOAB) solution in toluene, in the presence of a reducing agent, such as sodium borohydride. See, Brust et al., J Chem Soc Comm 7:801-802 (1994); Brust et al., Langmuir 14(19):5425-9 (1998); and Brust et al., J Chem Soc Comm 16:1655-6 (1995).

Because TOAB does not bind with high affinity to gold nanoparticle cores, the reaction requires gradual aggregation over the course of approximately two weeks. This process may be accelerated with the addition of a higher affinity gold-binding agent, like a thiol, such as an alkanethiol. Alkanethiol-protected gold nanoparticle cores can be precipitated and then redissolved, however some phase transfer agent may remain bound to the purified nanoparticle cores, which may affect physical properties including solubility. Remaining phase transfer agent may be removed with a Soxhlet extraction purification step.

Gold nanoparticle cores may be prepared by hydroquinone reduction of HAuCl₄ in an aqueous solution containing gold nanoparticle seeds as described in Perrault and Chan, J. American Chemical Society 131(47):17042-3 (2009). This seed-based method is analogous to the methodology used in photographic film development wherein silver grains within the film grow through addition of reduced silver onto their surface. Similarly, gold nanoparticle cores act in conjunction with hydroquinone to catalyze the reduction of ionic gold onto the nanoparticle core surface. A stabilizer, such as citrate, can be added to control particle growth.

The Perrault and Chan hydroquinone methodology complements the Frens methodology by extending the range of monodispersed spherical nanoparticle core sizes that can be produced. While the Frens chlorauric acid methodology generates gold nanoparticle cores in the range of approximately 12 nm to 20 nm, the Perrault and Chan hydroquinone methodology produce nanoparticle cores in the range of approximately 30 nm to 250 nm.

Precise size control with a low polydispersity of spherical gold nanoparticle cores remains difficult for particles larger than 30 nm. To maximize control over nanoparticle core structure, Navarro and co-workers developed a modified Turkevitch-Frens procedure, which incorporates sodium acetylacetonate (Na(acac)) to reduces AuIII to AuI, which may be further reduces to Au0 by the addition of sodium citrate. The concentration of Na(acac) determines nuclei number and produces gold cores of up to 90 nm with a narrow size range distribution. Tetrachloroaurate and sodium citrate concentrations are fixed to 0.30 mM and 0.255 mM (to achieve a sodium citrate:gold ratio of 0.85). Na(acac) may be used at a concentration of from 0.33 mM to 1.0 mM and, in the presence of sodium citrate, reduces AuIII to AuI and results in the formation of gold nuclei, which diffuse over the solution to yield the final spherical particles.

Gold nanoparticle cores may also be prepared by a sonolysis methodology, which employs ultrasound to promote the reaction of an aqueous solution of HAuCl₄ with glucose and the production of hydroxyl radicals and sugar pyrolysis radicals as reducing agents. Okitsu et al., Bulletin Chemical Society Japan 75(10):2289-96 (2002),Vinodgopal et al., J. Physical Chemistry Letters 1(13):1987-93 (2010), and Okitsu et al., J. Phys Chem B 109(44):20673-20675 (2005). Gold nanoparticle cores generated by the sonolysis methodology have a nanoribbon morphology with a width in the range of about 30 nm to about 50 nm and a length of several μm. These ribbons are very flexible and can bend with angles larger than 90°. Glucose may be replaced with a glucose oligomer, such as cyclodextrin, to form spherical gold particles.

A block copolymer methodology for generating gold nanoparticle cores uses block copolymer as both a reducing agent and a stabilizing agent. Alexandridis, Chemical Eng & Tech 34(1):15-28 (2011) and Kang and Taton, Angewandte Chemie 117(3):413-6 (2005). Gold nanoparticle cores are formed in three steps: (1) gold clusters are formed by reduction of gold salt ions in solution with block copolymers, (2) block copolymers are adsorbed onto gold clusters and gold salt ions are further reduced on the gold cluster surfaces to achieve the stepwise growth of gold nanoparticles, and (3) the gold nanoparticles are stabilized by further addition of block copolymers. A reductant, such as trisodium citrate, may be added in a 1:1 molar ratio with gold salt to enhance the yield of gold nanoparticle cores.

Within certain embodiments, the present disclosure provides functionalized nanoparticles having biocompatible and biodegradable polymeric composite nanoparticle cores, including biocompatible and biodegradable poly-lactic acid/poly-glycolic acid (PLGA) nanoparticle cores. Within certain aspects of these embodiments one or more cell targeting molecule(s) and/or one or more biologically active molecule(s) are covalently attached to a polymeric composite nanoparticle core. Within other aspects of these embodiments, one or more cell targeting molecule(s) and/or one or more biologically active molecule(s) are entrapped within a polymeric composite nanoparticle core.

Exemplary biocompatible and biodegradable polymeric composite nanoparticle cores that may be used in the manufacture of the functionalized nanoparticles disclosed herein are known in the art. U.S. Pat. No. 8,003,128 describes poly(DL-lactide) and poly(DL-lactide-co-glycolide) nanoparticle cores for administering pharmacologically active substances across a mammalian blood brain barrier and, thereby, deliver the active substances to the central nervous system. U.S. Patent Publication No. 2015/0283095 describes biodegradable and biocompatible polymer nanoparticle cores that are manufactured with poly(lactic-glycolic) acid (PLGA) for delivering the drug pentoxifylline.

Biocompatible and biodegradable polymeric composite nanoparticle cores that may be used in the manufacture of the functionalized nanoparticles disclosed herein are also available commercially. For example, Phosphorex Inc. (Hopkinton, Mass.) manufactures polystyrene, PLGA, and PMMA nanoparticle cores (i.e., nanospheres) in sizes ranging from 20 nm to 200 nm, including nanospheres in sizes ranging from 50 nm to 100 nm. Such nanoparticle cores may be coated with, for example, Tween-80 (a/k/a polysorbate-80). Cell targeting molecules, including cell membrane penetrating molecules, and biologically active molecules may be attached to the surface of these nanoparticle cores either via covalent interactions, physical adsorption, or intercalation within a nascent core as the nanoparticle core is formed to, thereby, produce functionalized nanoparticles that are suitable for intracellular delivery, targeted drug delivery, and for applications requiring transport across a mammalian blood-brain barrier.

Within certain aspects, such biocompatible and biodegradable polymeric composite nanoparticle cores may be coated with amidated polysorbate-80, which permits the linking of cell targeting molecules and biologically active molecules to the surface of a nanoparticle core and, thereby, facilitates the cell specific targeting and intracellular delivery of biologically active molecules. See, also, Lim et al., Biotechnology Progress 26(6):1528-33 (2010) and Kuno and Fuji, Polymers 3(1):193-221 (2011).

A2. Functional Groups

Functionalized nanoparticles according to certain embodiments of the present disclosure employ nanoparticle cores comprising one or more functional groups that are associated with or directly attached to the nanoparticle core and/or one or more functional groups that are associated with or directly attached to a coating, such as a polymer coating or lipid bilayer, which encapsulates the nanoparticle core.

Such functional groups permit (1) the direct and independent attachment to the nanoparticle core of one or more targeting molecules, including cell membrane-penetrating molecule(s) and/or one or more biologically active molecule(s) for introducing or affecting a cellular function and/or (2) the direct attachment of one or more cross-linking agents (in particular one or more bi-functional cross-linking agents) for the indirect and independent attachment to the nanoparticle core of one or more targeting/cell membrane-penetrating molecule(s) and/or one or more biologically active molecule(s) for introducing or affecting a cellular function.

Suitable functional groups that may be used in the functionalized nanoparticles disclosed herein include, for example, amino groups (—NH₂), sulfhydryl groups (—SH), carboxyl groups (—COOH), guanidyl groups (—NH₂—C(NH)—NH₂), hydroxyl groups (—OH), azido groups (—N₃), and/or carbohydrates. Such functional groups can attach directly to a biologically active molecule, a cell membrane-penetrating molecule, and/or a crosslinking agent through, for example, an amino, sulfhydryl, or phosphate group. Alternatively, a functional group can be provided as a functionalized polymer that is formed, for example, on a synthetic nanoparticle shell.

Functional groups may also include one or more stabilizing groups, such as stabilizing groups selected from the group consisting of phosphate, diphosphate, carboxylate, polyphosphate, thiophosphate, phosphonate, thiophosphonate, sulphate, sulphonate, mercapto, silanetriol, trialkoxysilane-containing polyalkylene glycol s, polyethylene glycols, carbohydrate or phosphate-containing nucleotides, oligomers thereof or polymers thereof.

Methodologies for adding functional groups to nanoparticle cores are described in Halbreich et al., Biochimie 80(5-6).379-90 (1998) and Valois et al., Biomaterials 31(2):366-374 (2010) and are exemplified within the present disclosure. Carboxy-functionalized nanoparticle cores can be converted into amino-functionalized nanoparticle cores by use of water-soluble carbodiimides and diamines, such as ethylene diamine or hexane diamine, Other methodologies for attaching molecules to nanoparticle cores employ the reactivity of an aldehyde group. For example, Rembaum describes nanoparticle cores with glutaraldehyde functional groups. See, e.g., Rembaum et al., Macromolecules 9:328 (1976), Hiremath and Hota, Indian J. Pharma. Sci. 61(2):69-75 (1999), and U.S. Pat. Nos. 4,438,239 and 4,369,226).

U.S. Pat. No. 8,420,055 describes amine functionalized superparamagnetic nanoparticle cores, in particular amine-functionalized crosslinked iron oxide nanoparticle cores (“amino-CLIO”), which can be used according to the methods of the present disclosure to generate the functionalized nanoparticles that are described herein.

Amino-CLIO is prepared by synthesizing a dextran-coated nanoparticle, followed by crosslinking the dextran with epichlorohydrin. Amine groups are incorporated by reacting the crosslinked dextran with ammonia as is described, for example, in Josephson et al., Bioconjug. Chem. 10:186-91 (1999) and Josephson et al., Angwandte Chemie 40:3204-3206 (2001).

Amino-CLIO may be used for the attachment of one or more biologically active molecules and/or targeting/cell membrane-penetrating molecules, either via direct attachment or through one or more crosslinking agents. The amine group can, for example, be reacted with a wide variety of N-hydroxysuccinimide ester-based crosslinkers, which react an amine group on the amino-CLIO and with sulfhydryl groups on one or more biologically active molecules (including peptides, proteins, and nucleic acids) and/or under a wide range of conditions (temperature, pH, ionic strength). Such bifunctional crosslinking agents are described in further detail herein and are exemplified by SPDP, SIA, SMCC, and MBS, each of which is commercially available (e.g., from Pierce Chemical, Rockford Ill. or Molecular Biosciences, Boulder, Colo.). See, also, Josephson et al., Bioconjug. Chem. 10:86-91 (1999); Josephson et al., Angwandte Chemie 40:3204-3206 (2001); and 1-logemann et al., Bioconjug. Chem. 11:941-6 (2000).

The amino-CLIO-based chemistry has one major drawback, which arises due to the extraordinary stability achieved with a crosslinked-stabilized dextran on the nanoparticle surface. For human parenteral applications, such as magnetic labelling of targeted MR contrast agents, degradation or elimination of the agent, including the coating, is required. However, when the iron oxide of an amino-CLIO based MR contrast is dissolved or degraded, the crosslinked dextran remains as a non-degradable sphere of polysaccharide. Similarly, non-degradability occurs with micron-sized magnetic microspheres where iron oxide is entrapped in a non-biodegradable polymeric shell. See, e.g., U.S. Pat. Nos. 4,654,267 and 5,512,439.

A carboxy functionalized surface coating can be formed on a nanoparticle according to the methodology of Gorman, PCT Patent Publication No. WO 2000/061191, wherein reduced carboxymethyl (CM) dextran is synthesized from commercial dextran. CM-dextran and iron salts are mixed together and neutralized with ammonium hydroxide. The resulting carboxy functionalized nanoparticles can be used for coupling via an amino group on a bioactive molecule, a cell-membrane penetrating molecule, and/or a crosslinking agent.

Carboxy functionalized nanoparticles can also be made from polysaccharide-coated nanoparticles by reaction with bromo or chloroacetic acid in a strong base to attach the carboxyl groups or from amino-functionalized nanoparticles by converting the amino groups into carboxy groups using reagents such as succinic anhydride or maleic anhydride.

Nanoparticle functionalization can be achieved by the direct derivatization with functional silanes in a reaction that uses a functional silane reagent with a glass-coated NBC or alternatively in the presence of TEOS, whereby the functional silane is introduced along with a second TEOS treatment. These functionalization routes provide the flexibility to conjugate practically any type of molecule and, moreover, take advantage of the large library of functional PEGS that are available in the art such as, for example, from Nektar (San Francisco, Calif.).

Carboxymethyl (CM)-polymers can also function as starting materials for the synthesis of drug conjugates or for the attachment of various biological molecules. As drug conjugates, CM-arabinogalactan, CM-dextran and polyvinyl alcohol have been used as carriers for nucleotide analogues (U.S. Pat. No. 5,981,507). The carboxyl groups may be converted to primary amino groups by reaction with diamines and biological molecules attached to the primary amine. (See, Josephson et al., Antivir. Ther. 1:147-56 (1996) and U.S. Pat. No. 5,478,576).

In these examples, the CM-polymers, such as CM-arabinogalactan, exist as macromolecules in solution, which allows conditions ensuring the nearly quantitative conversion of carboxyl groups to amino groups. The absence of protected carboxyl groups allows essentially all carboxyl groups to be chemically reactive.

The amine-functionalized nanoparticle cores can be synthesized by activation of free carboxyl groups with a water soluble carbodiimide, followed by reaction with a large excess of a diamine, such as ethylenediamine (EDA), propyldiamine, spermidine, spermine, hexanediamine, and diamine amino acids, such as lysine or ornithine, to provide a linker arm of varying length and chemistry for the attachment of crosslinking agents, bioactive molecules, and or cell membrane-penetrating molecules.

In the synthesis of amino-CLIO, dextran-coated magnetic nanoparticle cores may be reacted with epichlorohydrin, followed by reaction with ammonia. This reaction produces a dextran crosslinked, amine-functionalized nanoparticle bearing primary amino groups (H₂N—CH₂—CHOH—CH₂—O— Polymer). Reaction of a carbodiimide activated carboxylated nanoparticle with ammonia results in the formation of an amide (H₂N—CO—CH₂—C— Polymer). The nitrogen atoms of amides are less reactive than primary amino groups and, generally, not suitable for reaction with bifunctional conjugating reagents (i.e., crosslinking agents) that are used to attach biomolecules, including the biologically active molecules and cell penetrating molecules that are disclosed herein.

The reaction with diamine may be performed using a large excess of diamine to prevent crosslinking between nanoparticles. In general, the moles of diamine used will exceed the number of carboxyl groups present by a factor of at least 10. Unreacted diamine (MW<2 kDa) may be separated from amino functionalized nanoparticles (MW>500 kDa) by ultrafiltration. Alternatives to ultrafiltration for the removal of unreacted diamine include gel permeation chromatography, dialysis, and precipitation and resolubilization of the nanoparticle.

When the carbodiimide-activated carboxylated nanoparticles of the disclosure are reacted with a large excess of diamine, one of the nitrogen atoms reacts with the carboxyl group to provide a peptide bond, while a second nitrogen atom exists as a primary amine suitable for further chemistry, Hence the amino-functionalized nanoparticles of the disclosure have a characteristic general structure that includes a peptidyl bond and a primary amino group. This characteristic structure is not found with amino functionalized amino-CLIO nanoparticles. Similarly, a peptide bond is not obtained when dextran-coated magnetic iron oxides are activated by treatment with periodate, followed by reaction with a primary amine and treatment with a reducing agent. In that case a methyl amine linkage is obtained.

The presence of primary amino groups on magnetic nanoparticles can be readily ascertained by reaction with amine specific reagents such as TNBS, ninhydrin, or SPDP with the intact magnetic nanoparticle. Since the carboxyl groups are protected by the metal oxide, they can be most easily analyzed after digestion of the metal oxide core and isolation of the polymeric coating. Digestion of a metal oxide core may be accomplished by treatment with acid and chelator—typically at a pH below 5 or between pH 2 and pH 5. Chelators (e.g., citrate or EDTA) enhance the solubility of iron and may be added in an amount sufficient to bind all metal ions. After digestion, the metal can be removed by passage over a cation exchange column or metal-removing chelating column such as Chelex. The polymer may then analyzed by IR, which reveals characteristic peaks from carboxyl groups. Polymers with carboxyl groups have characteristic absorption frequencies from the carbonyl group (C═O) of the carboxyl (1780 to 1710 cm-1, strong) and the hydroxyl group (3000 to 2500 cm-1, broad, variable).

Depending upon the structural composition and biological reactivity of the nanoparticle core used to manufacture functionalized nanoparticles according to the present disclosure, it may be advantageous to encapsulate the nanoparticle core with a polymer coating, such as a crosslinked or non-crosslinked polymer coating, or with a lipid bilayer.

The encapsulation of nanoparticles with various coatings is described in the literature and well known to those of skill in the art. See, e.g., Petri-Fink et al., Biomaterials 26(15):2685-94 (2005) for a description of SPIO nanoparticle cores coated with polyvinyl alcohol (PVA), carboxylate-functionalized PVA, thiol-functionalized PVA, and amino-functionalized PVA (amino-PVA); U.S. Pat. No. 6,123,920 for a description of methodology for encapsulating SPION with an oxidatively cleaved starch coating optionally with a functionalized polyalkyleneoxide to prolong blood resistance; PCT Patent Publication No. WO 2012/169973 for a description of nanoparticles encapsulated by a polymeric shell; U.S. Patent Publication No. 2004/0265233 for a description of methods for producing superparamagnetic iron oxide nanoparticles that are encapsulated with a polysiloxane (SiO2) matrix containing functional groups.

Functionalized nanoparticles of the present disclosure can also further include one or more chelators, radioisotopes, and/or contrast agents. Representative chelators can be selected from the group consisting of 1,4,7,10-tetraazacyclododecane-N,N′,N″,N′″-tetraacetic acid (DOTA), tetra-butyl-calix[4]arene-crown-6-dicarboxylic acid (TBBCDA), 5,11,17,23-tetra-t-butyl-25,26,27,28-tetrakis (carboxymethoxy)-calix[6]arene (HBHC), diethylenetriamine-pentaacetic acid (DTPA) EDTA, and combinations thereof.

Representative radioisotopes can be selected from the group consisting of yttrium-90, indium-111, radium-23, actinium-225, bismuth-212, bismuth-213, scandium-47, astatine-211, rhenium-186, rhenium-188, iodine-131, iodine-124 lutetium-177, holmium-166, samarium-153, copper-64, copper-67, phosphorus-32, and combinations thereof.

Representative carrier ligands can include gamma-emitting radioisotopes, such as one or more gamma-emitting radioisotopes that are selected from the group consisting of arsenic-74, copper-64, copper-67, fluorine-18, gallium-67, indium-111, iodine-131, rhenium-186, rhenium-188, technetium-99m, thorium-201, yttrium-86, yttrium-91, zirconium-89, and combinations thereof.

A3. Coatings for Encapsulating Nanoparticle Cores

Within certain embodiments, nanoparticle cores may be encapsulated with a polymer coating or a lipid bilayer to (1) reduce nanoparticle cytotoxicity, (2) increase nanoparticle hydrophilicity or hydrophobicity, and/or (3) to provide a surface that can be modified with one or more functional groups for attachment to one or more crosslinking agents, biologically active molecules, and/or cell membrane-penetrating molecules.

Coatings may include, prior to use in encapsulating the nanoparticle core, a functional group. Functional groups include, for example, one or more reactive carboxyl groups and/or one or more reactive primary amino groups that facilitate the attachment of a crosslinking agent, a membrane penetrating molecule, and/or a biologically active molecule as described in detail elsewhere herein). Alternatively, a functional group may be attached to a polymer coating after the nanoparticle is encapsulated.

One or more functional groups, which can be in the form of a functionalized polymeric or non-polymeric coating, can be added to a nanoparticle surfactant to facilitate the direct attachment of a biologically active molecule and/or a cell membrane-penetrating molecule or, alternatively, the indirect attachment of a biologically active molecule and/or a cell membrane-penetrating molecule through a crosslinking agent.

As used herein, the terms “coat” or “coating” refer to complete or partial non-covalent association of, for example, a polymer or lipid bilayer with the surface of a nanoparticle core. As used herein, the term “polymer coating” refers to a linear or branched, natural or synthetic polymer that is associated with and encapsulates a nanoparticle core. A polymer coating can be a continuous film around a nanoparticle or can be a “mesh” or “cloud” of extended polymer chains attached to and surrounding the nanoparticle. A polymer can include one or more functional groups such as an amino group or a carboxy group, such as a polymer coating of carboxy dendrimers (Sigma-Aldrich, St. Louis, Mo.), which are highly branched polycarboxyl polymers.

The term “cyclodextrin moiety” refers to (α, β, or γ) cyclodextrin molecules or derivatives thereof, which may be in their oxidized or reduced forms. Cyclodextrin moieties may comprise optional linkers. Optional therapeutic agents and/or targeting ligands may be further linked to these moieties via an optional linker. The linkage may be covalent (optionally via biohydrolyzable bonds, esters, amides, carbamates, and carbonates) or may be a host-guest complex between the cyclodextrin derivative and the therapeutic agent and/or targeting ligand or the optional linkers of each. Cyclodextrin moieties may further include one or more carbohydrate moieties, preferably simple carbohydrate moieties such as galactose, attached to the cyclic core, either directly (i.e., via a carbohydrate linkage) or through a linker group.

Upon copolymerization of a crosslinking agent with a cyclodextrin monomer precursor, two cyclodextrin monomers may be linked together by joining the primary hydroxyl side of one cyclodextrin monomer with the primary hydroxyl side of another cyclodextrin monomer, by joining the secondary hydroxyl side of one cyclodextrin monomer with the secondary hydroxyl side of another cyclodextrin monomer, or by joining the primary hydroxyl side of one cyclodextrin monomer with the secondary hydroxyl side of another cyclodextrin monomer. Accordingly, combinations of such linkages may exist in the final copolymer. The linker group may be neutral, cationic (e.g., by containing protonated groups such as, for example, quaternary ammonium groups), or anionic (e.g., by containing deprotonated groups, such as, for example, sulfate, phosphate, borinate or carboxylate). The charge of the linker group may be adjusted by adjusting pH conditions, Examples of suitable linker groups include, but are not limited to, succinimide (e.g., (dithiobis(succinimidyl propi onate) (DSP)) and di ssucinimidyl suberate (DSS)), glutamates, and aspartates.

The cyclodextrin-containing polymers which coat the paramagnetic particle of the present disclosure are preferably linear. As used herein, the term “linear cyclodextrin-containing polymer” refers to a polymer comprising (α, β, or γ) cyclodextrin molecules, or derivatives thereof which are inserted within a polymer chain. The term “graft polymer” as used herein refers to a polymer molecule which has additional moieties attached as pendant groups along a polymer backbone. The term “graft polymerization” denotes a polymerization in which a side chain is grafted onto a polymer chain, which side chain comprises one or several other monomers. The properties of the graft copolymer obtained such as, for example, solubility, melting point, water absorption, wettability, mechanical properties, adsorption behavior, etc., deviate more or less sharply from those of the initial polymer as a function of the type and amount of the grafted monomers. The term “grafting ratio,” as used herein, means the weight percent of the amount of the monomers grafted based on the weight of the polymer.

The surface coating encapsulating a nanoparticle core can alter the properties of a functionalized nanoparticle by affecting its stability, solubility, and/or targeting. For example, multivalent or polymeric coatings can be employed to substantially increase nanoparticle stability. Functionalized nanomaterial-based catalysts can also be used to catalyze a wide variety of organic reactions. For biological applications, surface coatings may be polar to enhance aqueous solubility and to reduce nanoparticle aggregation. In serum or on the surface of a cell, highly charged coatings can promote non-specific binding, whereas polyethylene glycol linked to terminal hydroxyl or methoxy groups reduces non-specific interactions.

Natural polymers include macromolecules such as proteins, DNAs, RNAs, synthetic polyaminoacids (e.g., polylysine or polyglutamic acid), carbohydrates (e.g., dextran, pullanan, carboxydextran, carboxymethyl dextran, and reduced carboxymethyl dextran, polymethylmethacrylate polymers and polyvinyl alcohol polymers), and lipids.

Synthetic polymers, such as polyethylene glycol, silane, polymethylmethacrylate, block copolymer dendrimer, polyamide, polyethylenimine, polyacrylate, and polyvinyl alcohol, can be obtained from nonbiological syntheses, by using standard polymer chemistry techniques that are known to those having skill in the art.

Polymers can be homopolymers, which are synthesized from a single monomeric unit, or can be co-polymers that are synthesized from two or more monomeric units. Crosslinked polymers are those in which one or more functional groups on a polymer chain reacts with functional groups on another polymer chain to form a polymer network. Crosslinked polymers typically exhibit increased temperature stability and are resistant to degradation in vivo. The molecular weight of a crosslinked polymer is substantially higher than that of a non-crosslinked polymer.

Depending upon the precise application contemplated, a coating can be applied after functionalization of a nanoparticle core or, if a functional group will be attached to and/or associated with the coating itself, the coating can be applied directly to a non-functionalized nanoparticle core.

Polymers and other coatings that are used to manufacture the functionalized nanoparticles of the present disclosure must non-toxic if those functionalized nanoparticles are to be administered for diagnostic and/or therapeutic benefit to a human and must also form non-toxic degradation products as the polymer degrades in vivo. Thus, polymers, lipid bilayers, and other coatings that are employed in the functionalized nanoparticles of the present disclosure may be “biocompatible,” meaning that the polymer, lipid bilayer, and/or other coating is not toxic to a host (i.e., a human or other mammal) and does not degrade at a rate that produces monomeric or oligomeric subunits or other byproducts at toxic concentrations in vivo.

Polymer coatings for in vivo therapy can, for example, biodegrade within a period of less than about five years, less than about one year, less than about six months, less than about three months, less than about one month, less than about fifteen days, less than about five days, less than about three days, or less than about one day upon exposure to a physiological fluid with a pH of from 5 to 9, a pH of from 5.5 to 8.5, or a pH from 6 to 8, or a pH of about 7 at a temperature of from 35° C. to 39° C., or from 36° C. to 38° C., or about 37° C.

Some synthetic biodegradable polymers yield oligomers and monomers in vivo, which can adversely interact with the surrounding tissue. See, for example, Williams, Mater. Sci. 1233 (1982). To minimize the toxicity of an intact polymer and its degradation products, suitable polymers can be employed that are based upon naturally-occurring metabolites, such as polysaccharides, including dextrans and other carbohydrates; polyesters, such as those derived from lactic or glycolic acid; and polyamides, such as those derived from amino acids. Exemplary biodegradable polymers, which are well known and readily available in the art include those used for controlled release of pharmaceuticals, such as those described in U.S. Pat. Nos. 4,291,013; 4,347,234; 4,525,495; 4,570,629; 4,572,832; 4,587,268; 4,638,045; 4,675,381; 4,745,160; and 5,219,980.

The toxicity of a nanoparticle coating that is intended for in vivo use, such as implantation or injection into a patient, can be determined by methodologies that are well known and readily available in the art such as, for example, assay systems that include live mammalian cells in culture, which are contacted with samples of degraded nanoparticle coatings. Polymer coatings can, for example, be degraded in 1 M MOH at 37° C., until complete degradation is observed. The solution can be neutralized with 1 M HCl prior to applying 200 each of various concentrations of degraded sample products in 96-well tissue culture plates seeded with mammalian cells. The rate of cell growth may be determined by determining the number of live cells as a function of time and concentration of degraded coating. The toxicity of coatings can also be evaluated by well-known in vivo tests, such as subcutaneous implantation into rats, to rule out significant levels of irritation or inflammation at the subcutaneous implantation sites.

Biodegradable polymers, such as polylactic acid, polyglycolic acid, and polylactic-glycolic acid copolymer (PLEA), have been previously described and characterized for use in the preparation of nanoparticle formulations. These polymers are polyesters that undergo simple hydrolysis after in vivo administration to a mammal. The hydrolysis products of such polymers are biologically compatible and metabolizable moieties (e.g., lactic acid and glycolic acid), which are removed from the body through the citric acid cycle. Polymer biodegradation products are formed at a very slow rate and do not affect normal cell function and are FDA-approved for human use.

Biodegradable polymers may contain one or more biohydrolyzable bonds. As used herein, the term “biohydrolyzable bond” refers to a bond that is cleaved under physiological conditions and include, for example, esters, amides, carbonates, carbamates, and imides that can be cleaved (1) in acidic and basic environments, such as within a digestive tract, an acidic environment of a tumor; (2) via an enzyme catalyzed reaction; and/or (3) through normal metabolic processing in the liver.

To be used in the synthesis of polymer-coated nanoparticle cores, unreacted polymer is separated from the polymer coated nanoparticle core. This may be achieved by maintaining the polymer in a homogenous size distribution, which can be determined by the methodology known in the art including light scattedng and gel permeation chromatography. For example, unreacted carboxymethylated dextran (MW 20 kDa) can be readily separated from a coated nanoparticle (MW ^{>}500 kDa) by ultrafiltration using a membrane with a cutoff of 100 kDa. See, e.g., PCT Patent Publication No. WO 2000/061191. Polymers that are larger than about 200 kDa can be used but are generally more difficult to separate from polymer coated nanoparticle cores because both the polymer coated nanoparticle cores and the larger molecular weight polymers pass through a membrane having the same pore size or molecular weight cutoff.

General methodologies for applying a polymer coating to a nanoparticle core are known in the art. See, e.g., Petri-Fink et al., Biomaterials 26(15):2685-94 (2005) (describing SPIO nanoparticles coated with polyvinyl alcohol (PVA), carboxylate-functionalized PVA, thiol-functionalized PVA, and amino-functionalized PVA (amino-PVA)); U.S. Pat. No. 6,123,920 (describing methodology for encapsulating SPION with an oxidatively cleaved starch coating optionally with a functionalized polyalkyleneoxide to prolong blood resistance); PCT Patent Publication No. WO 2012/169973 (describing nanoparticles encapsulated by a polymeric shell); and U.S. Patent Publication No. 2004/0265233 (describing methodology for producing superparamagnetic iron oxide nanoparticles that are encapsulated with a polysiloxane (SiO₂) matrix containing functional groups), each of which is incorporated herein by reference in its entirety.

Uncrosslinked anionic cyclodextrin polymers may be prepared as follows: Cross-linked anionic polymer (1.5 g) is dissolved in methanol (10 mL), followed by the addition of aqueous NaOH (1M, 10 mL). The reaction is stirred for 15 h at room temperature. The methanol is removed by vacuum. The remaining solution is frozen, and the water is removed by lyophilization. The solution is resuspended in water (10 mL) and dialyzed against water using a 7K MWCO dialysis cartridge (Pierce). The polymer solution is lyophilized to remove the water, resulting in a white, fluffy powder.

Cyclodextrin-polymers may be used to coat nanoparticle cores, including iron oxide paramagnetic nanoparticle cores and gold nanoparticle cores as follows. Nanoparticle cores encapsulated in a linear, anionic cyclodextrin polymer, are prepared by an aqueous phase coprecipitation method, similar to a process described in U.S. Pat. No. 5,262,176 for the preparation of iron oxide nanoparticles coated with dextran. To prepare larger 90 nm particles, cross-linked, anionic cyclodextrin polymer (100 kDa) (5.125% w/v) is dissolved in 5 mL of degassed double-distilled H₂O.

Alternatively, to make 30 nm particles, uncrosslinked anionic cyclodextrin polymer* (80 kDa) (6% w/v) is dissolved in degassed water. FeCl₃ (6H₂O)hexahydrate (0.12 M) is added to the cyclodextrin polymer mixture and magnetically stirred under argon. Next, 0.063 g of FeCl₂(7H₂O)_heptahydrate is dissolved in 215 μL of degassed ddH₂0. The FeCl₂ solution is added to the mixture containing cyclodextrin polymer and FeCl₃ such that the final reaction mixture contains a 2:1 molar ratio of Fe³⁺ to Fe²⁺. The solution is cooled to 0-4° C. under argon. An aqueous solution of 28% NH₄OH (225 μL) is added dropwise to the reaction. The solution is heated slowly to 80° C. over 45 minutes, and the temperature is maintained at 80° C. for 75 min. with stirring.

The solution is cooled to room temperature by removing the heat source. After cooling, the solution is spun in a centrifuge at 3200 rpm for 10 min. Precipitated material is discarded, and the supernatant, which contains CD polymer-coated iron oxide particles, is mixed with ammonium citrate buffer pH 8.2 (1 mM and 10 mM for larger particles and smaller particles, respectively) in a 1:1 ratio of buffer to supernatant. To remove excess cyclodextrin polymer and ammonium hydroxide base, the solution is purified by ultrafiltration in an Amicon Ultra 4 MWCO 100K unit and centrifuged at 3200 rpm for 15 min. The concentrate is mixed with an equal volume of ammonium citrate buffer, and the ultrafiltration step is repeated twice. The resulting solution contains iron oxide nanoparticles coated with cyclodextrin polymer in citrate buffer at pH 8.2.

The concentration of iron in nanoparticle cores can be determined by measuring absorbance at 356 nm. The validity of this technique was confirmed by measuring the iron content of the particles by ICP-MS. The concentration of polymer in the final solution is determined by a phenol-sulfuric acid assay. The composition of the final nanoparticle solution is typically 20-25 mg/mL of polymer per 1 mg/mL of iron for the larger F3 particles, and the composition of the final nanoparticle solution is typically 5-10 mg/mL of polymer per 1 mg/mL of iron for the smaller F1 particles.

Polycarboxylated polymers may be generated in a reaction containing a water-soluble polymer containing multiple amino or hydroxyl groups and an alkyl halogenated acid in aqueous strong base. This methodology has several advantages: (1) the size and distribution of the polymer obtained is determined by the size of the starting polymer; by selecting a polymer of uniform size, size homogeneity of the resulting carboxylated polymer can be achieved (see, PCT Patent Publication No. WO 1997/021452) and (2) polymers with varying number of carboxyl groups can be synthesized by varying the amount of a halogenated acid (e.g., bromoacetic acid, chloroacetic acid, bromohexanoic acid, and chlorohexanoic acid) to achieve an optimum level of carboxylation as required for the synthesis of polymer coated nanoparticles (see, PCT Patent Publication No. WO 2000/061191). Alternatively, polycarboxylic acid functional polymers, such as polymethacrylic acid based polymers, can be synthesized directly.

Naturally-occurring hydroxylated polymers that can be used in the synthesis of polycarboxylated polymers include polysaccharides like dextran, starch, or cellulose. Polyvinyl alcohol is a synthetic hydroxylated polymer that can replace naturally-occurring polysaccharides. These hydroxyl group-bearing polymers can be reacted with halogenated acids in the presence of a strong base, typically 1-8 M NaOH. The polycarboxylated polymer can then be purified by ultrafiltration or by precipitation. Alternatively, anhydrides, like succinic anhydride, can be used for carboxylation of polyhydroxylated polymers. It is important to note, however, that the resulting ester linkages can undergo slow hydrolysis.

Reaction of positively charged polymers like polylysine or polyvinyl amine with an anhydride (e.g., succinic anhydride, maleic anhydride, DTPA anhydride) is another approach for synthesizing carboxylated polymers, as is hydrolysis of an anhydride-containing polymer, such as polyethylene-g-maleic anhydride (Sigma-Aldrich, St. Louis, Mo.).

Carboxyl group-bearing polyamino acids can also be employed as polycarboxylated polymers (e.g., polyaspartate or polyglutamate). Carboxylated dendrimers, which are available commercially, are highly branched synthetic polymers that can be used as coatings to encapsulate nanoparticle cores.

Nanoparticle cores having carboxy groups can, for example, be synthesized by mixing a carboxy terminated polymer with ferrous and ferric salts. Metals other than iron (e.g., zinc, manganese, or cobalt) can be used in the synthesis of magnetic metal oxides and can partially or completely replace the ferrous ion during the synthesis of magnetic metal oxides.

Carboxylation reactions can be performed in a jacked reactor for temperature control, stirred, and covered to control access of oxygen. The reaction mixture can then be brought to controlled temperature between 4° C. and 20° C., and a base, such as ammonia, can be added drop-wise or with a pump. Sufficient base is added to bring the pH to higher than pH 8, which causes the formation of iron oxides. The resulting gel or colloid is then heated to induce the formation of the highly magnetic iron oxide. After 30 minutes at 60° C., the colloid is cooled and unreacted polymer is removed from the polymer coated nanoparticle, such as by ultrafiltration with a membrane that has a cutoff that permits the carboxylated polymer to pass through while retaining the larger coated nanoparticle. Alternatives to ultrafiltration include gel filtration and magnetic separation. Citrate may be added as stabilizer but it must be removed by ultrafiltration before use of carbodiimide because of its carboxyl groups.

U.S. Patent Publication No. 2007/0258907 describes the coating of paramagnetic and superparamagnetic nanoparticle cores with a cyclodextrin (CD)-containing polymeric compound wherein the cyclodextrin-containing polymer comprises from 1 monomeric unit to 30,000 monomeric units.

Nanoparticle cores can be functionalized with carboxyl groups by employing carboxyl-bearing polymers such as, for example, the carboxymethyl (‘CM’) polysaccharides CM-cellulose, CM-dextran, and CM-arabinogalactan, which can be produced in a reaction of polysaccharide and haloacetic acid. See, U.S. Pat. No. 5,981,507.

Carboxylated dextrans can also be used to encapsulate nanoparticle cores, including superparamagnetic iron oxide nanoparticle cores. See, Hasegawa, U.S. Pat. Nos. 4,101,435 and 5,424,419. Carboxydextrans have a single terminal carboxyl group on each dextran molecule. Carboxymethylated dextrans have numerous carboxymethyl groups attached per mole of dextran and may be prepared in a reaction of alkyl halogenated acids in base as described in Maruno, U.S. Pat. No. 5,204,457 and Groman, PCT Patent Publication No. WO 2000/061191.

Nanoparticle cores can also be encapsulated with dextran that is cross-linked with epichlorohydrin by reacting epoxy groups with ammonia to generate amine groups. Iron oxide nanoparticle cores encapsulated with cross-linked dextran are known in the art and referred to as cross-linked iron oxide or “CLIO” as discussed in further detail herein. When functionalized with an amine group CLIO are referred to as amine-CLIO or NH₂—CLIO.

A dextran-coated nanoparticle core can be formed and then treated with periodate to produce aldehyde groups, which react with amino groups to form a Schiff base that may be stabilized by treatment with a reducing agent, like sodium borohydride. Such dextran-coated nanoparticle cores are suitable for use with a methylene amino linker. See, discussion of cross-linking agents elsewhere herein.

Carboxyl groups on carboxyl-terminated nanoparticle cores can be activated with a water soluble carbodiimide in, for example, a non-amine containing buffer of from pH 4.5 to pH 7 at a temperature of from 20° C. to 40° C. Activation with 0.1 M TEMED may be achieved at pH 4.8. A variety of diamines, such as hexamine diamine, ethylene diamine, spermidine, spermine and/or as well as the amino acids ornithine and/or lysine, can be added to the activation reaction to block crosslink formation between the carboxyl groups. Excess diamine can be separated from the aminated nanoparticle core using ultrafiltration. Carbodiimide results in the formation of a peptide bond between the diamine linker and nanoparticle core coating, such as a polymer coating. The number of primary amines on the nanoparticle core can be controlled by reaction with trinitrobenze.

Amino-functionalized nanoparticle cores can be used for the attachment of biologically active molecules and/or cell membrane-penetrating molecules either directly or through a bifunctional crosslinking having a first and second functional group, such as the bifunctional crosslinking agents discussed herein or as otherwise available in the art. Suitable functional groups for reacting with amino-functionalized nanoparticles include NHS esters, which react with the amine group of a nanoparticle and have a second functional group that can react with a sulfhydryl group of a bioactive molecule or a cell membrane-permeating molecule. Such crosslinking agents include, for example, SPDP, long chain-SPDP, SIA, MBS, SMCC, and others that are well known in the art and are commercially available (e.g., Piece Chemical Company, Rockford, Ill.).

Gold nanoparticle cores may also be coated with polyethylene glycol (PEG) or with lipids to enhance biocompatibility and reduce toxicity. A PEG spacer may be used to improve the efficiency of gold nanoparticle attachment to molecules, including biologically active molecules and cell penetrating molecules, to increase the accessibility and activity of molecules as compared to molecules that are directly attached to a nanoparticle without a spacer.

Amino-functionalized nanoparticle cores can also be synthesized using non-crosslinked, carboxylated polymers, including natural polymers, synthetic polymers, or derivatives of each such as, for example, polyvinyl alcohol and carboxymethyl dextran (CM), which permits the addition of reactive primary amine groups to the polymer through peptidyl linkages. Such noncrosslinked carboxylated polymer coated nanoparticles have two classes of carboxyl groups with distinct chemical reactivities. Some carboxyl groups are shielded from further chemical reaction by forming a strong bond between the polymer and the surface of the iron oxide while other carboxyl groups face the bulk solvent and can be converted to reactive primary amino groups with carbodiimide.

Amino groups can be associated with polymer through a peptidyl linkage of the formula: —O—(CH₂)ₘ—CONH—[X], wherein X is —(CH₂)ₙNH₂, —(CH₂)ₒCH NHCOO—, —(CH₂)₃NH(CH₂)₄NH₂, or —(CH₂)₃NH(CH₂)₄NH(CH₂)₃NH₂—, wherein m=1, 2, or 3; n=2, 3, 6, and o=3 or 4.

Such amine-functionalized nanoparticle cores can be readily degraded to yield metal salts and the residual polymer coating. In vivo, this results in the utilization of iron oxide, by incorporation of iron into red blood cells, and by the excretion and/or degradation of the polycarboxylated polymer. In vitro, the conditions of biodegradation can be simulated by exposing nanoparticles to mildly acidic pH (3-6) in the presence of a metal chelator, e.g. citrate or EDTA. This yields ferric ion chelates and soluble polyfunctional polymers. The molecular weight of the polyfunctional polymers, now bearing amino and carboxyl groups, will be slightly larger than the polycarboxylated polymers used to synthesize the nanoparticles.

A dextran shell surrounding an iron oxide core can stabilize a nanoparticle thereby permitting the storage of functionalized nanoparticles under a wide range of temperatures, pH, and/or ionic strengths, either in an unconjugated form or as functionalized nanoparticles having one or more biologically active molecules and/or one or more cell membrane-penetrating molecules.

Dextran and other materials may be added to make nanoparticle cores biofriendly. This includes coating the nanoparticle with polyethylene glycol (Peg) or adding lipids to the nanoparticle. Exemplary syntheses can be found in the literature, such as Synthesis, Surface Modification and Characterization of Nanoparticles. L. S. Wang and R. Y. Hong. (2011) in Advances in Nanocomposites Synthesis, Characterization and Industrial Applications, Dr. Boreddy Reddy, editor. Intech China publisher, Superparamagnetic iron oxide nanoparticles functionalized with peptides by electrostatic interactions. Hildebrandt et al., Arkivoc 79 (2007). Chemically prepared magnetic nanoparticles. Willard et al., International Materials Reviews 49:125-170 (2004) (further described elsewhere herein).

The outer surface of the nanoparticle cores and/or coatings encapuslating nanoparticle cores can be modified by mixing the nanoparticles with adamantane-PEG (AD-PEG) at a 1:1 molar ratio of cyclodextrin to AD-PEG. Adamantane interacts with the polymer by forming a stable inclusion complex with cyclodextrin. PEG is exposed to the solvent, which stabilizes the nanoparticles under physiological conditions.

The outer surface of the nanoparticles can be further modified by the attachment of a ligand to adamantane-PEG. For example, biologically active molecules and/or targeting molecules are mixed with a nanoparticle core solution at 1.7% w/w of AD-PEG-molecule to AD-PEG. Molecules are covalently attached to AD-PEG. When an AD-PEG-molecule is mixed with the nanoparticle cores, the molecule is displayed on the outside of the nanoparticle core. In their final form, the nanoparticle cores have PEG and one or more molecule(s) displayed on the outside of the complex.

Avidin or streptavidin can be attached to nanoparticle cores for use in conjunction with a biotinylated binding moiety, such as an oligonucleotide or polypeptide, a biotinylated cell membrane-penetrating molecule, and/or a crosslinking agent. See, for example, Shen et al., Bioconjug. Chem. 7(3):311-6 (1996). Similarly, biotin can be attached to nanoparticles for use with an avidin-labeled biologically active molecule, an avidin-labeled cell membrane-penetrating molecule, and/or an avidin-labeled crosslinking agent.

A non-polymeric coating of DMSA can be formed on a surface of a synthetic nanoparticle shell via the methodology of Albrecht et al., Biochimie 80(5-6):379-90 (1998). DMSA can be coupled to a synthetic ferrite shell thereby providing an exposed functional group.

Dextran-coated nanoparticle cores can be made and cross-linked with epichlorohydrin. The addition of ammonia will react with epoxy groups to generate amine groups as is described, for example, in U.S. Patent Publication Nos. 2003/0124194 and 2003/0092029 and by Hogemann et al., Bioconjug. Chem. 11(6):941-6 (2000) and Josephson et al., Bioconjug. Chem. 10(2):186-91 (1999). This material is known as cross-linked iron oxide or “CLIO” and when functionalized with amine is referred to as amine-CLIO or NH₂-CLIO.

A second property of amine functionalized polymers of the present disclosure is the presence of at least two nitrogen atoms for each primary amine due to characteristic general structure (H₂N—X—NH—CO—). X can be any structure connecting the two primary amines of the diamine. Non-limiting examples of X include hexamine diamine, ethylene diamine, spermidine or spermine, and amino acids like ornithine or lysine, which are of interest due to their negatively charged carboxyl group. The total number of nitrogen groups attached to the purified polymer can be obtained by submitting the purified polymer to elemental analysis of nitrogen (i.e., determination of the content of all nitrogen atoms).

The number of reactive primary amino groups can be determined by the TNBS method. A property of certain amine-functionalized polymers of the present disclosure is that the amount of total nitrogen may exceed the amount of nitrogen present as a primary amine. For example, when ethylene diamine (EDA) is used, the total nitrogen content will be twice the nitrogen content obtained with methods determining the amount of primary amine.

Polycarboxylated polymers may be obtained by a variety of routes and have a variety of compositions. They may be man-made or naturally occurring and may be highly branched or linear. The polycarboxylated polymers have a molecular weight between about 5 and 200 kDa, more preferably between 5 and 50 kDa. Smaller polymers lack sufficient carboxyl groups to both strongly bind the iron oxide and to have the requisite free carboxyl groups available for conversion to amino groups. The polymers must contain more than about five moles of carboxyl group per mole of polymer. The number of carboxyl groups can be determined by titration. The polycarboxylated polymers should have a high water solubility over a wide pH range to be employed in the synthesis of water soluble polymer coated functionalized nanoparticles.

For in vivo uses, the molecule-nanoparticle conjugates are formulated and sterilized according to published methods for sterilizing parenterally-administered MRI contrast agents. For parenteral applications, sterilization can be achieved by filtering the colloid through a 220 nm filter (filter sterilization) or by heat sterilization (terminal sterilization). Depending on the method of sterilization, various excipients, such as monosaccharides, polysaccharides, salts, can be added to stabilize the colloid during heat stress or storage. Excipients can also serve to bring the ionic strength and pH of the preparation into the physiological range. (See, Josephson, U.S. Pat. No. 5,160,726 and Groman, U.S. Pat. No. 5,248,492).

Carboxymethylated polymers can be prepared by reaction of a halo acetic acid with a polymer in strong base, usually NaOH. The polymer should be of sufficient molecular weight to allow separation of unreacted haloacetic acid from the carboxymethylated polymer. The polymer is preferably between 5 kDa and 100 kDa. The separation can be accomplished by dialysis, ultrafiltration or precipitation. The polymer is then dried by lyophilization, vacuum drying or spray drying. The polymer should be of sufficient molecular weight to allow separation of dextran from dextran-coated iron oxide. For example, separation can be accomplished by ultrafiltratoin when the nanoparticles have molecular weights of greater than 500 kDa, and the polymer is preferably less than 100 kDa.

Carboxymethylated polymer-coated nanoparticles can be prepared in a solution of 12 mmoles of ferric chloride (hexahydrate) and 6 grams of CM-PVA. 6 mmoles of ferrous chloride (tetrahydrate) can then be added with stirring followed by the dropwise addition of 28-30% ammonium hydroxide (2-4° C.). The mixture can then be heated to between 70 and 90° C. and maintained at the higher temperature for 2 hours. Unreacted CM-PVA was removed by ultrafiltration using a 100 kDa cutoff membrane. The colloid had a size of 54 nm by light scattering and an R2 of 60 mM-1 sec-1. The procedure was repeated using 3 g CM-PVA to give a colloid with 65 nm and an R2 of 160 mM-1 sec-1.

Carboxyl groups on the carboxylated polymer coated nanoparticles can be converted to amino groups in 0.1 M TEMED buffer, pH 4.8, was added 0.2 g of 1-ethyl-3-(dimethylaminopropyl) carbodiimide hydrochloride at room temperature. After 15 minutes, 0.5 mL 1,2 ethylene diamine was added. After 24 hours the mixture was put in dialysis bag and dialyzed until the dialysate was free of amine by the TNBS assay.

The biologically active molecule or targeting molecule, such as a cell membrane-penetrating molecule, preferably with a single sulfhydryl group distal from the site of bioactivity, is allowed to react with the activated nanoparticle. Separation of unreacted biomolecule from the biomolecule-nanoparticle conjugates can be accomplished by gel filtration, ultrafiltration, dialysis or magnetic separation methods. Examples of thiolated biomolecules that have been attached to SPDP-activated crosslinked magnetic nanoparticles include transferrin, (Hogemann, Bioconjug Chem 11:941-6 (2000)), tat peptides (Josephson, Bioconjug Chem 10:186-91 (1999) and Zhao Bioconjug Chem 13:840-4 (2002)), oligonucleotides (Josephson, Agnew Chem Int Ed 40:3204-3206 (2001) and Perez, J Am Chem Soc 12:2856-7 (2002)), antibodies (Kang, Bioconjug Chem 13:122-7 (2002)) and proteins (Perez, Nature Biotechnol 20:816-20 (2002)). For peptides (1-2 kDa), 5-25 peptides can be attached per 2000 Fe atoms. For proteins, such as transferrin or antibodies (50-200 kDa) 1-4 biomolecules can be attached per 2000 Fe atoms.

A4. Crosslinking Agents

Within certain embodiments, the functionalized nanoparticles that are disclosed herein include one or more crosslinking agents, most commonly bifunctional crosslinking agents, to attach one or more biologically active molecule(s) and/or one or more targeting molecules, including cell membrane-penetrating molecule(s), or other targeting molecule, to a nanoparticle core and/or to a coating that encapsulates a nanoparticle core.

As used herein the terms “crosslinking agent” and “linker” are used interchangeably and refer to any straight chain or branched, symmetric or asymmetric compound. Crosslinking agents may include two or more functional groups through which reaction and thus linkage biologically active molecules and/or cell targeting molecules can be achieved. Examples of functional groups, which may be the same or different, terminal or internal, of each linker group include, but are not limited, to amino, acid, imidazole, hydroxyl, thio, acyl halide, —C═C—, or —C≡C— groups and derivatives thereof. In preferred embodiments, the two functional groups are the same and are located at termini of the comonomer. In certain embodiments, a linker group contains one or more pendant groups with at least one functional group through which reaction and thus linkage of therapeutic agent or targeting ligand can be achieved, or branched polymerization can be achieved. Examples of functional groups, which may be the same or different, terminal or internal, of each linker group pendant group include, but are not limited, to amino, acid, imidazole, hydroxyl, thiol, acyl halide, ethylene, and ethyne groups and derivatives thereof. In certain embodiments, the pendant group is a (un)substituted branched, cyclic or straight chain C1-C10 (preferably C1-C6) alkyl, or arylalkyl optionally containing one or more heteroatoms, e.g., N, O, S, within the chain or ring.

As used herein, the terms “cross-linking agent” or “linker” include long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pypridyldithio)-proprionate (SPDP); long-chain N-Succinimidyl-3-(pypridyldithio)-proprionate (LC-SPDP); sulfo-N-Succinimidyl-3-(pypridyldithio)-proprionate (sulfo-SPDP); long-chain sulfo-N-Succinimidyl-3-(pypridyldithio)-proprionate (sulfo-LC-SPDP); 1-Ethyl Hydrocholride-3-(3-Dimethylaminopropyl) carbodiimide (EDC); long-chain 1-Ethyl Hydrocholride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pypridyldithio)-proprionate polyethylene glycolₘ (SPDP(PEG)ₘ); and sulfo-N-Succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

In an exemplary reaction, succinimidyl 4-(N-maleimidomethyl) cyclohexane-1-carboxylate (SMCC; Thermo Fisher 22360) is dissolved at a concentration of 1 mg/ml in dimethylformamide (DMF; Thermo Fisher 20673) and added, in a large excess of SMCC over available amine groups, to a suspension of amino-SPION. The reaction is allowed to proceed for approximately one hour. It should also be noted that SMCC also can be purchased as a sulfo derivative (Sulfo-SMCC), making it more water soluble. DMSO may also be substituted for DMF as the solvent carrier for the labeling reagent; again, it should be anhydrous. Excess SMCC and DMF can be removed using an Amicon centrifugal filter column with a cutoff of 3,000 daltons. Five exchanges of volume are generally required to ensure proper buffer exchange and complete removal of excess SMCC.

Suitable functional groups for reacting with amino-functionalized nanoparticles include NHS esters, which react with the amine group of a nanoparticle and have a second functional group that can react with a sulfhydryl group of a bioactive molecule or a cell membrane-permeating molecule. Such crosslinking agents include, for example, SPDP, long chain-SPDP, SIA, MBS, SMCC, and others that are well known in the art and are commercially available, for example, from Piece Chemical Company (Rockford, Ill.).

Other crosslinking agents that may be employed in the presently-disclosed functionalized nanoparticles include succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-succinimidyl-3-(pypridyldithio)-proprionate polyethylene glycolₘ (SPDP(PEG)ₘ); and sulfo-N-succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units. PEG can include one or more carboxyl groups and one or more amine groups; one or more carboxyl groups and one or more sulfhydryl groups; two or more carboxyl groups; and/or two or more sulfhydryl groups.

Further crosslinking agents that may be employed in the presently-disclosed functionalized nanoparticles include N-succinimidyl-3-(pypridyldithio)-proprionate (SPDP); long-chain N-succinimidyl-3-(pypridyldithio)-proprionate (LC-SPDP); sulfo-N-succinimidyl-3-(pypridyldithio)-proprionate (sulfo-SPDP); and long-chain sulfo-N-succinimidyl-3-(pypridyldithio)-proprionate (sulfo-LC-SPDP). Representative long-chain variants of SPDP include, for example, LC1-SPDP and LC2-SPDP, as well as N-hydroxysuccinimide(NHS)-LC-SPDPs, such as NHS-LC-SPDP and NHS-LC2-SPDP and sulfo-LC-SPDPs, such as sulfo-LC1-SPDP and sulfo-LC2-SPDP.

SMCC, sulfo-SMCC, NHS-SMCC, LC-SMCC, sulfo-LC-SMCC, NHS-LC-SMCC, SPDP, sulfo-SPDP, NHS-SPDP, LC-SPDP, sulfo-LC-SPDP, and NHS-LC-SPDP can be employed as crosslinking agents for nanoparticle core surfaces, polymer coatings, biologically active molecules, and/or cell targeting molecules that include one or more amino groups and one or more thiol groups.

Additional crosslinking agents that may be employed in the presently-disclosed functionalized nanoparticles include 1-ethyl hydrochloride-3-(3-dimethylaminopropyl) carbodiimide (EDC) and long-chain variants of 1-ethyl hydrochloride-3-(3-dimethylaminopropyl) carbodiimide (LC-EDC). Representative long-chain variants of EDC include, for example, LC1-EDC and LC2-EDC, as well as N-hydroxysuccinimide(NHS)-LC-EDCs, such as NHS-LC1-EDC and NHS-LC2-EDC and sulfo-LC-EDCs, such as sulfo-LC1-EDC and sulfo-LC2-EDC.

Crosslinking agents known in the art as AMAS, BMPS, GMBS, MBS, SMPB, SMPH, LC-SMCC, KMUS, Imodiester crosslinker dimethyl suberimidate, BS3, Formaldehyde, and EDC may also be used to prepare the functionalized nanoparticles disclosed herein. EDC and LC-EDC can, for example, be employed as crosslinking agents for nanoparticle core surfaces, polymer coatings, biologically active molecules, and/or cell targeting molecules that include one or more —COOH groups and one or more —NH₂ groups.

An activated biologically active molecule and/or cell targeting molecule, preferably with a single sulfhydryl group distal from the site of bioactivity, is allowed to react with the activated nanoparticle. Separation of unreacted molecules from the molecule-nanoparticle conjugates can be accomplished by gel filtration, ultrafiltration, dialysis or magnetic separation methods. For peptides (1-2 kDa), 5-25 peptides can be attached per 2000 Fe atoms. For proteins, such as transferrin or antibodies (50-200 kDa) 1-4 biomolecules can be attached per 2000 Fe atoms.

For further detail on the attachment of thiolated biomolecules to SPDP activated crosslinked nanoparticle cores see, Hogemann, Bioconjug Chem 11:941-6 (2000) (transferrin), Josephson, Bioconjug Chem 10:186-91 (1999) and Zhao Bioconjug Chem 13:840-4 (2002) (tat peptides), Josephson Agnew Chem Int Ed 40:3204-3206 (2001) and Perez, J Am Chem Soc 124:2856-7 (2002) (oligonucleotides), Kang, Bioconjug Chem 13:122-7 (2002) (antibodies), and Perez, Nature Biotechnol 20:816-20 (other proteins).

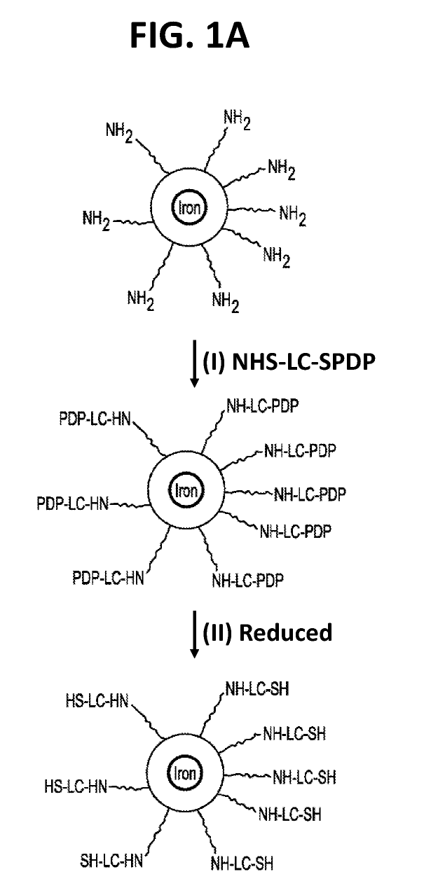

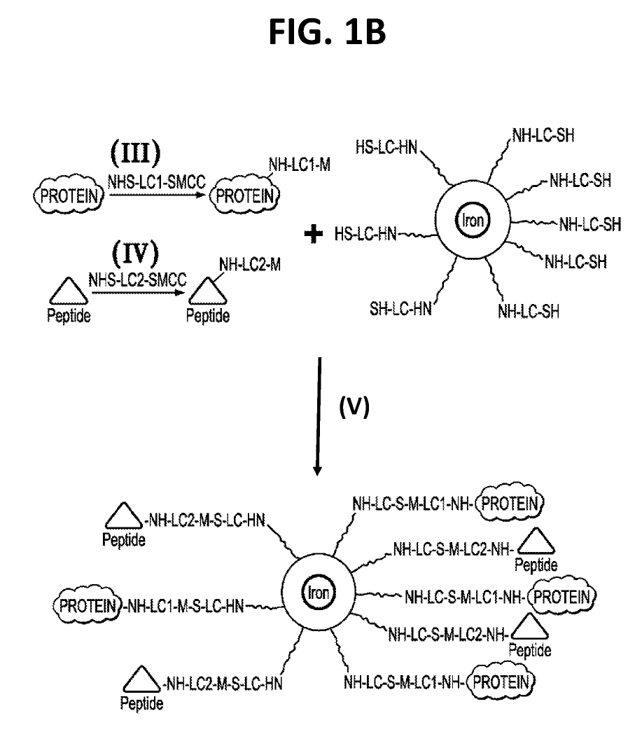

FIG. 1 depicts schematic representation of nanoparticle functionalization and binding of targeting molecules or biologically active molecules to a nanoparticle core. NHS-LC-SPDP (Thermo Fisher) is a long chain cross-linking agent (extender) with bifunctional coupling reagents on either side; the bicoupling reagents are specific for amines, permitting conversion of a disulfide bond to a sulfide.

One end has an N-Hydroxysuccinimide ester, while the other end of the extender contains a pyridyldithiol group. This dithiol group can be reduced to produce a sulfuydryl. NHS-LC-SPDP is allowed to react with the nanoparticles and the reaction can be cleared from unincorporated NHS-LC-SPD. The coupled nanoparticles are then reduced as shown in FIG. 1.

The biologically active proteins purified using affinity columns contain a free epsilon-amine group from carboxy-terminal lysine residue, which is added to facilitate binding to the nanoparticles. NHS-LC-SMCC is used as the bifunctional coupling reagent. The molecule has an LCI chain extender. One end has the N-Hydroxysuccinimide reagent specific for amines. The other end contains the maleimide group, very specific for sulfuydryl groups. Once the material is coupled to a protein and separated from the reaction mixture, the maleimide coupled protein will be added to the sulfhydryl-containing nanoparticle. The resultant material is separated by gel filtration.

All the other crosslinking reagents can be applied in a similar fashion. SPDP is applied to the protein/applicable peptide in the same manner as SMCC and is readily soluble in DMF. As described previously, dithiols are severed by a reaction with DTT for an hour or more. After removal of byproducts and unreacted material, purification is performed by use of an Amicon centrifugal filter column with 3,000 MW cutoff.

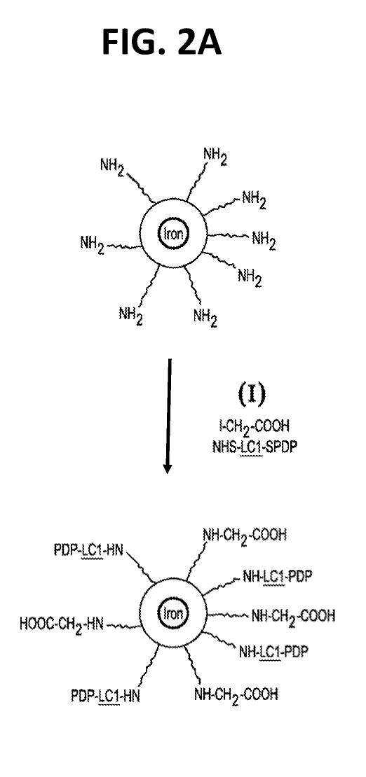

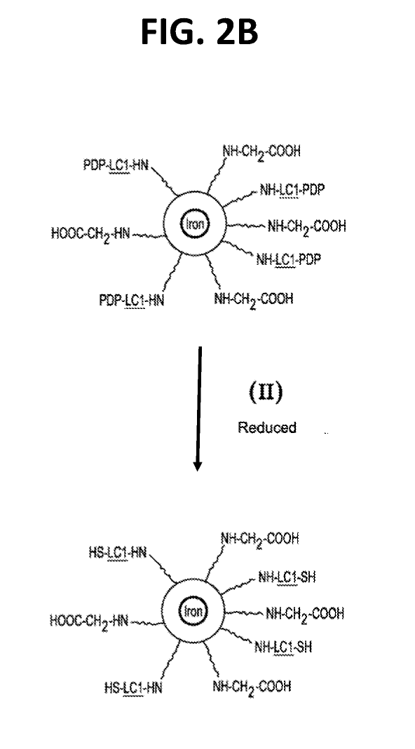

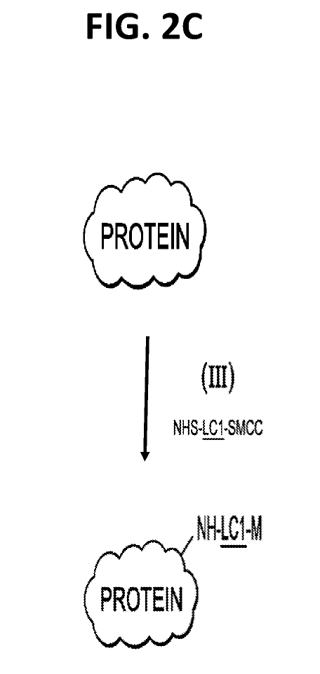

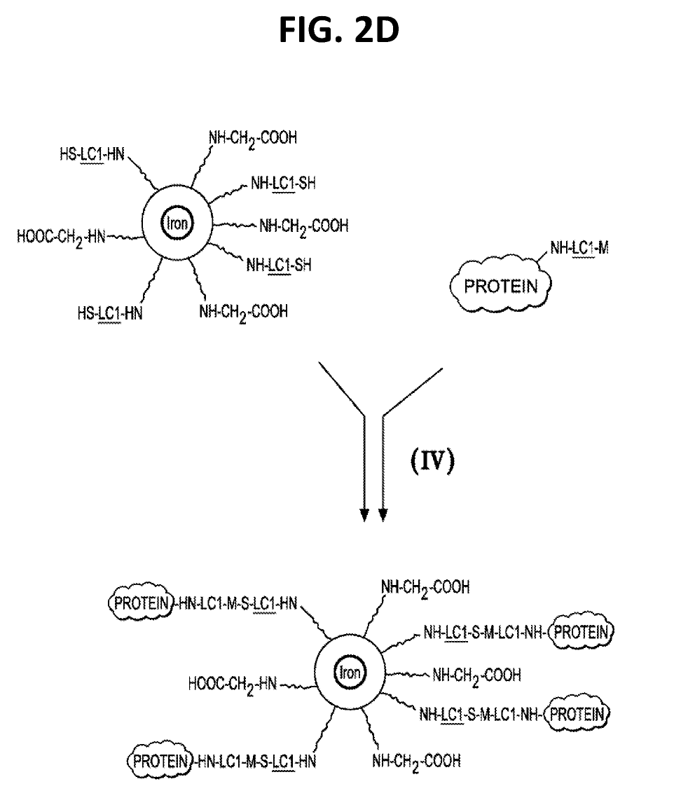

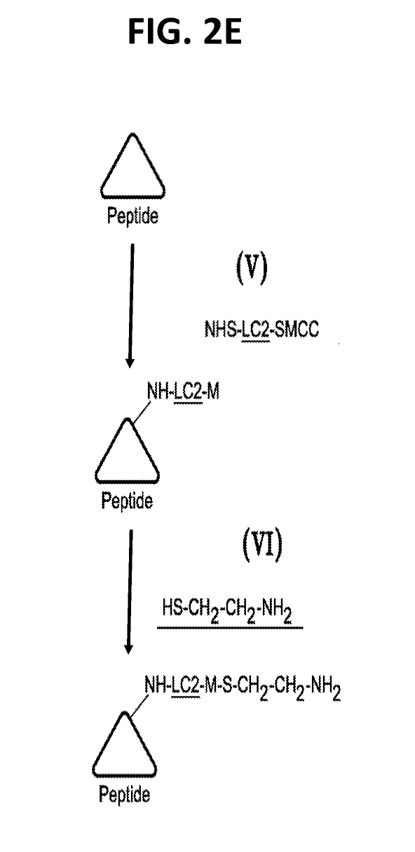

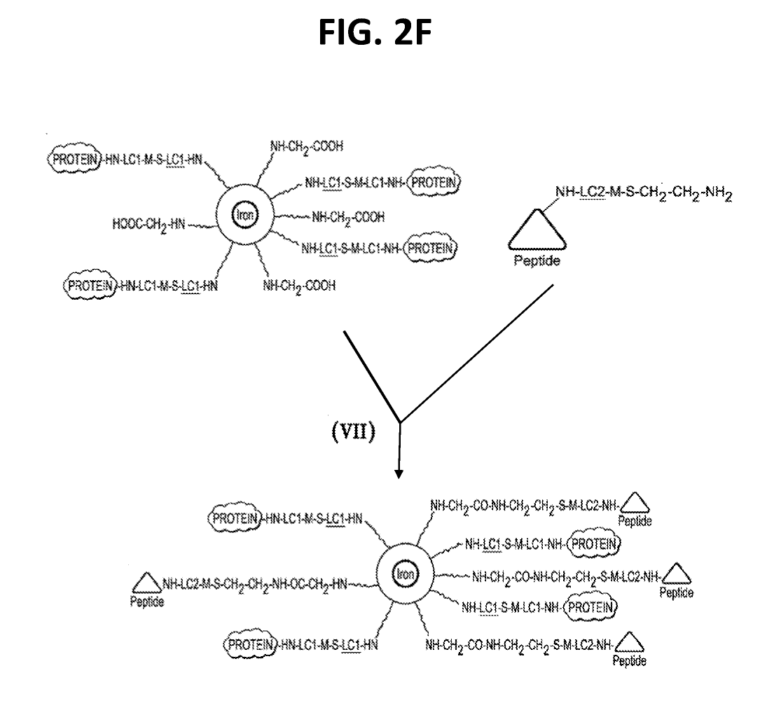

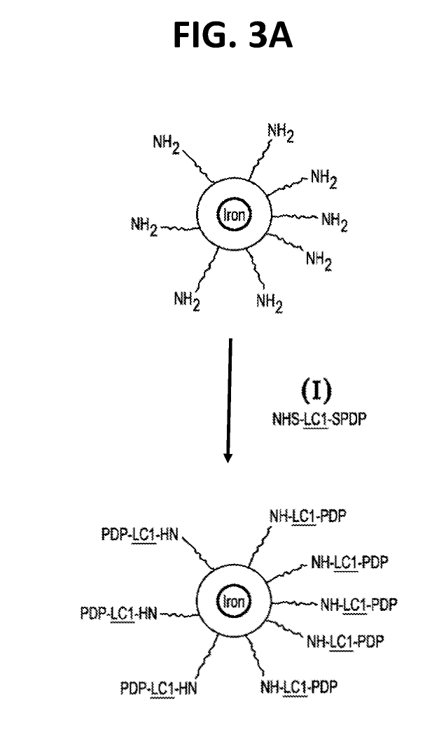

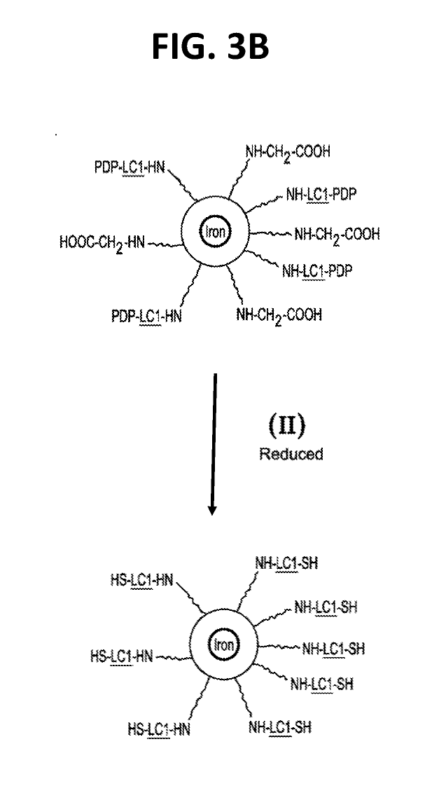

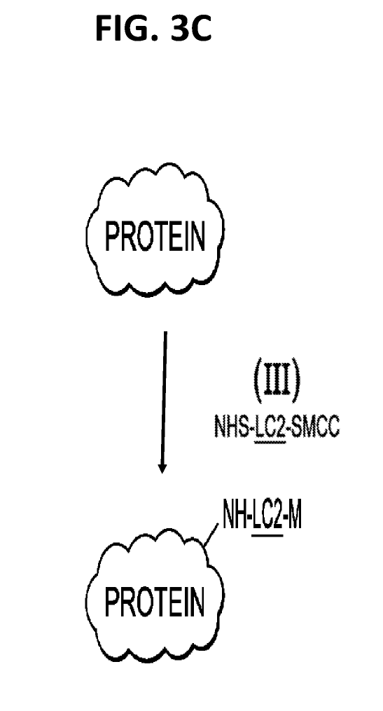

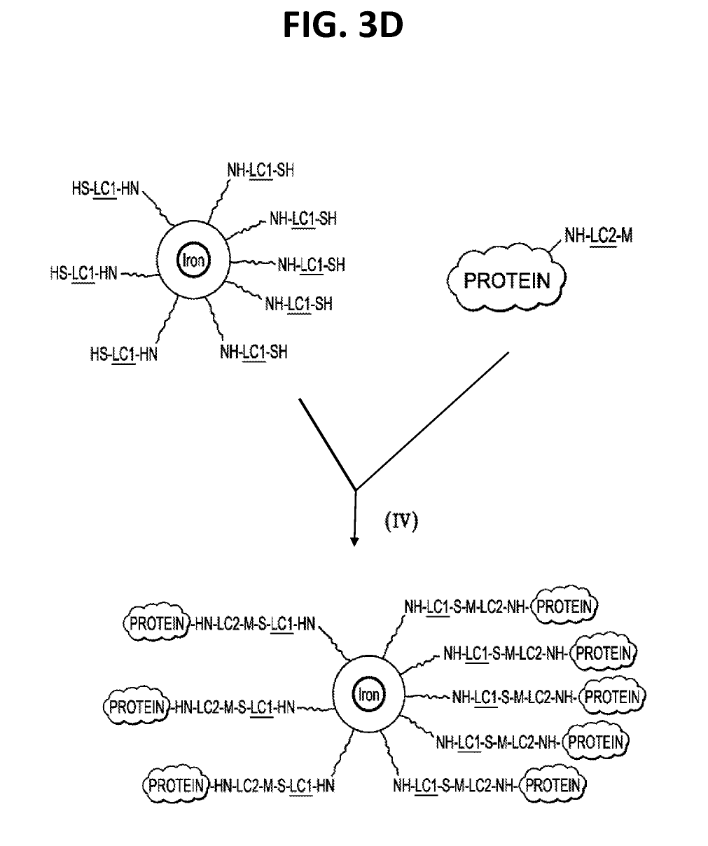

As shown in FIGS. 2A and 2B, an amino-SPION can be labeled with a peptide, polypeptide, and/or protein in a more direct and controlled means by using two different bifunctional coupling reagents, e.g., Iodoacetic acid (I—CH₂—COOH) and an N-Succinimidyl-3-(pypridyldithio)-proprionate (SPDP) linker (e.g., NHS-LC-SPDP), followed by a step of reduction to yield SPIONs having both carboxyl and sulfhydryl reactive groups.

The peptide attached to the LC-SMCC is treated with aminomercaptoethanol. This creates a linkage through the sulfhydryl group and provides a free amino group. This amino group is then coupled to the carboxyl group on the nanoparticle using EDC. EDC is known as 1-ethyl-3 [3-dimethylaminopropyl] carbodiimide hydrochloride. This coupling step is performed last in the reaction scheme.

In this case, the peptide also contains a carboxyterminal lysine that will serve as the base for the NHS ester-LC-maleimide coupling. The molecule has an LC2 chain extender. All procedures are similar to those describe above for the protein.

A5. Cell Membrane-Penetrating Molecules and Other Targeting Molecules

Within certain embodiments, functionalized nanoparticles according to the present disclosure include one or more targeting molecules for directing the functionalized nanoparticle to a specific tissue, cell, and/or subcellular compartment/organelle. Common targeting molecules include monoclonal antibodies, aptamers, streptavidin, and peptides.

Within various aspects of these embodiments, targeting molecules may be attached (1) directly to a nanoparticle core through the interaction of a functional group on the targeting molecule and a functional group on the nanoparticle core, (2) directly to a coating that encapsulates a nanoparticle core through the interaction of a functional group on the targeting molecule and a functional group on the coating, (3) indirectly to a nanoparticle core via a cross-linking molecule through (a) the interaction of a first functional group on the cross-linking molecule and a functional group on the nanoparticle core and (b) the interaction of a second functional group on the cross-linking molecule and a functional group on the targeting molecule, (4) indirectly to a coating that encapsulates a nanoparticle core via a cross-linking molecule through (a) the interaction of a first functional group on the cross-linking molecule and a functional group on the coating and (b) the interaction of a second functional group on the cross-linking molecule and a functional group on the targeting molecule.

Functionalized nanoparticles having multiple targeting molecules or target binding sites may be used advantageously to cluster receptors thereby activating cellular signaling pathways and increasing binding affinity and/or enhancing anchoring. Functionalized nanoparticles having a single binding site (i.e., monovalent functionalized nanoparticles) may be used advantageously to avoid clustering and may find use in applications that include the tracking of individual proteins is.

Within certain aspects of these embodiments, targeting molecules includes cell membrane-penetrating molecules that can bind to and penetrate through a mammalian cell membrane, such as a plasma membrane, a nuclear membrane, a mitochondrion membrane, and/or a membrane of another organelle, thereby facilitating the introduction of the functionalized nanoparticle into a target cell and delivery of one or more biologically active molecules that are attached directly or indirectly to nanoparticle core or coating that encapsulates a nanoparticle core of the functionalized nanoparticle.

Cell membrane-penetrating molecules include full-length proteins, polypeptides, and/or peptides; nucleic acids, such as cDNAs, RNAs, oligonucleotides, primers, and/or probes; and/or small molecules to facilitate (i) the cellular uptake of the functionalized nanoparticle via a mammalian cell plasma membrane and, optionally, (ii) the subcellular localization of the functionalized nanoparticle into a mammalian cell nucleus, mitochondria, lysosome, endosome, or other organelle via a mammalian cell nuclear membrane, mitochondrial membrane, lysosomal membrane, endosomal membrane, and/or other organelle membrane.

Within certain aspects, cell membrane-penetrating molecules may be cell penetrating peptides (CPPs), which facilitate the translocation of a functionalized nanoparticle through a plasma membrane of a target cell through one or more of direct penetration into the membrane, endocytosis-mediated entry, and/or via a transitory structure thereby affecting the delivery of various molecular cargoes to the cytoplasm, nucleus, or other organelle.

Cell penetrating peptides that may be used to prepare the functionalized nanoparticles disclosed herein include cell penetrating peptides having amino acid sequences derived from the trans-activating transcriptional activator (Tat) protein from Human Immunodeficiency Virus 1 (HIV-1). See, e.g., Terwogt et al., Cancer Treat. Rev. 23:87-95 (1997); Rait et al., Bioconjugate Chem. 11:153-160 (2000); Anderson et al., Biochem Biophys Res. Commun. 194:876-884 (1993); and Fawell et al., Proc. Natl. Acad. Sci. U.S.A. 91:664-668 (1994). Tat proteins are often characterized by comprising the amino sequence RKKRRQRRR, which corresponds with Tat amino acids 49-57. Cell penetrating peptides that may be used to prepare the functionalized nanoparticles disclosed herein may be cationic (positively charged), for example with several R (arginine) residues. Six or more R residues is known to work. Another positively charged amino acid is K (lysine) which is also present in different CPPs.

Exemplary cell penetrating peptides include HIV Tat, SynB1, SynB1, SynB3, PTD-4, PTD-4, PTD-5, SBP, MAP, Pep-1, and Pep-2. Other suitable cell penetrating peptides include HIV Tat-derived peptides and other peptides having, for example, from five to nine basic amino acids, including arginine and/or lysine.

Cell penetrating peptides that can be used to prepare the functionalized nanoparticles disclosed herein can have an amino acid composition that either contains a high relative abundance of positively-charged amino acids such as lysine or arginine or has sequences that contain an alternating pattern of polar/charged amino acids and non-polar, hydrophobic amino acids. These two structures are referred to as polycationic or amphipathic, respectively. A third class of CPPs are hydrophobic peptides, containing only apolar residues, with low net charge or which hydrophobic amino acid groups that are crucial for cellular uptake.

Without being bound by theory, it is believed that, depending upon the structure of the specific cell membrane-penetrating molecule used, functionalized nanoparticles disclosed herein may translocate directly across a plasma membrane, including via an interaction between a cell membrane-penetrating molecule and phosphate groups on both sides of the lipid bilayer, the insertion of charged side-chains that nucleate the formation of a transient pore, followed by the translocation of cell-penetrating peptides by diffusing on the pore surface. This mechanism explains how key ingredients, such as the cooperativity among the peptides, the large positive charge, and specifically the guanidinium groups, contribute to the uptake.

The proposed mechanism also illustrates the importance of membrane fluctuations. Indeed, mechanisms that involve large fluctuations of the membrane structure, such as transient pores and the insertion of charged amino acid side-chains, may be common and perhaps central to the functions of many membrane protein functions. This model contains several controversial features, may be the most striking one is the formation of transient pores that facilitate the diffusion of the peptides across either the plasma membrane or the endosomal vesicles towards the cytosol. Recent experimental data has validated this key ingredient of the model showing that cell-penetrating peptides indeed form transient pores on lipid bilayers and on live cells.

Endocytosis is the second mechanism liable for cellular internalization. Endocytosis is the process of cellular ingestion by which the plasma membrane folds inward to bring substances into the cell. During this process cells absorb material from the outside of the cell by imbibing it with their cell membrane. The classification of cellular localization using fluorescence or by endocytosis inhibitors is the basis of most examination. However, the procedure used during preparation of these samples creates questionable information regarding endocytosis. Moreover, studies show that cellular entry of penetratin by endocytosis is an energy-dependent process. This process is initiated by polyarginines interacting with heperan sulphates that promote endocytosis. Research has shown that TAT is internalized through a form of endocytosis called macropinocytosis.

Studies have illustrated that endocytosis is involved in the internalization of CPPs, but it has been suggested that different mechanisms could transpire at the same time. This is established by the behavior reported for penetratin and transportan wherein both membrane translocation and endocytosis occur concurrently.

The third mechanism responsible for the translocation is based on the formation of the inverted micelles. Inverted micelles are aggregates of colloidal surfactants in which the polar groups are concentrated in the interior and the lipophilic groups extend outward into the solvent. According to this model, a penetratin dimer combines with the negatively charged phospholipids, thus generating the formation of an inverted micelle inside of the lipid bilayer. The structure of the inverted micelles permits the peptide to remain in a hydrophilic environment. Nonetheless, this mechanism is still a matter of discussion, because the distribution of the penetratin between the inner and outer membrane is non-symmetric. This non-symmetric distribution produces an electrical field that has been well established. Increasing the amount of peptide on the outer leaflets causes the electric field to reach a critical value that can generate an electroporation-like event.

The last mechanism implies that internalization occurs by peptides belong to the family of primary amphipathic peptides, MPG and Pep-1. Two very similar models have been proposed based on physicochemical studies, consisting of circular dichroism, Fourier transform infrared, and nuclear magnetic resonance spectroscopy. These models are associated with electrophysiological measurements and investigations that have the ability to mimic model membranes such as monolayer at the air-water interface. The structure giving rise to the pores is the major difference between the proposed MPG and Pep-1 model. In the MPG model, the pore is formed by a b-barrel structure, whereas the Pep-1 is associated with helices. In addition, strong hydrophobic phospholipid-peptide interactions have been discovered in both models. In the two peptide models, the folded parts of the carrier molecule correlate to the hydrophobic domain, although the rest of the molecule remains unstructured.

Nucleic acid-based macromolecules such as siRNA, antisense oligonucleotide, decoy DNA, and plasmid have been realized as promising biological and pharmacological therapeutics in regulation of gene expression. However, unlike other small-molecular drugs, their development and applications are limited by high molecular weight and negative charges, which results in poor uptake efficiency and low cellular traffic. To overcome these problems, several different delivery systems have been developed, including a cell membrane-penetrating protein-nucleic acid conjugate, which is a very powerful tool.

Most cell membrane-penetrating protein-nucleic acid complexes that have been proposed so far are formed through covalent bonding. A range of cell membrane-penetrating protein-nucleic acid complexes have been synthesized through different chemistries that are either stable or cleavable linkages. The most widely-used method in publication is cleavable disulfide linkages through total stepwise solid-phase synthesis or solution-phase or solid-phase fragment coupling.

An exemplary covalent-bonding complexing strategy involves short interfering RNA (siRNA), which can interfere with and silence the expression of specific disease gene and find use in certain of the functionalized nanoparticles disclosed herein for the treatment of diseases and disorders, including certain cancers, hematopoietic diseases, neurological diseases, and genetic disorders. To improve cellular uptake of siRNA, cell membrane-penetrating molecules as described in detail herein may be employed to facilitate the delivery of siRNA into cells through either covalent or non-covalent linkages.

siRNAs may be attached to nanoparticle cores, coatings, and crosslinking agents by disulfide-linkage at 5′-end of the sense strands of siRNA. Alternatively, siRNAs may be attached to nanoparticle cores, coatings, and crosslinking agents through a stable thiomaleimide linkage at 3′-end of siRNA.

Stable amide, thiazolidine, oxime and hydrazine linkages have also been described in the art. One skilled in the art will appreciate that those linkages may alter the biological activity of molecules attached via these linkages. Short amphipathic molecules, including cell membrane penetrating molecules such as MPG and Pep-1 may be attached to nanoparticle cores, coatings, and crosslinking agents via non-covalent electrostatic and/or hydrophobic interactions, which have minimal effect on the biological activity of the attached molecules.

Non-covalent strategies may also be employed for attachment of siRNA to nanoparticle cores, coatings, and crosslinking agents. For example, it is known in the art that MPG/siRNA complexes that are formed through stable non-covalent interactions may be employed to introduce siRNAs into mammalian cells for the regulation of a target mRNA. MPG forms highly stable complexes with siRNA with a low degradation rate and can be easily functionalized for specific targeting, which may be advantageous over covalent attachment of biologically active and cell targeting molecules, including cell membrane-penetrating molecules.

Other non-covalent attachments have been described for secondary amphipathic peptides that are based on aromatic tryptophan and arginine residues linked with lysine as a spacer (CADY). CADY contains a short peptide sequence of 20 amino acids, with the sequence “Ac-GLWRALWRLLRSLWRLLWRA-cysteamide.” This peptide self-assembles into a helical shape with hydrophilic and hydrophobic residues on different sides of the molecule with two different orientations that represent the lowest energy. CADY can form complexes with siRNA at different molar ratios varying from 1:1 to 80:1 and is effective in protecting siRNA molecules from in vivo biodegradative processes that may occur prior to cellular penetration. Peptide nucleic acid (PNA) and phosphorodiamidate morpholino oligomers (PMO or Morpholino) also may be used to protect siRNA from degradation and may be attached to cell targeting molecules through disulfide linkages or through stable amide bonds.

Decoy DNA is an exogenous double-strand DNA (dsDNA), which can mimic a promoter sequence that can inhibit the activity of a specific transcription factor. But dsDNA has the same problem as other therapeutics, poor bioavailability. In one study, cell membrane-penetrating proteins TP and TP10 were coupled to NFκB decoy DNA, which blocked the effect of interleukin-1-induced NFκB activation and IL-6 gene expression. In another study, TP10-coupled Myc decoy DNA decreased proliferative capacity of N2a cells.

Individual genes can be inserted into specific sites on plasmids, and recombinant plasmids can be introduced into living cells. A method using macro-branched Tat has been proposed for plasmid DNA delivery into various cell lines and showed significant transfection capabilities. Multimers of Tat have been found to increase transfection efficiency of plasmid DNA by 6-8 times more than poly-L-arginine or mutant Tat2-M1, and by 390 times compared with the standard vectors.

The development of novel therapeutic proteins to treat diseases is limited by low efficiency of traditional delivery methods. Recently, several methods using cell membrane-penetrating proteins as vehicles to deliver biologically active, full-length proteins into living cells and animals have been reported.

Several groups have successfully delivered cell membrane-penetrating protein-fused proteins in vitro. Tat was able to deliver different proteins, such as horseradish peroxidase and RNase A across cell membrane into the cytoplasm in different cell lines in vitro. The size range of proteins with effective delivery is from 30 kDa to 120-150 kDa. In one study, Tat-fused proteins are rapidly internalized by lipid raft-dependent macropinocytosis using a transducible Tat-Cre recombinase reporter assay on live cells. In another study, a Tat-fused protein was delivered into mitochondria of breast cancer cells and decreased the survival of breast cancer cells, which showed capability of Tat-fusion proteins to modulate mitochondrial function and cell survival. However, very few in vivo studies have succeeded. In one study, in vivo delivery of Tat- or penetratin-crosslinked Fab fragments yielded varied organ distributions and an overall increase in organ retention, which showed tissue localization.

A non-covalent method that forms cell membrane-penetrating protein/protein complexes has been developed to address the limitations in covalent methods, such as chemical modification before crosslinking and denaturation of proteins before delivery. In one study, a short amphipathic peptide carrier, Pep-1, and protein complexes have proven effective for delivery. It was shown that Pep-1 could facilitate rapid cellular uptake of various peptides, proteins, and even full-length antibodies with high efficiency and less toxicity. This approach has greatly simplified the formulation of reagents.

Cell membrane-penetrating proteins have been reported for use as transporters of contrast agents across plasma membranes. These contrast agents are able to label the tumor cells, making the compounds important tools in cancer diagnosis; they are also used in in vivo and in vitro cellular experiments. Improvements for the widely-used Tat arginine-rich substrate include the usage of unnatural β or γ amino acids. This strategy offers multiple advantages, such resistance to proteolytic degradation, a natural degradation process by which peptide bonds are hydrolyzed to amino acids. Unnatural acid insertion in the peptide chain has multiple advantages. It facilitates the formation of stable foldamers with distinct secondary structure. β-Peptides are conformationally more stable in aqueous solution than naturally occurring peptides, especially for small amino acid chains. The secondary structure is reinforced by the presence of a rigid β-amino acid, which contains cyclohexane or cyclopentane fragments. These fragments generate a more rigid structure and influence the opening angle of the foldamer. These features are very important for new peptide design. Helical β-peptides mimic antimicrobial activities of host defense peptides, a mimicry which requires the orientation of cationic-hydrophilic on one side, and hydrophobic residues on the other side of the helix. The attachment of fluorescent group to one head of the molecule confers contrast properties.

A new strategy to enhance the cellular up-take capacity of cell membrane-penetrating protein is based on association of polycationic and polyanionic domains that are separated by a linker. Cellular association of polycationic residues (polyarginine) with negatively-charged membrane cells is effectively blocked by the presence of polyanionic residue (poly-glutamic acid) and the linker, which confer the proper distance between these two charged residues in order to maximize their interaction. These peptides adopt hairpin structure, confirmed by Overhauser effect correlation for proton-proton proximities of the two charged moieties. At this stage only the linker is exposed to protease hydrolysis in vivo applications. The linker hydrolysis occur and the two charged fragments experience more conformational freedom. In the absence of linker, the cationic peptide can interact more efficiently with the target cell and cellular uptake occurs before proteolysis. This strategy found applications in labeling tumor cells in vivo. Tumor cells were marked in minutes. Linker degradation can be predicted by the amount of D-aminoacids (the unnatural isomer) incorporated in the peptide chain, this restricts in vivo proteolysis to the central linker.

The presence of octamer arginine residues allows cell membrane transduction of various cargo molecules including peptides, DNA, siRNA, and contrast agents. However, the ability of cross membrane is not unidirectional; arginine-based cell membrane-penetrating proteins are able to enter and exit the cell membrane, displaying an overall decreasing concentration of contrast agent and a decrease of magnetic resonance (MR) signal in time. This limits their application in vivo. To solve this problem, contrast agents with a disulfide, reversible bond between metal chelate and transduction moiety enhance the cell-associated retention. The disulfide bond is reduced by the target cell environment and the metal chelate remains trapped in the cytoplasm, increasing the retention time of chelate in the target cell.

To be effective in membrane penetration, the peptide may contain at least five arginines. The peptide composition and potential mechanism for penetration is well described in several papers (Wender et al., Proc. Natl. Acad. Sci. U.S.A. 97:13003-13008 (2000) and Fuchs and Raines, Protein Science 14:1538-1544 (2005)) that are also described in Wikipedia (search for cell penetrating peptides).

Mitchell et al., Chemical Biology & DrugDesign 56(5:318-325 (2000) disclosed a comparison of relative cell penetrating abilities of peptides containing a stretch of 3, 5, 7, 9, or 11 Arginines. Importantly, charge alone is not sufficient for cell membrane penetration as poly-histidine, -lysine, or -ornithine did not exhibit the same membrane penetration activity as did poly-arginine. Wender et al., Proc. Natl. Acad. Sci. U.S.A. 97:13003-13008 (2000). Cell membrane penetrating peptides are reviewed in Heitz et al., Br. J. Pharm. 157:195-206 (2009).

It has been reported that when a polypeptide consisting of 250 Arginine (R) residues penetrating the cell membrane. Farber et al., BBA 390:298-311 (1975). Others have demonstrated that either a peptide having a stretch of 10 or 11 Arginines or a Tat-peptide derivative may be employed to achieve penetration through a cell membrane of a human CD34+ cell by attachment of a Tat-peptide variant to nanoparticles. Lewin et al., Nat. Biotechnology 18:410-414 (2000).

Cell membrane-penetrating molecules can be attached directly to one or more functional groups that are: (1) attached directly to the surface of a nanoparticle, (2) attached to or associated with a polymer coating that encapsulates the nanoparticle, (3) attached to or associated with a lipid bilayer that encapsulates the nanoparticle, (4) attached to one or more bioactive molecules, and/or (5) part of a fusion protein that comprises both a bioactive molecule and a cell membrane-penetrating molecule.

Cell membrane-penetrating molecules can also be attached indirectly to one or more functional groups through a crosslinking agent, such as a bifunctional crosslinking agent, that attaches to the cell membrane-penetrating molecule through one functional group and directly to one or more functional groups that are attached as described in (1)-(5) of the preceding paragraph.

Alternatively, molecules that penetrate a mammalian cell membrane can be attached to one or more crosslinking agents that include one or more functional groups that can: (1) attach to a functional group on the surface of a nanoparticle, (2) attach to a functional group on a polymer coating that encapsulates the nanoparticle, (3) attach to a functional group on a crosslinking agent that is attached to a functional group on the surface of a nanoparticle, (4) attach to a crosslinking agent that is attached to a functional group on a polymer coating that encapsulates the nanoparticle, (5) attach to one or more bioactive molecule, such as one or more bioactive molecules that can modulate one or more cellular functions, which bioactive molecule is attached to a nanoparticle via one or more crosslinking agents or functional groups.

Peptides or polypeptides that penetrate through a mammalian cell membrane may be from about five amino acids to about 100 amino acids, or from about five amino acids to about 50 amino acids, or from about five amino acids to about 25 amino acids, or from about five amino acids to about nine amino acids.

Peptides or polypeptides that penetrate through a mammalian cell membrane may include from about five basic amino acids to about 100 basic amino acids, or from about five basic amino acids to about 50 basic amino acids, or from about five basic amino acids to about 25 basic amino acids, or from about five basic amino acids to about nine basic amino acids. In some embodiments, whereas in other embodiments the peptide includes nine basic amino acids.

U.S. Patent Publication Nos. 2005/0106625, 2006/0246426, 2006/0286142 and PCT Patent Publication No. WO 2007/050130 describe methods for attaching polypeptides to a gold nanoparticle core by employing fusion proteins that include a polypeptide of interest and one to seven repeats of a high affinity gold binding peptide.

Any peptide-based molecule may be added to the solution containing a certain amount of ethylene glycol for freezing at −30° C. Per 3 micrograms of the protein in 14 μl solution, add 10 μl of a freshly-prepared DTT (dithiothreitol, Cleland's reagent) solution in PBS with vigorous vortexing.

Because the proteins usually contain more than one cysteine, there is a tendency to undesirably crosslink different molecules. Therefore, the excess DTT reduces the dithiol linkage. Reaction is allowed to proceed for two hours at 4° C. and then excess reagent is removed by an Amicon centrifugal filter unit with a 3,000 MW cutoff. The activated nanoparticles and the protein solutions are combined and allowed to react for two hours, after which the unreacted protein is removed by an Amicon centrifugal filter unit having an appropriate MW cutoff. Instead of Amicon spin filter columns, small spin columns containing solid size filtering components, such as Bio-Rad P columns can also be used.

Reaction of amino-functionalized nanoparticle cores with a targeting molecule, such as a cell membrane-penetrating molecule, or a biologically active molecule can be achieved by reacting with N-succinimidyl 3-(2-pyridyldithio)propionate (SPDP). Amino functionalized nanoparticle cores can be suspended in 0.1 M phosphate buffer, pH 7.4, and 2 mL of 25 mM SPDP in DMSO (50 umoles SPDP). The mixture was allowed to stand for 60 min at room temperature. Low molecular impurities were removed by PD-10 columns (Sigma Chemical, St. Louis, Mo.) equilibrated with 0.01M Tris and 0.02M citrate, pH 7.4 buffer. The number of amine groups can be obtained for the amount of 2PT released assayed by addition of dithiothreitol. Zhao, Bioconjug. Chem. 13:840-4 (2002).

N-hydroxysuccinimide may be reacted with the free amine groups on a nanoparticle core in order to form a maleimide end group that can react with cysteines on a targeting molecule or a biologically active molecule. Intramolecular disulfide bond formation may be controlled by first reducing a targeting molecule or a biologically active molecule with Cleland's reagent or other reducing agent. A purified targeting molecule or a biologically active molecule may be reacted with the nanoparticle cores containing the LC-maleimide group followed by spin filtration to remove reactants (e.g., an Amicon spin filter with 50K cutoff). Surface amine groups on nanoparticle cores may be converted to sufhydryl groups in a reaction with Traut's reagent or to carboxylic acids with iodoacetic acid.

During the optimization, the membrane-permeable peptide and the proteins will be mixed at different ratios to achieve the maximum number of molecules coupled to the nanoparticle. Based on previously published studies, 3-4 molecules of surface-bound cell penetrating peptide per nanoparticle are sufficient for efficient intracellular delivery of superparamagnetic nanoparticles.

The use of LC2-extender arm provides an important means for increasing the number of bound peptide-based molecules. Using varying concentrations of NHS-LC-SPDP allows increased number of anchored peptide and protein molecule to the surface of nanoparticles. This increase permits improved penetration efficiency and more robust cell reprogramming activity.

A6. Biologically Active Molecules

Functionalized nanoparticles of the present disclosure include one or more biologically active molecule(s) that introduce one or more new function(s) to a cell or regulate, modulate, and/or normalize one or more cellular function(s) such as cell maintenance/survival, cell growth/proliferation, cell differentiation, and/or cell death. Within certain aspects, biologically active molecules include, but are not limited to antibodies, full-length proteins, polypeptides, and/or peptides; nucleic acids, such as cDNAs, RNAs, oligonucleotides, primers, and probes; and/or small molecules that can regulate, modulate, normalize, provide, and/or restore one or more cellular function(s), such as cell maintenance, survival, growth/proliferation, differentiation, and/or death.

In general, a biologically active molecule is a synthetic or natural molecule that specifically binds or otherwise links to, e.g., covalently or non-covalently binds to or hybridizes with, a target molecule within a cell, or with another binding moiety- or aggregation-inducing molecule. For example, a biologically active molecule can be a synthetic oligonucleotide that hybridizes to a specific complementary nucleic acid target.

A biologically active molecule can also be a polysaccharide that binds to a corresponding target. In certain embodiments, the binding moieties can be designed or selected to serve, when bound to another binding moiety, as substrates for a target molecule such as an enzyme in solution. Binding moieties include, for example, oligonucleotide binding moieties, polypeptide binding moieties, antibody binding moieties, and polysaccharide binding moieties.

U.S. Patent Publication No. 2006/0251726 describes nanoparticle-polypeptide complexes that include a biologically active polypeptide, such as a tumor suppressor protein, in association with a nanoparticle, wherein the biologically active polypeptide is modified by the addition of a chemical moiety that facilitates cellular uptake of the protein.

Within certain embodiments, biologically active molecules include molecules that can promote the differentiation of a cell into an induced cardiomyocyte-like cell (iCM). Suitable cardiomyocyte inducing agents include Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and c-Myc, or a functional domain or structural variant thereof.

Within certain embodiments, biologically active molecules include molecules that can induce the reprogramming of a somatic cell, such as a fibroblast, into a dedifferentiated cell type, such as a pluripotent stem cell (referred to herein as nanoparticle induced pluripotent stem cells or niPSCs). Suitable biologically active molecules for reprogramming somatic cells include, for example, transcription factors such as the transcription factors Oct4, Sox2, Nanog, Lin28, cMyc, and Klf4, which provide an integral regulatory function to a cell and promote the dedifferentiation of cells, such as fibroblasts, to stem cells, in particular pluripotent stem cells (PSCs) such as induced pluripotent stem cells (iPSCs).

Within related embodiments, biologically active molecules include molecules that can promote the differentiation of cells into induced pluripotent stem cells (iPSCs). Suitable stem cell inducing agents include Lin28, Nanog, Mir-302bcad/367, Mir-302, Mir-200c, Mir-369, Oct4, Sax2, Klf4, and c-Myc, or a functional domain or structural variant thereof.

Within certain embodiments, biologically active molecules include molecules that can promote the differentiation of cells into induced neuronal cells (iNCs). Suitable neuronal cell inducing agents include Brn2, Asc11, Myt11, Zic1, Mir-9, Mir-124, NeuroD1, Lmx1a, FoxA2, Oct4, Sox2, Klf4, c-Myc, or a functional domain or structural vaiant thereof.

Within certain embodiments, biologically active molecules include molecules that can promote the differentiation of cells into other types of induced cells. Suitable inducing agents include those presented in Table 1.

Within certain embodiments, biologically active molecules include molecules that can promote the repair of a genetic mutation in a target nucleic acid. Suitable biologically active molecules include guide nucleic acids that are specific for a target nucleic acid, (ii) nucleases that cleave a target nucleic acid upon binding of a guide nucleic acid to the target nucleic acid, and (iii) nucleic acids that encodes a nuclease that cleaves a target nucleic acid upon binding of a guide nucleic acid to a target nucleic acid.

Gold nanoparticles have also shown potential as intracellular delivery vehicles for antisense oligonucleotides (ssDNA, dsDNA) by providing protection against intracellular nucleases and improving ease of functionalization for selective targeting. Recently, Conde et al developed a theranostic system capable of intersecting all RNA pathways—from gene specific downregulation to regulating the siRNA and miRNA gene-expression-silencing pathways. The authors reported the development gold nanoparticles functionalized with a fluorophore labeled hairpin-DNA, i.e. gold nanobeacons, as an innovative theranostic approach for detection and inhibition of sequence-specific DNA and RNA for in vitro and ex vivo applications. These gold nanobeacons are capable of efficiently silencing single gene expression, exogenous siRNA and endogenous miRNAs while yielding a quantifiable fluorescence signal directly proportional to the level of silencing. Under hairpin configuration, proximity to gold nanoparticles leads to fluorescence quenching; hybridization to a complementary target to conformational reorganization of the gold nanobeacons, restoring fluorescence emission duwhen the fluorophore and the gold nanoparticle part from each other. This concept can easily be extended and adapted to assist in vitro evaluation of ex vivo gene and RNAi silencing potentials of a given sequence with the ability to monitor real-time gene delivery action. An siRNA may be conjugated to a gold nanoparticle covalently by use of thiolated siRNA or ionical through the interaction of the negatively charged siRNA to the modified surface of the AuNP.

When attaching molecules, including targeting molecules and biologically active molecules, to a nanoparticle core, residual and active groups of molecules that were attached previously may interfere with the coupling chemistries. Thus, permanent or reversible capping reagents may be advantageously used to block these active moieties from interference when attaching a second or third protein to the nanoparticle core.

Numerous different capping compounds may be used to block the unreacted moiety. One skilled in the art will appreciate that capping compounds may interfere with protein activity and, therefore, may be used selectively and/or sparingly. Capping compounds are used most often when a second chemical attachment step is required and this functional group may interfere. Exemplary suitable capping and blocking reagents include Citraconic Anhydride (specific for NH), Ethyl Maleimide (specific for SH), and Mercaptoethanol (specific for maleimide).

### B. Methods for Making Functionalized Nanoparticles

The present disclosure provides methods for making functionalized nanoparticles, including functionalized superparamagnetic nanoparticles, functionalized polymeric nanoparticles, and functionalized gold nanoparticles, which are capable of penetrating through a mammalian cell membrane and delivering intracellularly one or more biologically active molecules for affecting and/or introducing one or more cellular function.

Within certain aspects, these methods include, in various combination and order: (1) providing a nanoparticle core having one or more functional groups attached directly thereto or associated therewith; (2) attaching one or more biologically active molecule(s) for effectuating one or more cellular functions via a functional group that is attached to or associated with the biologically active molecule(s) to a functional group that is attached to and/or associated with the nanoparticle core; and (3) attaching one or more cell membrane-penetrating molecule(s) via a functional group that is attached to or associated with the cell membrane-penetrating molecule(s) to a functional group that is attached to and/or associated with the nanoparticle core.

Within other aspects, these methods include, in various combination and order: (1) providing a nanoparticle core having one or more functional groups attached directly thereto or associated therewith; (2) attaching via a first functional group one or more crosslinking agent(s), each having a first functional group and a second functional group, to one or more of functional group(s) attached to and/or associated with a nanoparticle core; (3) attaching one or more biologically active molecule(s) for effectuating one or more cellular functions via a functional group that is attached to or associated with the biologically active molecule(s) to a second functional group on the crosslinking agent; and (4) attaching one or more cell membrane-penetrating molecule(s) via a functional group that is attached to or associated with the cell membrane-penetrating molecule(s) to a second functional group on the crosslinking agent.

Within other aspects, these methods include, in various combination and order: (1) providing a nanoparticle core; (2) encapsulating the nanoparticle core with a polymer coating or a lipid bilayer, wherein the polymer coating or lipid bilayer has one or more functional groups attached thereto or associated therewith; (3) attaching one or more biologically active molecule(s) for effectuating one or more cellular functions via a functional group that is attached to or associated with the biologically active molecule(s) to a functional group that is attached to and/or associated with the polymer coating or lipid bilayer; and (4) attaching one or more cell membrane-penetrating molecule(s) via a functional group that is attached to or associated with the cell membrane-penetrating molecule(s) to a functional group that is attached to and/or associated with the polymer coating or lipid bilayer.

Within other aspects, these methods include, in various combination and order: (1) providing a nanoparticle core; (2) encapsulating the nanoparticle core with a polymer coating or a lipid bilayer, wherein the polymer coating or lipid bilayer has one or more functional groups attached thereto or associated therewith; (3) attaching via a first functional group one or more crosslinking agent(s), each having a first functional group and a second functional group, to one or more of functional group(s) attached to and/or associated with the polymer coating or lipid bilayer; (4) attaching one or more biologically active molecule(s) for effectuating one or more cellular functions via a functional group that is attached to or associated with the biologically active molecule(s) to a second functional group on the crosslinking agent; and (5) attaching one or more cell membrane-penetrating molecule(s) via a functional group that is attached to or associated with the cell membrane-penetrating molecule(s) to a second functional group on the crosslinking agent.

Suitable nanoparticle cores that may be employed in each of these embodiments include metallic, ceramic, and synthetic nanoparticle cores having hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Metallic nanoparticle cores include magnetic nanoparticles, including iron-containing nanoparticle cores, such as paramagnetic nanoparticle cores and superparamagnetic nanoparticle cores; polymeric nanoparticle cores; gold nanoparticle cores; as well as nanoparticle cores made with one or more additional metals including any one of, or combination of two or more of, aluminum, barium, beryllium, chromium, cobalt, copper, iron, manganese, magnesium, strontium, zinc, rare earth metal, or trivalent metal ion. Other metal species, such as silicon oxide, silver, titanium, and ITO can also be used in the presently disclosed nanoparticle cores.

Suitable polymer coatings or lipid bilayers that may be used in the functionalized nanoparticles disclosed herein include, for example, those polymer coatings or lipid bilayers that (1) reduce nanoparticle cytotoxicity, (2) increase nanoparticle hydrophilicity or hydrophobicity, and/or (3) to provide a surface that can be modified with one or more functional groups for attachment to one or more crosslinking agents, biologically active molecules, and/or cell membrane-penetrating molecules.

Suitable functional groups that may be used in the functionalized nanoparticles disclosed herein include, for example, amino groups (—NH₂), sulfhydryl groups (—SH), carboxyl groups (—COOH), guanidyl groups (—NH₂—C(NH)—NH₂), hydroxyl groups (—OH), azido groups (—N₃), and/or carbohydrates. Such functional groups can attach directly to a biologically active molecule, a cell membrane-penetrating molecule, and/or a crosslinking agent through, for example, an amino, sulfhydryl, or phosphate group. Alternatively, a functional group can be provided as a functionalized polymer that is formed, for example, on a synthetic nanoparticle shell.

Functional groups may also include one or more stabilizing groups, such as stabilizing groups selected from the group consisting of phosphate, diphosphate, carboxylate, polyphosphate, thiophosphate, phosphonate, thiophosphonate, sulphate, sulphonate, mercapto, silanetriol, trial koxysilane-containing polyalkylene glycols, polyethylene glycols, carbohydrate or phosphate-containing nucleotides, oligomers thereof or polymers thereof.

Suitable crosslinking agents that may be used in the functionalized nanoparticles disclosed herein include long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pypridyldithio)-proprionate (SPDP); long-chain N-Succinimidyl-3-(pypridyldithio)-proprionate (LC-SPDP); sulfo-N-Succinimidyl-3-(pypridyldithio)-proprionate (sulfo-SPDP); long-chain sulfo-N-Succinimidyl-3-(pypridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pypridyldithio)-proprionate polyethylene glycolₘ (SPDP(PEG)ₘ); and sulfo-N-Succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

Suitable biologically active molecules that may be used in the functionalized nanoparticles disclosed herein include one or more biologically active molecule(s) that introduce one or more new function(s) to a cell or regulate, modulate, and/or normalize one or more cellular function(s) such as cell maintenance/survival, cell growth/proliferation, cell differentiation, and/or cell death. Within certain aspects, biologically active molecules include, but are not limited to antibodies, full-length proteins, polypeptides, and/or peptides; nucleic acids, such as cDNAs, RNAs, oligonucleotides, primers, and probes; and/or small molecules that can regulate, modulate, normalize, provide, and/or restore one or more cellular function(s), such as cell maintenance, survival, growth/proliferation, differentiation, and/or death.

Suitable targeting molecules that may be used in the functionalized nanoparticles disclosed herein include, for example, full-length proteins, polypeptides, and/or peptides; nucleic acids, such as cDNAs, RNAs, oligonucleotides, primers, and/or probes; and/or small molecules to facilitate the specific delivery of a functionalized nanoparticle to a target cell. Targeting molecules include cell membrane-penetrating molecules, which facilitate the (i) the cellular uptake of a functionalized nanoparticle through a mammalian cell plasma membrane and, optionally, (ii) the subcellular localization of a functionalized nanoparticle into, for example, a mammalian cell nucleus, mitochondria, endosome, lysosome, or other organelle via a mammalian cell nuclear membrane, mitochondrial membrane, lysosomal membrane, endosomal membrane, and/or other organelle membrane.

Suitable cell membrane-penetrating molecules that may be used in the functionalized nanoparticles disclosed herein include full-length proteins, polypeptides, peptides, nucleic acids, and small molecules. Exemplary peptides include those deriving from HIV Tat as well as peptides having from five to nine or more basic amino acids, such as lysine and arginine, and include peptides having from five to nine or more contiguous basic amino acids, such as lysine and arginine.

The present disclosure further provides methods for manufacturing a functionalized nanoparticle for promoting the differentiation of a cell into an induced cardiomyocyte-like cell (iCM), which methods include attaching a cardiomyocyte inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and c-Myc, or a functional domain or structural variant thereof.

The present disclosure further provides methods for manufacturing functionalized nanoparticles for promoting the differentiation of cells into induced pluripotent stem cells (iPSCs), which methods include attaching a stem cell inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable stem cell inducing agents include Lin28, Nanog, Mir-302bcad/367, Mir-302, Mir-200c, Mir-369, Oct4, Sox2, Klf4, and o-Myc, or a functional domain or structural variant thereof.

The present disclosure further provides methods for manufacturing functionalized nanoparticles for promoting the differentiation of cells into induced neuronal cells (iNCs), which methods include attaching a neuronal cell inducing agent and a cell targeting molecule to a nanoparticle core, including a metal nanoparticle core, such as an iron or gold containing nanoparticle core, a synthetic nanoparticle core, or a ceramic nanoparticle core. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable neuronal cell inducing agents include Brn2, Asc11, Myt11, Zic1, Mir-9, Mir-124, NeuroD1, Lmx1a, FoxA2, Oct4, Sox2, Klf4, c-Myc, or a functional domain or structural variant thereof.

The present disclosure further provides methods for manufacturing functionalized nanoparticles for promoting the repair of a genetic mutation in a target nucleic acid, which methods include attaching to a nanoparticle core, including a metal nanoparticle core, such as an iron or gold containing nanoparticle core, a synthetic nanoparticle core, or a ceramic nanoparticle core: (a) a biologically active molecule selected from (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including a cell membrane-penetrating molecule, such as an HIV Tat derived peptide or other peptide having, for example, from five to nine basic amino acids, including arginine and/or lysine. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

An exemplary scheme for making functionalized superparamagnetic iron oxide nanoparticles (SPIONs) that include one or more protein and one or more peptide separated from the core SPION by one or more bi-functional crosslinking agent is presented in FIGS. 1A and 1B. In this example, an amino-SPION is reacted with a long-chain N-hydroxysuccinimide (NHS)N-succinimidyl-3-(pypridyldithio)-proprionate (NHS-LC-SPDP) linker (step I) followed by a reduction step (step II) to generate a SPION having a long-chain Crosslinking agent with a reactive sulfhydryl (—SH) group (SH-LC-amino-SPION; FIG. 1A).

In separate reactions, a protein is reacted with a long-chain (LC1) N-hydroxysuccinimide (NHS) succinimidyl 4-(N-maleimidomethyl) cyclohexane-1-carboxylate (SMCC; NHS-LC1-SMCC; step III) and a peptide is reacted with another long-chain (LC2) N-hydroxysuccinimide (NHS) succinimidyl 4-(N-maleimidomethyl) cyclohexane-1-carboxylate (SMCC; NHS-LC2-SMCC; step IV) to yield a protein and peptide having a reactive N-maleimidomethyl group that, which reacted with the sulfhydryl group on the HS-LC-HN-SPION (step V), yields a peptide and protein conjugated, functionalized nanoparticle (in this case a bioactive SPION) according to the present disclosure (FIG. 1B).

Different proteins may contain the same functional groups, making it difficult to label the nanoparticle with a variety of proteins or peptides. Certain reagents exist, however, to permit the desired change in functional groups and provide desired selectivity in a stepwise fashion without interference from the other proteins. Such reagents include, for example, SPDP, which can be used to convert and amine to a sulfhydryl, biasing receptivity towards reaction with a maleimide moiety.

Superparamagnetic nanoparticles, including superparamagnetic iron oxide nanoparticles (SPION), can be functionalized with amino-groups on the exterior (amino-SPION) as described in Ma et al., J. Nanopart. Res. 13:3249-3257 (2011) and can be obtained commercially from various sources (e.g., Nano Diagnostics, Fayetteville, Ark.; Skyspring Nanomaterials, Houston, Tex.; Sigma-Aldrich).

The methods disclosed herein may utilize biocompatible nanoparticle cores, including for example, superparamagnetic iron oxide, gold nanoparticle cores, or polymeric nanoparticle cores similar to those previously described in scientific literature. See, Lewin et al., Nat. Biotech. 18:410-414, (2000); Shen et al., Magn. Reson. Med. 29:599-604 (1993); and Weissleder et al., Am. J. Roentgeneol. 152:167-173 (1989). Such nanoparticles can be used, for example, in clinical settings for magnetic resonance imaging of bone marrow cells, lymph nodes, spleen and liver. See, e.g., Shen et al., Magn. Reson. Med. 29:599 (1993) and Harisinghani et al., Am. J. Roentgenol. 172:1347 (1999).

Magnetic iron oxide nanoparticles sized less than 50 nm and containing cross-linked cell membrane-permeable TAT-derived peptide efficiently internalize into hematopoietic and neural progenitor cells in quantities of up to 30 pg of superparamagnetic iron nanoparticles per cell. Lewin et al., Nat. Biotech. 18:410-414, (2000). Furthermore, nanoparticle incorporation does not affect proliferative and differentiation characteristics of bone marrow-derived CD34+ primitive progenitor cells or the cell viability. Lewin et al., Nat. Biotech. 18:410-414, (2000). Accordingly, the disclosed nanoparticles can be used for in vivo tracking of the labeled cells. The labeled cells retain their differentiation capabilities and can also be detected in tissue samples using magnetic resonance imaging.

Disclosed herein are nanoparticle-based compositions, which are functionalized to carry various sets of RNAs (including mRNAs, microRNAs, and siRNAs), proteins, peptides and other small molecules that can serve as excellent vehicles for intracellular delivery of biologically active molecules to target intracellular events and modulate cellular function and properties for direct reprogramming of human somatic cells into various cell types of interest.

### C. Functionalized Nanoparticles for Cellular Reprogramming

Within certain embodiments, the present disclosure provides functionalized nanoparticles that may be used to reprogram a cell, such as a somatic cell or a stem cell, to a cell having desired phenotypic characteristics. Such functionalized nanoparticles include a nanoparticle core, one or more cell targeting molecule(s), and one or more biologically active molecules to affecting and/or introducing one or more cellular functionalities.

Functionalized nanoparticles according to these embodiments may employ metallic nanoparticle cores, ceramic nanoparticle cores, or synthetic nanoparticle cores. Exemplary suitable metallic nanoparticle cores include (1) iron containing nanoparticle cores, such as paramagnetic nanoparticle cores and superparamagnetic nanoparticle cores, (2) gold nanoparticle cores, and (3) polymeric nanoparticle cores. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

Functionalized nanoparticles for cellular reprogramming, including direct cellular reprogramming, may include a polymer coating or lipid bilayer that (1) reduces nanoparticle cytotoxicity, (2) increases nanoparticle hydrophilicity or hydrophobicity, and/or (3) provides a surface that can be modified with one or more functional groups for attachment to one or more crosslinking agents, biologically active molecules, and/or cell targeting molecules.

Nanoparticle cores, polymer coatings, and/or lipid bilayers may include one or more functional groups including, for example, one or more amino groups (—NH₂), sulfhydryl groups (—SH), carboxyl groups (—COOH), guanidyl groups (—NH₂—C(NH)—NH₂), hydroxyl groups (—OH), azido groups (—N₃), and/or carbohydrates.

Nanoparticle cores, polymer coatings, and/or lipid bilayers may include one or more stabilizing groups including, for example, one or more phosphate, diphosphate, carboxylate, polyphosphate, thiophosphate, phosphonate, thiophosphonate, sulphate, sulphonate, mercapto, silanetriol, trialkoxysilane-containing polyalkylene glycol, polyethylene glycol, a carbohydrate, and a phosphate-containing nucleotide.

Functionalized nanoparticles for cellular reprogramming may include one or more cross-linking agents including, for example, one or more long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pypridyldithio)-proprionate (SPDP); long-chain N-Succinimidyl-3-(pypridyldithio)-proprionate (LC-SPDP); sulfo-N-Succinimidyl-3-(pypridyldithio)-proprionate (sulfo-SPDP); long-chain sulfo-N-Succinimidyl-3-(pypridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pypridyldithio)-proprionate polyethylene glycolₘ (SPDP(PEG)ₘ); and sulfo-N-Succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

Cell targeting molecules employed in certain of the functionalized nanoparticles for cellular reprogramming disclosed herein are cell membrane-penetrating molecules that facilitate the cellular uptake of the functionalized nanoparticle via a mammalian cell plasma membrane and, in certain aspects, may also facilitate the subcellular localization of the functionalized nanoparticle into a mammalian cell nucleus, a mitochondrion, a lysosome, an endosome, or another organelle. As disclosed herein, cell membrane-penetrating molecules may be full-length proteins, polypeptides, peptides, cDNAs, mRNAs, siRNAs, shRNAs, microRNAs, oligonucleotides, and/or small molecules. For example, a suitable cell membrane-penetrating molecule may comprise five to nine basic amino acids, in particular five to nine contiguous basic amino acids selected from lysine and arginine.

Biologically active molecules employed in certain of the functionalized nanoparticles for cellular reprogramming disclosed herein can regulate, modulate, normalize, provide, and/or restore one or more cellular function(s), such as cell maintenance, survival, growth/proliferation, differentiation, and/or death. As disclosed herein, biologically active molecules may be full-length proteins, polypeptides, peptides, cDNAs, mRNAs, siRNAs, shRNAs, microRNAs, oligonucleotides, and/or small molecules.

Biologically active molecules that may be advantageously employed in functionalized nanoparticles for cellular reprogramming include inducing agents, such as transcription factors, as exemplified by the biologically active molecules for use in direct reprogramming that are presented in Table 1.

TABLE 1
Illustrative Reprogramming Factors and Combinations

| INDUCED PLURIPOTENT STEM CELLS | | | |
|---|---|---|---|
| (iPSC) | | CARDIOMYOCYTES | |
| TD | | TD | |
| FACTOR | REFERENCE | FACTOR | REFERENCE |
| Oct4 | Takahashi et al., “Induction of | Tbx5 | Ieda et al., “Direct |
| Sox2 | pluripotent stem cells from | Mef2c | reprogramming of fibroblasts |
| c-Myc | adult human fibroblasts by | Gata-4 | into functional cardiomyocytes |
| Klf4 | defined factors” Cell 131: 861- | Mesp1 | by defined factors” Cell |
| | 872 (2007) | | 142: 375-386 (2010) |
| Lin28 | Yu et al., “Induced pluripotent | Mir-1-1 | Ivey et al., “MicroRNA |
| Nanog | stem cell lines derived from | | regulation of cell lineages in |
| | human somatic cell” Science | | mouse and human embryonic |
| | 318: 1917-2920 (2007) | | stem cells” Cell Stem Cell |
| | | | 2: 219-229 (2008) |
| Mir- | Anokye-Danso et al., “Highly | Oct4 | Efe et al., “Conversion of |
| 302bcad/367 | efficient miRNA-mediated | Sox2 | mouse fibroblasts into |
| | reprogramming of mouse and | Klf4 | cardiomyocytes using a direct |
| | human somatic cells to | c-Myc | reprogramming strategy” Nat. |
| | pluripotency” Cell Stem Cell | | Cell Biol. 13: 215-222 (2011) |
| | 8: 376-388 (2011) | | |
| Mir-302 | Miyoshi et al., | CHIR99021 | Cao et al., “Conversion of |
| Mir-200c | “Reprogramming of mouse and | A83-01 | human fibroblasts into |
| Mir-369 | human cells to pluripotency | BIX01294 | functional cardiomyocytes by |
| | using mature microRNAs” Cell | AS8351 | small molecules” Science |
| | Stem Cell 8: 633-638 (2011) | SC1 | 352: 1216-1220 (2016) |
| | | Y27632 | |
| | | OAC2 | |
| | | SU16F | |
| | | JNJ10198409 | |

NEURONS

| TD | | TD | |
|---|---|---|---|
| FACTOR | REFERENCE | FACTOR | REFERENCE |
| Brn2 | Vierbuchen et al., “Direct | Brn2 | Pang et al., “Induction of |
| Ascl1 | conversion of fibroblasts to | Ascl1 | human neuronal cells by |
| Mytl1 | functional neurons by defined | Mytl1 | defined transcription factors” |
| Zic1 | factors” Nature 463: 1035-1041 | NeuroD1 | Nature 476: 220-223 (2011) |
| | (2010) | | |
| Mir-9 | Yoo et al., “MicroRNA- | Ascl1 | Caiazzo et al., “Direct |
| Mir-124 | mediated conversion of human | Brn2 | generation of functional |
| Ascl1 | fibroblasts to neurons” Nature | Mytl1 | dopaminergic neurons from |
| Mytl1 | 476: 228-231 (2011) | Lmx1a | mouse and human fibroblasts” |
| | | FoxA2 | Nature 476: 224-227 (2011) |
| Mytl1 | Ambasudhan et al., “Direct | Oct4 | Kim et al., “Direct |
| Brn2 | Reprogramming of Adult | Sox2 | reprogramming of mouse |
| Mir-124 | Human Fibroblasts to | Klf4 | fibroblasts to neural |
| | Functional Neurons under | c-Myc | progenitors” Proc. Natl. Acad. |
| | Defined Conditions” Cell Stem | | Sci. USA 108: 7838-7843 |
| | Cell. 9: 113-118 (2011) | | (2011) |
| DOPAMINERGIC NEURONS | | MOTOR NEURONS | |
| TD | | TD | |
| FACTOR | REFERENCE | FACTOR | REFERENCE |
| Ascl1 | Pfisterer et al., “Direct | Lhx3 | Son et al., “Conversion of |
| Brn2 | conversion of human | Ascl1 | Mouse and Human Fibroblasts |
| Mytl1 | fibroblasts to dopaminergic | Brn2 | into Functional Spinal Motor |
| Foxa2 | neurons” Proc. Natl. Acad. Sci. | Mytl1 | Neurons” Cell Stem Cell 9: 205- |
| Lmx1a | USA 108: 10343-10348 (2011) | Ngn2 | 218 (2011) |
| | | Hb9 | |
| | | Isl1 | |
| | | NeuroD1 | |

| | |
|---|---|
| HEPATOCYTES | MYOCYTES |

| TD | | TD | |
|---|---|---|---|
| FACTOR | REFERENCE | FACTOR | REFERENCE |
| Gata-4 | Huang et al., “Induction of | MyoD | Davis et al., “Expression of a single |
| HNF1-alpha | functional hepatocyte-like cells | | transfected cDNA converts fibroblasts to |
| Foxa3 | from mouse fibroblasts by | | myoblasts” Cell 51: 987-1000 (1987) |
| | defined factors” Nature | | |
| | 475: 386-389 (2011) | | |
| HNF4-alpha | Sekiya and Suzuki, “Direct | Mir-1-1 | Cordes et al., “miR-145 and miR-143 |
| Foxa1 | conversion of mouse | Mir-133 | regulate smooth muscle cell fate and |
| Foxa2 | fibroblasts to hepatocyte-like | Mir-143 | plasticity” Nature 460: 705-710 (2009) |
| Foxa3 | cells by defined factors” Nature | Mir-145 | |
| | 475: 390-393 (2011) | | |

| BLOOD PROGENITORS | | BETA CELLS | |
|---|---|---|---|
| TD | | TD | |
| FACTOR | REFERENCE | FACTOR | REFERENCE |
| Oct4 | Szabo et al., “Direct | Ngn3 | Zhou et al., “In vivo reprogramming of |
| Gata1 | conversion of human | Pdx1 | adult pancreatic exocrine cells to beta- |
| Gata2 | fibroblasts to multilineage | MafA | cells” Nature 455: 627-632 (2008) |
| Gata3 | blood progenitors” Nature | VP16 | |
| Gata-4 | 468: 523-526 (2010) | | |

OSTEOBLASTS

| TD | |
|---|---|
| FACTOR | REFERENCE |
| Mir-2861 | Ivey and Srivastava, |
| | “microRNAs as regulators of |
| | differentiation and cell fate |
| | decisions” Cell Stem Cell 7: 36- |
| | 41 (2011) |

Exemplary functionalized nanoparticles for cellular reprogramming are presented in the sections that follow.

C1. Functionalized Nanoparticles for Producing Cardiomyocyte-Like Cells (iCMs)

Some embodiments of the present disclosure provide functionalized nanoparticles for promoting the differentiation of cells into induced cardiomyocyte-like cells (iCMs).

Within certain aspects of these embodiments, functionalized nanoparticles include (a) a nanoparticle core having first and second functional groups that are associated with and/or attached directly to the nanoparticle core; (b) one or more cell targeting molecule(s), including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a cardiomyocyte inducing agent or a nucleic acid encoding a cardiomyocyte inducing agent and wherein one or more of the cell targeting molecule(s) is attached directly to the nanoparticle core via a first functional group on the nanoparticle core and one or more of the biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a cardiomyocyte inducing agent or a nucleic acid encoding a cardiomyocyte inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a cardiomyocyte inducing agent or a nucleic acid encoding a cardiomyocyte inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a cardiomyocyte inducing agent or a nucleic acid encoding a cardiomyocyte inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable cardiomyocyte inducing agents that may be employed in functionalized nanoparticles according to these embodiments include, for example, Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and c-Myc, or a functional domain or structural variant thereof. More specifically, suitable cardiomyocyte inducing agents include: (1) one or more of Gata4, MEF2C, TBX5, MESP1, Hand2, MYOCD, miR-1, and miR-133, or a functional domain or structural variant thereof, (2) one or more of Gata4, MEF2C, TBX5, MESP1, Hand2, MYOCD, miR-1, and miR-133, or a functional domain or structural variant thereof; (3) one or more of Gata4, MEF2C, TBX5, MESP1, and MYOCD, or a functional domain or structural variant thereof, or (4) one or more of Gata4, Hand2, TBX5, MYOCD, miR-1, and miR-133, or a functional domain or structural variant thereof.

C2. Functionalized Nanoparticles for Producing Pluripotent Stem Cells

Some embodiments of the present disclosure provide functionalized nanoparticles for promoting the differentiation of cells into an induced pluripotent stem cells (iPSCs).

Within certain aspects of these embodiments, functionalized nanoparticles include (a) a nanoparticle core having first and second functional groups that are associated with and/or attached directly thereto; (b) one or more cell targeting molecule(s), including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a stem cell inducing agent or a nucleic acid encoding a stem cell inducing agent and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a stem cell inducing agent or a nucleic acid encoding a stem cell inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a stem cell inducing agent or a nucleic acid encoding a stem cell inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a stem cell inducing agent or a nucleic acid encoding a stem cell inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable stem cell inducing agents that may be employed in functionalized nanoparticles according to these embodiments include, for example, Lin28, Nanog, Mir-302bcad/367, Mir-302, Mir-200c, Mir-369, Oct4, Sox2, Klf4, and o-Myc, or a functional domain or structural variant thereof. In some applications, functionalized nanoparticles include two, three, four, five, or more stem cell inducing factors each of which is independently selected from the group consisting of Lin28, Nanog, Mir-302bcad/367, Mir-302, Mir-200c, Mir-369, Oct4, Sox2, Klf4, and o-Myc, or a functional domain or structural variant thereof.

Within some embodiments, the present disclosure addresses unmet needs in the art by providing functionalized nanoparticles that may be used in methods for the treatment of inherited or acquired disorders, including methods for the treatment of neurodegenerative and hematopoietic diseases, which functionalized nanoparticles may be used to enhance pluripotent stem cells or other cell types from the subject suffering from or diagnosed with the neurodegenerative, hematopoietic, or other inherited or acquired disease.

The stem cells, their progeny, or other more specialized cells can have any genetic aberrations that underlie the neurodegenerative disease corrected to provide for ameliorative and therapeutic function in the subject. In some aspects, the present disclosure is based on the design of functionalized nanoparticles that are effective in inducing the production of stem cells (niPSCs) (see, e.g., U.S. Publication No. 2014/0342004). In some embodiments, the disclosure also incorporates a non-integrative, nanoparticle-based delivery of gene editing materials to achieve optimized corrective editing while avoiding pitfalls of genetic integration imposed by current gene editing technologies that impose a greater impact on the target genome (e.g., CRISPR/Cas9-functionalized nanoparticles as described in U.S. Patent Application No. 62/406,542, incorporated herein by reference in its entirety). The resulting pluripotent stem or other produced cell-types contain a corrected genome, retain more native intact genome characteristics, and are safer for therapeutic uses.

In one aspect, provided herein are functionalized nanoparticles that may be used in methods for the treatment of a neurodegenerative or hematopoietic or other inherited or acquired disease or condition in a subject, e.g., a human subject, comprising administering niPSC products. For example, one skilled in the art can generate numerous human pluripotent niPSC colonies from about 20,000 primary human skin fibroblasts or monocytes. Therefore, only a small sample of human skin tissue or blood cells is necessary to generate self-perpetuating colonies of stem cells that retain their pluripotent properties. Such pluripotent niPSC colonies can be further expanded to quantities of cells large enough to provide therapeutic efficacy when administered to a patient suffering from a neurodegenerative disease or other pathological condition. In one embodiment, the number of cells needed to establish therapeutic efficacy would be about 40 million cells administered either intravenously, intrathecally or intranasally. In yet another embodiment, the number of cells could be about 20, 50, 60 or 100 million cells or more infused intravenously, intrathecally or intranasally.

In one embodiment, the cells administered would be stem cells or a product thereof. In yet another embodiment, the cells would be microglia-like cells, or a product thereof. In a further embodiment, the cells administered would be macrophage-like cells, or a product thereof.

In some embodiments, an effective amount of niPSC-derived products or other cell types administered to an individual (e.g., neural or hematopoietic progenitor cell, macrophage, microglia) is an amount that, when administered in monotherapy or in combination therapy, in one or more doses, is effective to treat a disorder (e.g., neuraldegenerative or hematopoietic disorder) in an individual in need thereof. In some embodiments, an effective amount of niPSC-derived products or other cell types administered to an individual (e.g., neural or hematopoietic progenitor cell, macrophage, microglia) is an amount that, when administered to an individual in monotherapy or in combination therapy, in one or more doses, is effective to reduce an adverse symptom of a disorder in the individual. In some embodiments, an effective amount of niPSC-derived products or other cell-types administered to an individual (e.g., neural or hematopoietic progenitor cell, macrophage, microglia) is an amount that, when administered to an individual in monotherapy or in combination therapy, in one or more doses, is effective to result in an improvement in at least one neurological function in the individual.

One skilled in the art can digest the skin or other somatic tissue with collagenase and place the cells in culture for outgrowth of fibroblast cells. The fibroblast cells so generated can be treated with Nano-OSNL and further cultured and/or expanded in the presence or absence irradiated mouse embryonic fibroblasts (iMEFs) and in the presence or absence of GSK3 and MEK inhibitors and human Leukemia Inhibitory Factor LIF (2iL) or basic FGF. In the absence of anti-differentiation factors such as 2iL, the niPSC colonies spontaneously form embryoid bodies that in non-adherent plates quickly form a monolayer of embryoid bodies. In another embodiment, it would be possible to trypsinize the niPSC colonies/embryoid bodies in the presence of ROCK inhibitor to generate a monolayer of single cells on tissue culture plates.

In one aspect, nucleated somatic cells, such as skin fibroblasts or blood monocytes (e.g., about 20,000 cells), can be obtained from patients and treated with functionalized nanoparticles as described herein and elsewhere (U.S. Patent Publication No. 2014/0342004, incorporated herein by reference in its entirety) to generate pluripotent stem cells with an intact genome. The cells can be further gene corrected using editing technology such as CRISPR/Cas9-like or other gene editing approach or related gene-editing technologies. The gene editing technology can be implemented on the cells using, e.g., functionalized nanoparticles to deliver the required editing machinery to drive differentiation into microglia based on recently established and published protocols suitable for a monolayer of single cells as well as for embryoid bodies. In other aspect, the gene corrected may be performed using patients' fibroblasts or other cell types, which subsequently may be used to generate niPSC or other cell types of interest.

Oct4, Sox2, Nano, and Lin28 (OSNL) may be produced in bacteria as his-tag proteins. For those without a free sulfhydryl, one was added. The proteins were purified by affinity chromatography and linked to paramagnetic nanoparticles using sulfhydryl linking chemistry. To facilitate entry into cells, a poly-arginine peptide was added to the nanoparticles using the same chemistry. Using fluorescent-labeled nanoparticles functionalized with polyarginine, we found that 5-30 minute exposure of human fibroblasts to functionalized nanoparticles heavily labeled the cytoplasm of these cells. In similar experiments with FITC-labeled nanoparticles functionalized with Oct4 and Sox2, a 42-hour incubation fully labeled the nuclei of human fibroblasts as evidenced by co-localization of FITC-nanoparticles with blue DAPI stained nuclei.

C3. Functionalized Nanoparticles for Producing Neuronal Cells

Some embodiments of the present disclosure provide functionalized nanoparticles for promoting the differentiation of cells into induced neuronal cells (iNCs).

Within certain aspects of these embodiments, functionalized nanoparticles include (a) a nanoparticle core having first and second functional groups that are associated with and/or attached directly to the nanoparticle core; (b) one or more cell targeting molecules, including one or more a cell membrane-penetrating molecules, such as an HIV Tat derived peptide or other peptide having, for example, from five to nine basic amino acids, including arginine and/or lysine; and (c) one or more biologically active molecule(s) wherein one or more of said biologically active molecule(s) is a neuronal cell inducing agent or a nucleic acid encoding a neuronal cell inducing agent and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other aspects of these embodiments, functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a neuronal cell inducing agent or a nucleic acid encoding a neuronal cell inducing agent and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further aspects of these embodiments, functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is a neuronal cell inducing agent or a nucleic acid encoding a neuronal cell inducing agent and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further aspects of these embodiments, functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is a neuronal cell inducing agent or a nucleic acid encoding a neuronal cell inducing agent; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable neuronal cell inducing agents that may be employed in functionalized nanoparticles according to these embodiments include, for example, Brn2, Asc11, Myt11, Zic1, Mir-9, Mir-124, NeuroD1, Lmx1a, FoxA2, Oct4, Sox2, Klf4, c-Myc, or a functional domain or structural variant thereof. In some applications, functionalized nanoparticles include two, three, four, five, or more stem cell inducing factors each of which is independently selected from the group consisting of Brn2, Asc11, Myt11, Zic1, Mir-9, Mir-124, NeuroD1, Lmx1a, FoxA2, Oct4, Sox2, Klf4, c-Myc, or a functional domain or structural variant thereof, or a functional domain or structural variant thereof.

Within some embodiments, the present disclosure addresses unmet needs in the art by providing functionalized nanoparticles that may be used in methods for the treatment of inherited or acquired disorders, including methods for the treatment of neurodegenerative and hematopoietic diseases, which functionalized nanoparticles may be used to enhance pluripotent stem cells or other cell types from the subject suffering from or diagnosed with the neurodegenerative, hematopoietic, or other inherited or acquired disease.

The stem cells, their progeny, or other more specialized cells can have any genetic aberrations that underlie the neurodegenerative disease corrected to provide for ameliorative and therapeutic function in the subject. In some aspects, the present disclosure is based on the design of functionalized nanoparticles that are effective in inducing the production of stem cells (niPSCs) (see, e.g., U.S. Patent Publication No. 2014/0342004). In some embodiments, the disclosure also incorporates a non-integrative, nanoparticle-based delivery of gene editing materials to achieve optimized corrective editing while avoiding pitfalls of genetic integration imposed by current gene editing technologies that impose a greater impact on the target genome (e.g., CRISPR/Cas9-functionalized nanoparticles as described in U.S. Patent Application No. 62/406,542, incorporated herein by reference in its entirety). The resulting pluripotent stem or other produced cell-types contain a corrected genome, retain more native intact genome characteristics, and are safer for therapeutic uses.

In one aspect, provided herein are functionalized nanoparticles that may be used in methods for the treatment of a neurodegenerative or hematopoietic or other inherited or acquired disease or condition in a subject, e.g., a human subject, comprising administering niPSC products. For example, one skilled in the art can generate numerous human pluripotent niPSC colonies from about 20,000 primary human skin fibroblasts or monocytes. Therefore, only a small sample of human skin tissue or blood cells is necessary to generate self-perpetuating colonies of stem cells that retain their pluripotent properties. Such pluripotent niPSC colonies can be further expanded to quantities of cells large enough to provide therapeutic efficacy when administered to a patient suffering from a neurodegenerative disease or other pathological condition. In one embodiment, the number of cells needed to establish therapeutic efficacy would be about 40 million cells administered either intravenously, intrathecally or intranasally. In yet another embodiment, the number of cells could be about 20, 50, 60 or 100 million cells or more infused intravenously, intrathecally or intranasally.

In one embodiment, the cells administered would be stem cells or a product thereof. In yet another embodiment, the cells would be microglia-like cells, or a product thereof. In a further embodiment, the cells administered would be macrophage-like cells, or a product thereof.

In some embodiments, an effective amount of niPSC-derived products or other cell types administered to an individual (e.g., neural or hematopoietic progenitor cell, macrophage, microglia) is an amount that, when administered in monotherapy or in combination therapy, in one or more doses, is effective to treat a disorder (e.g., neuraldegenerative or hematopoietic disorder) in an individual in need thereof. In some embodiments, an effective amount of niPSC-derived products or other cell types administered to an individual (e.g., neural or hematopoietic progenitor cell, macrophage, microglia) is an amount that, when administered to an individual in monotherapy or in combination therapy, in one or more doses, is effective to reduce an adverse symptom of a disorder in the individual. In some embodiments, an effective amount of niPSC-derived products or other cell-types administered to an individual (e.g., neural or hematopoietic progenitor cell, macrophage, microglia) is an amount that, when administered to an individual in monotherapy or in combination therapy, in one or more doses, is effective to result in an improvement in at least one neurological function in the individual.

One skilled in the art can digest the skin or other somatic tissue with collagenase and place the cells in culture for outgrowth of fibroblast cells. The fibroblast cells so generated can be treated with Nano-OSNL and further cultured and/or expanded in the presence or absence irradiated mouse embryonic fibroblasts (iMEFs) and in the presence or absence of GSK3 and MEK inhibitors and human Leukemia Inhibitory Factor LIF (2iL) or basic FGF. In the absence of anti-differentiation factors such as 2iL, the niPSC colonies spontaneously form embryoid bodies that in non-adherent plates quickly form a monolayer of embryoid bodies. In another embodiment, it would be possible to trypsinize the niPSC colonies/embryoid bodies in the presence of ROCK inhibitor to generate a monolayer of single cells on tissue culture plates.

In one aspect, nucleated somatic cells, such as skin fibroblasts or blood monocytes (e.g., about 20,000 cells), can be obtained from patients and treated with functionalized nanoparticles as described herein and elsewhere (U.S. Patent Publication No. 2014/0342004) to generate pluripotent stem cells with an intact genome. The cells can be further gene corrected using editing technology such as CRISPR/Cas9-like or other gene editing approach or related gene-editing technologies. The gene editing technology can be implemented on the cells using, e.g., functionalized nanoparticles to deliver the required editing machinery to drive differentiation into microglia based on recently established and published protocols suitable for a monolayer of single cells as well as for embryoid bodies. In other aspect, the gene corrected may be performed using patients' fibroblasts or other cell types, which subsequently may be used to generate niPSC or other cell types of interest.

### D. Functionalized Nanoparticles for Repairing Genetic Mutations and Gene Editing

Some embodiments of the present disclosure provide functionalized nanoparticles for gene editing and for repairing genetic mutations in target nucleic acids. Recent technological advances with the development of TALENs or CRISPR/Cas9 systems have made gene editing and mutational corrections possible not only in cell lines, but also in primary cells and in pluripotent stem cells.

Thus, in certain aspects of these embodiments, the present disclosure provides functionalized nanoparticles that employ a CRISPR/Cas9 system for gene editing and repair of genetic mutations. See, Ohgidani, Sci. Rep. 4:4957 (2014); Chambers, Nat. Biotech. 27:275-280 (2009); Choi, Cell Rep. 2:553-567 (2012); and van Wilgenburg, PLoS One 8:e71098 (2013). It will be understood by those of skill in the art that various aspects of this approach to gene correction may be modified to generate functionalized nanoparticles that may be used advantageously in methods for eliminating mutations in target nucleic acids and, thereby, restoring a normal or wild type gene sequence.

The CRISPR/Cas9 system that is employed herein includes (1) a guide nucleic acid, such as RNA (gRNA), having (a) a nucleotide sequence that is homologous to a region of interest within a target nucleic acid and (b) a nuclease recognition sequence, such as a Cas9 nuclease recognition sequence and (2) a nuclease, such as a Cas9 nuclease. The guide nucleic acid (gRNA) binds to the target nucleic acid near the region of interest and the nuclease binds to the gRNA and creates a double-strand DNA break (DSB) near the mutation site as specified by the gRNA. Double-strand DNA breaks, in turn, activate DSB repair machinery which repair the DSB via an intracellular Non-Homologous End Joining (NHEJ) pathway thereby producing insertions and/or deletions (indels) that disrupt the targeted locus. If a donor template having homology to the targeted locus is present, the DSB can also be repaired by the homology-directed repair (HDR) pathway, which facilitates precise correction of a gene mutation of interest. See, Overballe-Petersen, Proc. Natl. Acad. Sci. USA 110:19860-19865 (2013) and Gong, Nat. Struct. Mol. Biol. 12:304-312 (2005).

High fidelity CRISPR-Cas9 variants having reduced genome-wide off-target effects that may be suitably employed in the functionalized nanoparticles disclosed herein are described in Kleinstiver et al., Nature 529(7587):490-495 (2016). Cas9 nucleases having improved specificity, and methodology for the rational design of such improved Cas9 nucleases, may be suitably employed in the functionalized nanoparticles disclosed herein and are described in Slaymaker et al., Science 351(6268):84-88 (2016).

Functionalized nanoparticles according to these embodiments include (1) a guide nucleic acid having (a) a nucleotide sequence that binds to a target nucleic acid and (b) a nuclease binding domain; (2) a nuclease (or nucleic acid encoding a nuclease) that binds to the nuclease binding domain of the guide nucleic acid and cleaves the target sequence; and, optionally, (3) a donor nucleic acid molecule at least a portion of which can be inserted into the cleavage site within the cleavage site of the target sequence.

Within some embodiments, the present disclosure provides functionalized nanoparticles for use in gene editing and/or repair of genetic mutations in target nucleic acids, which functionalized nanoparticles include (a) a nanoparticle core; (b) one or more functional group(s) that are associated with and/or attached directly to the nanoparticle; (c) one or more cell targeting molecules, including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said one or more cell targeting molecule(s) is attached directly to the nanoparticle core via a first functional group on the nanoparticle core and one or more of said biologically active molecule(s) is attached directly to the nanoparticle core via a second functional group on the nanoparticle core.

Within other embodiments, the present disclosure provides functionalized nanoparticles for promoting the repair of genetic mutations in target nucleic acids, which functionalized nanoparticles include (a) a nanoparticle core having first and second functional groups that are associated with and/or attached directly to the nanoparticle core; (b) first and second crosslinking agents, said first crosslinking agent having a first length and said second crosslinking agent having a second length, each having first and second functional groups wherein said first crosslinking agent is attached directly to the nanoparticle core via a first functional group on said nanoparticle core and a first functional group on said first crosslinking agent and wherein said second crosslinking agent is attached directly to the nanoparticle core via a first functional group on said nanoparticle core and a first functional group on said second crosslinking agent; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said cell targeting molecule(s) is indirectly attached to the nanoparticle core via a second functional group on the first crosslinking agent and one or more of said biologically active molecule(s) is indirectly attached to the nanoparticle core via a second functional group on the second crosslinking agent.

Within further embodiments, the present disclosure provides functionalized nanoparticles for promoting the repair of genetic mutations in target nucleic acids, which functionalized nanoparticles include (a) a nanoparticle core; (b) a polymer coating or lipid bilayer that encapsulates the nanoparticle and having first and second functional groups that are associated with and/or attached directly thereto; (c) one or more cell targeting molecule(s), including one or more a cell membrane-penetrating molecules, such as an HIV Tat derived peptide or other peptide having, for example, from five to nine basic amino acids, including arginine and/or lysine; and (d) one or more biologically active molecule(s) wherein one or more of said biologically active molecule(s) is selected from the group consisting of(i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and wherein one or more of said cell targeting molecule(s) is attached directly to the polymer coating or lipid bilayer via a first functional group on the polymer coating or lipid bilayer and one or more of said biologically active molecule(s) is attached directly to the polymer coating or lipid bilayer via a second functional group on the polymer coating or lipid bilayer.

Within still further embodiments, the present disclosure provides functionalized nanoparticles for promoting the repair of genetic mutations in target nucleic acids, which functionalized nanoparticles include (a) a nanoparticle core; (b) a polymer coating or lipid bilayer that encapsulates the nanoparticle and having first and second functional groups that are associated with and/or attached directly thereto (c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent; (d) one or more cell targeting molecule(s), including one or more a cell membrane-penetrating molecules, such as an HIV Tat derived peptide or other peptide having, for example, from five to nine basic amino acids, including arginine and/or lysine; and (e) one or more biologically active molecule(s) wherein one or more of said biologically active molecule(s) is selected from the group consisting of(i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid; wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

Suitable nanoparticle cores include metal nanoparticle cores, such as an iron or gold containing nanoparticle cores, synthetic nanoparticle cores, and ceramic nanoparticle cores having hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

Within certain embodiments, the present disclosure provides functionalized nanoparticles that can be used in methods for the regulation of gene expression. Exemplified herein are gold functionalized nanoparticles in which one or more of the biologically active molecule(s) are small inhibitory RNAs (siRNAs).

Engineered siRNA-modified nanoparticles offer a delivery system useful for blocking gene function and for sequence-specific post-transcriptional gene silencing. siRNA-modified nanocarriers can enter cells, where siRNA strands unwind and assemble into an effector RNA Induced Silencing Complex (RISC). The catalytic RISC recognizes mRNAs containing perfect or near-perfect complementary sequence to the guide siRNA antisense strand. The antisense strand then binds to its complementary/target mRNA (activated RISC/mRNA complex). The catalytic RISC recognizes mRNAs containing perfect or near-perfect complementary sequence to the guide siRNA and cleaves the mRNAs at a site precisely 10 nucleotides from the 5′-end of the guide strand. Finally, mRNA degradation is achieved by endo- and exonucleases, resulting in knockdown of the expression of the corresponding genes, thus mediating translational repression or induction of chromatin modification.

Recently, Conde et al. provided evidence of in vitro and in vivo RNAi triggering via synthesis of a library of novel multifunctional gold nanoparticles, using a hierarchical approach including three biological systems of increasing complexity: in vitro cultured human cells, in vivo freshwater polyp (Hydra vulgaris), and in vivo mice models. The authors developed effective strategies to conjugatespecific biomolecules to the surface of gold nanoparticles in a highly controlled way. These biomolecules included (a) biofunctional spacers (Poly(ethylene glycol) (PEG) spacers used to increase solubility and biocompatibility); (b) cell penetrating peptides such as TAT and RGD peptides, which exploit more than one mechanism of endocytosis to overcome the lipophilic barrier of the cellular membranes and deliver bioactive molecules of varying size inside the cell; and (c) siRNAs complementary to a master regulator gene, the protooncogene c-myc, which were bound covalently (thiol-siRNA) and ionically (naked/unmodified siRNA) to gold nanoparticles.

### E. Methods for Using Functionalized Nanoparticles

The present disclosure provides functionalized nanoparticles that may be advantageously employed in (1) methods for the treatment of diseases and disorders, in particular human diseases and disorders; (2) methods for inducing the reprogramming, including direct reprogramming, of mammalian cells, including somatic cells and stem cells; (3) methods for promoting the repair of target nucleic acids; and (4) methods for repairing mutant genes and gene editing.

Thus, within certain embodiments, provided herein are methods for using the presently disclosed functionalized nanoparticles, which methods include contacting a cell with a functionalized nanoparticle that comprises (1) a biologically active molecule for effectuating (i.e., regulating, modulating, normalizing, and/or restoring) one or more functions of the cell such as, for example, maintenance, survival, growth/proliferation, differentiation, and/or death and (2) a targeting molecule, such as a cell membrane-penetrating molecule for binding to and penetrating a membrane of the cell, including a plasma membrane, a nuclear membrane, a mitochondrion membrane, an endosomal membrane, a lysosomal membrane, and/or other membrane, thereby facilitating the delivery of the functionalized nanoparticle to the cell and effectuating the one or more cellular functions by the biologically active molecule.

Within other embodiments, provided herein are methods for using the presently disclosed functionalized nanoparticles, which methods include administering to a patient having a disease or disorder a functionalized nanoparticle that comprises (1) a biologically active molecule for effectuating (i.e., regulating, modulating, normalizing, and/or restoring) one or more functions of a cell within the patient such as, for example, maintenance, survival, growth/proliferation, differentiation, and/or death and (2) a targeting molecule, such as a cell membrane-penetrating molecule for binding to and penetrating a membrane of a cell of the patient having a disease or disorder, including a plasma membrane, a nuclear membrane, a mitochondrion membrane, an endosomal membrane, a lysosomal membrane, and/or other membrane, thereby facilitating the delivery of the functionalized nanoparticle to the cell and effectuating the one or more cellular functions by the biologically active molecule thereby alleviating one or more aspects of the disease or disorder.

The methods disclosed herein utilize functionalized nanoparticles including, for example, superparamagnetic iron oxide particles similar to those previously described in scientific literature. This type of nanoparticle can be used in clinical settings for magnetic resonance imaging of bone marrow cells, lymph nodes, spleen and liver. See, e.g., Shen et al., Magn. Reson. Med. 29:599 (1993); Harisinghani et al., Am. J. Roentgenoi. 172:1347 (1999). These magnetic iron oxide nanoparticles contain ˜5 nm nucleus coated with cross-linked dextran and having ˜45 nm overall particle size.

Importantly, it has been demonstrated that these nanoparticles, further containing cross-linked cell membrane-permeable Tat-derived peptide, efficiently internalize into hematopoietic and neural progenitor cells in quantities of up to 30 pg of superparamagnetic iron nanoparticles per cell. Lewin et al., Nat. Biotechnol. 18:410 (2000). Furthermore, the nanoparticle incorporation does not affect proliferative and differentiation characteristics of bone marrow-derived CD34+primitive progenitor cells or the cell viability. Id. These nanoparticles can be used for in vivo tracking the labeled cells.

The labeled cells retain their differentiation capabilities and can also be detected in tissue samples using magnetic resonance imaging. Here we present novel nanoparticle-based devices which are now functionalized to carry peptides and proteins that can serve as excellent vehicles for intracellular delivery of biologically active molecules for cell reprogramming solutions to target intracellular events and modulate cellular function and properties.

In another embodiment, the present disclosure provides methods for delivering bioactive molecules to a mammalian cell and/or for modulating cellular functions by contacting a mammalian cell with a functionalized nanoparticle as described herein.

For example, mammalian cells, such as fibroblasts or other suitable cell types, which are either commercially available or are obtained using standard or modified experimental procedures, can be plated under sterile conditions on a solid surface, with or without a substrate to which the cells adhere (e.g., feeder cells, gelatin, martigel, laminin, fibronectin, etc.).

The plated cells can be cultured for such a time and in the presence of such factors that allows cell division/proliferation, maintenance, and or cell viability. Examples of such factors include serum and/or various growth factors/cytokines, which can later be withdrawn or refreshed and the cultures continued. The plated cells can be cultured in the presence of functionalized nanoparticles, as described herein, with one or more bioactive molecules attached using the various methods of the present disclosure.

In the case of functionalized nanoparticles that are made from a superparamagnetic material, such as iron oxide, a magnetic field may be advantageously employed to increase the contact surface area between one or more mammalian cells and one or more nanoparticles, which thereby provides improved penetration of functionalized nanoparticles through the cell membrane. A cell population can, as appropriate, be repeatedly treated with functionalized nanoparticles to enhance the intracellular delivery of the associated bioactive molecules.

Cells can be suspended in culture medium and non-incorporated nanoparticles can be removed by centrifugation or by cell separation, leaving cells that are present as clusters. The clustered cells can then be resuspended and recultured in fresh medium for a suitable period. The cells can be taken through multiple cycles of separating, resuspending, and reculturing, until a consequent biological effect triggered by the intracellularly-delivered specific bioactive molecules is observed.

Within other aspects, the present disclosure provides methods for screening for one or more compounds that are effective in achieving the reprogramming of a cell. Such methods involve attaching a test compound to a nanoparticle using one or more of the methods disclosed herein with a cell population of interest, culturing for a suitable period of time, and determining a modulatory effect resulting from the test compound. Such modulatory effects can include, for example, initiation of cell reprogramming; generation of pluripotent stem cells; differentiation or trans-differentiation of cells to more-specialized or differently-specialized cell types; examination of cells for toxicity, metabolic change, or an effect on contractile activity and other functions.

Within further aspects, the present disclosure provides methods for preparing specialized cells as a medicament and/or delivery device for the treatment of a human or other animal in need thereof. This enables the clinician to administer the cells in or around the damaged tissue (whether heart, muscle, liver, etc.) either from the vasculature or directly into the muscle or organ wall, thereby allowing the specialized cells to engraft, limit the damage, and participate in regrowth of the tissue's musculature and restoration of specialized function.

Still another use of the present disclosure is the formulation of specialized cells as a medicament or in a delivery device intended for treatment of a human or animal body. This enables the clinician to administer the cells in or around the damaged tissue (whether heart, muscle, liver, etc) either from the vasculature or directly into the muscle or organ wall, thereby allowing the specialized cells to engraft, limit the damage, and participate in regrowth of the tissue's musculature and restoration of specialized function.

E1. Methods for Reprogramming a Cell

One exemplary application of the presently-disclosed functionalized nanoparticles is in methods for reprogramming a mammalian cell, such as a fibroblast or other somatic cell, into a stem cell or another cell type, which methods comprise contacting a mammalian cell, such as a mammalian fibroblast cell or other somatic cell, with a functionalized nanoparticle that includes as constituent bioactive molecules the transcription factors Oct4 and Sox2.

The present disclosure provides methods for promoting the differentiation of a cell into an induced cardiomyocyte-like cell (iCM), which methods include contacting the cell with a functionalized nanoparticle comprising a nanoparticle core, including a metal nanoparticle core, such as an iron or gold containing nanoparticle core, a synthetic nanoparticle core, or a ceramic nanoparticle core, to which a cardiomyocyte inducing agent and a cell targeting molecule is attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Gata-4, Mef2C, Tbx5, Mesp1, Hand2, MyoCD, Mir-1, Mir-133, CHIR99021, A83-01, BIX01294, AS8351, SC1, Y27632, OAC2, Y27632, OAC2, SU16F, JNJ10198409, Oct4, Sox2, Klf4, and o-Myc, or a functional domain or structural variant thereof.

The present disclosure also provides methods for promoting the differentiation of a cell into an induced pluripotent stem cell (iPSC), which methods include contacting the cell with a functionalized nanoparticle comprising a nanoparticle core, including a metal nanoparticle core, such as an iron or gold containing nanoparticle core, a synthetic nanoparticle core, or a ceramic nanoparticle core, to which a stem cell inducing agent and a cell targeting molecule is attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable cardiomyocyte inducing agents include Lin28, Nanog, Mir-302bcad/367, Mir-302, Mir-200c, Mir-369, Oct4, Sox2, Klf4,and c-Myc, or a functional domain or structural variant thereof.

The present disclosure also provides methods for promoting the differentiation of a cell into an induced neuronal cell (iNC), which methods include contacting a cell with a functionalized nanoparticle comprising a nanoparticle core, including a metal nanoparticle core, such as an iron or gold containing nanoparticle core, a synthetic nanoparticle core, or a ceramic nanoparticle core, to which a neuronal cell inducing agent and a cell targeting molecule are attached. Suitable nanoparticle cores have hydrodynamic diameters of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm. Suitable neuronal cell inducing agents include Brn2, Asc11, Myt11, Zic1, Mir-9, Mir-124, NeuroD1, Lmx1a, FoxA2, Oct4, Sox2, Klf4, c-Myc, or a functional domain or structural variant thereof.

A related application of the presently-disclosed functionalized nanoparticles regards screening one or more test compounds for the reprogramming of a mammalian cell, such as a fibroblast, into a stem cell or another cell type, or for effect on cell reprogramming. These methods comprise contacting a mammalian cell with a functionalized nanoparticle that includes as a constitutent bioactive molecule the one or more test compounds, culturing for a suitable period of time, and determining a modulatory effect resulting from the one or more test compounds. This may include initiation of the cell reprogramming and generation of pluripotent stem cells, differentiation or trans-differentiation of cells to more specialized or differently-specialized cell types, examination of the cells for toxicity, metabolic change, or an effect on contractile activity and other functions.

In another aspect, the present disclosure is also directed to a method of delivering bioactive molecules attached to functionalized nanoparticles for modulation of intracellular activity aimed at direct reprogramming of human somatic cells into iCM. For example, human cells, fibroblasts or other cell types that are either commercially available or obtained using standard or modified experimental procedures are first plated under sterile conditions on a solid surface with or without a substrate to which the cells adhere (feeder cells, gelatin, martigel, fibronectin, and the like). The plated cells are cultured for a time with a specific factor combination that allows cell division/proliferation or maintenance of acceptable cell viability. Examples are serum and/or various growth factors as appropriate for the cell-type, which can later be withdrawn or refreshed and the cultures continued. The plated cells are cultured in the presence of functionalized biocompatible cell-permeable nanoparticles with covalently linked cell-specific reprogramming factors (reprograming factors specific for the cell type of interest, such as for example, cardiac-, hepatocyte, and neural-specific reprogramming factors) attached using various methods briefly described herein and elsewhere (see, e.g., US 2014/0342004, incorporated herein by reference in its entirety) in the presence or absence of magnetic field. The use of a magnet in case of superparamagnetic nanoparticles renders an important increase in the contact surface area between the cells and nanoparticles and thereby reinforces further improved penetration of functionalized nanoparticles through the cell membrane. When necessary, the cell population is treated repeatedly with the functionalized nanoparticles to deliver the bioactive molecules intracellularly.

The cells are maintained attached or suspended in culture medium, and non-incorporated nanoparticles are removed by centrifugation or cell separation, leaving cells that are present as clusters. The cells are then resuspended and recultured in fresh medium for a suitable period. The cells can be taken through multiple cycles of separating, resuspending, and reculturing, until a consequent direct reprogramming effect triggered by the specific bioactive molecules linked to the functionalized nanoparticles is observed. The current disclosure is applicable not only to direct reprogramming of one type of cells into another, but also as new means to control or regulate the cell fate with preservation of the original cell type. A broad range of cell types can be used such as human fibroblasts, blood cells, epithelial cells, mesenchymal cells, and the like.

Cell reprogramming, whether direct or indirect, is based on the treatment of various cell types or tissues with bioactive molecules that can include various proteins, peptides, small molecules, microRNAs, siRNAs, shRNAs, mRNAs, and the like. Such bioactive molecules do not penetrate through cell membrane efficiently, or at all, and may not reach the cell nuclei without a special delivery vehicle. Furthermore, these bioactive molecules have short half-life and can undergo degradation upon exposure to various proteases and nucleases. These disadvantages result in reduced efficacy of the bioactive molecules and require much higher or repeated doses of a treatment to achieve a noticeable cell reprogramming effects, if any. Therefore, in the current disclosure functionalized nanoparticles are used to overcome the abovementioned disadvantages. More specifically, these bioactive molecules when linked to the nanoparticles and compared with the original “naked” state, acquire new physical, chemical, biological functional properties, that confer cell-penetrating and cell nucleus-targeting ability, larger size and altered overall three-dimensional conformation and the acquired capability to regulate the expression of target genes of interest.

To date, a number of gene products and bioactive molecules have been reported to exhibit reprogramming effects, and the list continues to grow. For example, different sets of bioactive molecules and/or gene products were reported to induce direct reprogramming of human fibroblasts to cardiomyocytes. One such set represents a group of transcription factors. Another set includes some of these factors and additional genes along with microRNA molecules miR1 and miR133. Yet other sets include different combinations of bioactive molecules as reported (Fu et al., Stem Cell Reports 1:235-247 (2013); Nam et al., Proc. Natl. Acad. Sci. USA. 110:5588-5593 (2013); Wada et al., Proc. Natl. Acad. Sci. USA. 110:12667-12672 (2013); and Cao et al., Science 352(6290):1216-1220 (2016)).

Human fibroblasts transduced with viruses harboring these bioactive molecules have been reprogrammed directly into induced cardiomyocyte-like cells (iCM) as evidenced by presence of cardiac-specific markers absent in original fibroblasts. Yet, the resultant reprogrammed cells have a skewed gene expression pattern that is due to insertion of the viral and gene product-encoding DNA into the cell genome. Furthermore, the efficiency of such direct reprogramming is very low, which in part is due to a short half-life of these bioactive molecules. These problems are addressed by the present disclosure, which provides for the use of additional degradation-protecting compounds, such as a nanoparticle or a PEG or other compound or molecule functionalized with non-integrating peptides, proteins and RNA molecules, thereby preserving the cell genome intact.

In addition to direct fibroblast-to-cardiomyocyte reprogramming, direct reprogramming have been reported possible for the generation of hepatocytes and neural cells using different sets of bioactive molecules. For example, the FOXA3, HNF1A, and HNF4A genes, when expressed in human fibroblasts using lentiviral vectors, result in direct reprogramming of the cells and generation of functional hepatocytes as evidenced by the expression of hepatic genes and restoration of liver function in an animal model of acute liver failure. Similar to the virus-mediated direct cardiac reprogramming, this approach may result in detrimental consequences due to random integration of viral DNA into the human cell genome and development of cancer. The present disclosure overcomes this problem upon generation and use of the nanoparticles functionalized using abovementioned and/or other reprogramming factors as non-integrating molecules thereby preserving the cell genome completely intact.

Successful fibroblast-to-neural cell direct reprogramming was reported upon treatment of fetal fibroblasts with a single factor, Sox-2. See, Ring et al., Cell Stem Cell. 11:100-109, (2012); incorporated herein by reference in its entirety). The resultant newly reprogrammed cells exhibit neural cell phenotype and gene expression pattern with the ability to further differentiation to other neural cell types such as oligodendrocytes and astrocytes. More recently expression of Sox2 and Pax6 genes was reported to be effective in reprogramming human adult fibroblasts into neural cells (Connor et al., Protocol Exchange (2015), doi: 10.1038/protex.2015.034; incorporated herein by reference in its entirety). There are various factors or their combinations that reprogrammed human somatic cells such as fibroblasts directly to neural cells. See, e.g., Son et al., Cell Stem Cell 9:205-218 (2011); Pfisterer et al., Proc. Natl. Acad. Sci. 108:10343-10348 (2011); and Ambasudhan et al., Cell Stem Cell 9:113-118(2011).

Similar to other reports on transdifferentiation, the direct reprogramming approaches indicated above are also based on the expression of gene products delivered to the cells using either lentiviral or retroviral vectors or plasmid DNA. Again, the use of DNA is prone to trigger unpredictable random insertion of nucleotides into the genomic DNA of the host cell thereby potentially leading to detrimental consequences or skewing the phenotype. However, attempts to implement cell reprogramming using reprogramming factors such as proteins fused to TAT-like peptides with cell-penetrating ability for cell reprogramming has been very inefficient compared with viral delivery of the genes of interests. See, Kim et al., Cell Stem Cell 4:472-476 (2009) and Zhou et al., Cell Stem Cell 4:381-384 (2009), which is the major reason this approach was abandoned and not followed.

The present disclosure overcomes the insertional mutagenesis and skewing genotype/phenotype problems by using nanoparticles (whether metal-core (e.g., superparamagnetic iron-based or gold based nanoparticles) or non-cored (e.g., polymeric nanoparticles)) functionalized with any of the abovementioned or other bioactive molecules exposure to which may result in reprogramming of one type of cells into another cell type. The recited cell types, factors, and/or combinations of factors are not intended to be limiting and that additional factors and/or combinations will be newly discovered and that those combinations would work in the same way as described in the application.

One use of the functionalized nanoparticles disclosed herein is the screening/testing of a biologically active molecules for an effect on cell reprogramming. This involves combining the compound attached to the nanoparticle using methods disclosed herein with a cell population of interest (whether fibroblasts, blood cells, mesenchymal cells, and the like), culturing for suitable period and then determining any modulatory effect resulting from the compound(s). This includes direct cell reprogramming and generation of specialized cell types of interest, such as cardiac cells, hepatocytes (liver cells), or neural cells, examination of the cells for toxicity, metabolic change, or an effect on contractile activity and/or other function.

Within other embodiments, the present disclosure provides methods for the direct reprogramming of a somatic cell, such as a fibroblast or other differentiated somatic cell, into a functional cell having a selected (predetermined) lineage such as a cardiac cell, a hepatocyte, and a neural cell. Within other aspects of these embodiments, the present disclosure provides methods for the direct reprogramming of a somatic cell, such as a fibroblast or other differentiated somatic cell, into a stem cell, such as an induced pluripotent stem cell (iPSC) or other undifferentiated cell type.

Within certain aspects of these embodiments are provided methods for the direct reprogramming of a somatic cell, such as a fibroblast or other differentiated somatic cell, that is obtained from a human subject that is afflicted with a neurodegenerative disease or disorder or at risk for developing a neurodegenerative disease or disorder. As disclosed herein, somatic cells may, for example, be obtained from a human subject that is afflicted with a neurodegenerative disease or disorder that is selected from leukencephalopathy, leukodystrophy, adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g., stroke, head injury, cerebral palsy) or is a condition characterized by apraxia, achalasia, or epilepsy.

In one embodiment, the condition being treated is ALSP. ALSP is associated with mutations in the tyrosine kinase domain of the CSF-1R gene (1, 4, 17-20) (also referred to as CD115, FIM2, FMS, or M-CSF-R) which encodes for a 107.984 KD transmembrane G-Protein coupled receptor that is a member of the CSF1/PDGF receptor family. The 60.002 Kb gene resides on chromosome 5q32 and codes for the 972 amino acid protein. CSF-1R is a class III receptor tyrosine kinase expressed on myeloid cells, particularly microglia and differentiated macrophages, but has also been detected on Paneth cells, osteoclasts, renal tubular cells, oocytes and colonic epithelial cells (21). Evidence for its expression on neuronal lineage cells has also been detected (22).

Over 45 mutations for the disease have been identified in the coding region for the kinase portion of the CSF-R1 protein all leading to loss of function due to defective phosphorylation of receptor sites and downstream effectors. Mutations associated with the ALSP have been found on exons 13, 14, 15, 17, 18, 19, 20 and 21 of the CSF-1R gene that has a total of 22 exons. CSF-1R is a receptor for 2 ligands, CSF-1 and interleukin-34 (IL-34) with distinct in vivo spatiotemporal patterns of expression. Although CSF-1R null mice have a severe phenotype, a recently developed haploinsufficient mouse model produced an adult phenotype of early onset dementia resembling the human ALSP phenotype. Even in leukencephalopathies where the primary lesion is not thought to be microglia, positive outcomes in both murine models and clinical trials with allogeneic or genetically corrected bone marrow stem cell transplant have been attributed to the penetration of transplantation-derived monocytes and macrophages into the brain and subsequent differentiation into microglia in both murine models and human clinical trials.

Within certain embodiments, functionalized nanoparticles disclosed herein may be used in methods for the differentiation of corrected cells to microglia. There are different paths to generate microglia-like cells from niPSCs that can incorporate elements of previously published methods, the publications of which are incorporated herein by reference in their entireties. Sievers and coworkers established in 1994 that monocytes and macrophages grown on monolayers of astrocytes differentiate into microglia-like cells. Microglia have been successfully differentiated from mouse ESCs and from human monocytes. In one embodiment, genetically corrected human niPSCs from ALSP patients can be differentiated to microglia-like cells through nestin+ neuronal precursors or through monocytes.

In one embodiment, niPSCs can be differentiated to microglia-like cells through nestin+ progenitors following inhibition of SMAD. While many methods for differentiating stem cells to neural progenitors are via embryoid bodies (EB), SMAD inhibition bypasses this step and eliminates the need for manual clonal selection. A kit is now available (Applied Stem Cell; Milpitas, Calif.) for this procedure.

In another embodiment niPSCs with naïve mouse ESC-like morphology effectively form a monolayer of single cells as well as relatively uniform EBs in suspension cultures in the absence of anti-differentiation agents like 2iL (inhibitors of ERK1/2 and GSK3β signaling and human Leukemia Inhibitory Factor). At 4-8 d after EB body formation, visibly distinguishable EBs bodies can be selected and replated on fibronectin-coated tissue culture dishes. After 2 d, the medium can be switched to B27-medium with rhFGF2 and 5 ug/ml fibronectin and cultured for 14 days, changing media every 2 days. In one embodiment, after 14 days Nestin+ cells are expanded in N2-medium with rhFGF2 and 10 ng/ml laminin.

Differentiation of neural progenitors directly from iPSCs obviates the need for embyoid body formation and attendant variability. It is well known to those skilled in the art that dual SMAD inhibition, for instance with Noggin and SB431542, is sufficient to promote neural conversion from human ESCs and iPSCs (43).

The first step in differentiation is to make a single cell suspension of niPSCs. One method familiar to those skilled in the art could include rinsing with 1× Ca²⁺/Mg²⁺ free Dulbecco's Phosphate-Buffered Saline or similar buffer and then treating with an enzyme such as Accutase for 5˜7 minutes at 37° C. Cells can then be incubated with 250 ng/ml noggin (such as available from R&D Systems) and 10 μM SB431542 (such as available from R&D Systems) in appropriate tissue culture plates. Every day for 5 days, 50˜75% of the media should be replaced with fresh media supplemented with 250 ng/ml noggin and 10 μM SB431532. On the fifth day of differentiation, cell clusters can be collected and re-plated in 6-well plates coated with Matrigel™ in media supplemented with 250 ng/ml noggin and 10 μM SB431532. Cells cultured in this way with media replacement on an every other day basis for an additional four to five days should result in the formation of neural rosettes composed of neural progenitors that can be harvested and tested for Nestin expression. Nestin+ neural progenitors can be administered as described herein to subjects in need thereof or further differentiated to Microglia-like cells.

Microglial differentiation: To initiate microglial differentiation, the medium will be switched to N2 medium supplemented with fibroblast growth factor-2 (FGF2) and laminin to enhance microglial differentiation and expansion. In the final step of the differentiation (day 26), growth factors will be removed. Stably proliferating niPSC-derived microglia should be visible by 3 weeks. To purify the microglia, single cell colonies can be selected manually and cultured in serum-free N2 medium. The cells will be split using a cell scraper at 80% confluency.

Differentiation of hPSCs into myeloid cells is well known to those skilled in the art. A detailed protocol was published in 2011 (44) and techniques for optimizing monocyte generation using both chemically defined and semi-defined protocols have been subsequently established and well known to those skilled in the art (45, 46). In one embodiment, monocytic cells can be generated from EBs produced from niPSCs as described herein. EBs can be cultured in X-VIVO™ 15 (such as available from Lonza) supplemented with 100 ng/mL M-CSF, 25 ng/mL IL-3, 2 mM glutamax, 100 U/mL penicillin, 100 mg/mL streptomycin, and 0.055 mM b-mercaptoethanol. From 2-3 weeks, monocytes should be visible in the supernatant of the cultures and can be harvested for characterization and differentiation to microglia as described below. One skilled in the art can characterize niPSC-derived monocytes morphologically and by antibody binding to cell surface markers CD14, CD16, CD163, CD86, CD38, CD34, MHCII and CD45 via flow cytometry. As an example, expression of the LPS receptor, CD14 is expected to be high vs. CD16 low.

In one embodiment of the method monocytes isolated from the blood of human donors and niPSC-derived monocytes produced as described in this document can be cultured in RPMI-1640 Glutamax (Invitrogen) supplemented with 100 U/mL penicillin, 100 mg/mL streptomycin and a mixture of the following cytokines; recombinant human GM-CSF (10 ng/ml), recombinant human IL-34 (100 ng/ml) and M-CSF (10 ng/ml) for 14 days to generate microglia-like cells.

In one aspect of the method, differentiation can be confirmed by co-expression of characteristic markers. For example, one skilled in the art can use real time PCR or antibodies to key proteins to establish differentiation. In a further aspect of this method, differentiation to microglia-like cells can be confirmed by co-expression of characteristic markers and compared with monocytes and nestin+ neural precursors. While multiple studies document murine microglia marker expression, human primary microglia expression analysis has recently identified unique markers (47, 48). Taking these findings into consideration, one can assess the following markers: One skilled in the art can assess protein expression by flow cytometry with antibodies to the following exemplary cell surface proteins: CD11b, CD11c, CD29, CD36, CD45, CD80, CD86, TREM2, and CD115 (also referred to as CSF1R). Expression of stem cell proteins CD34 and CD117 can also be assessed and should be only weakly detectable or unexpressed in differentiated cells. One can also assess CD115 expression with mRNA analysis. Expression of PROS1, GAS6, MERTK, GPR34 and P2Ry12 can be assessed by qPCR.

It will also be possible for someone skilled in the art to quantify phagocytosis. For instance, a quantitative assay of phagocytosis could employ CytoSelect 24-Well Phagocytosis Assay kit (zymosan, colorimetric format, such as available from Cell Biolabs, San Diego, Calif.) as described (49). Phagocytosis can be assessed in microglia at 60-70% confluency in 24 or 96 well plates in samples and negative controls without zymosan in triplicates or quadruplicates. In one aspect, zymosan particles can be resuspended in PBS at 5×10⁸ particles/ml from which 20 μl suspension of non-opsonized zymosan particles can be added per well of 24-well plate and incubated for 1 hr at 37° C. External zymosan particles can be blocked following fixation, followed by permeabilization and colorimetric detection of engulfed particles at 405 nm.

Cytokine responses to lipopolysaccharide (LPS) is an indication of cytokine production. To measure this, cells can be stimulated with LPS (10, 20, 40 ng/m) for 2, 4 and 6 hours as described (48) and cytokine and marker expression measured by qRT-PCR. In one aspect, TNFalpha and IL6 can be measured along with markers recently shown to be distinctive of primary human microglia vs. microglial cell line (BV2): CX3CL1, IFNB1, STAT1.

Cell migration can be evaluated through several different methods including scratch assays, cell-exclusion zone assays, microfluidic based assays, and Boyden Chamber assays. The most widely accepted cell migration technique is the Boyden Chamber assay. The classic Boyden Chamber system uses a hollow plastic chamber, sealed at one end with a porous membrane. This chamber is suspended over a larger well which may contain medium and/or chemoattractants. Cells are placed inside the chamber and allowed to migrate through the pores, which can be various sizes, to the other side of the membrane. Migratory cells are then stained and counted. Chemotaxis of monocytic cells and microglia can be measured by one skilled in the art using Boyden chambers preferably using a pore size of 5 micrometers. One property of microglia is directed migration toward a gradient of CX3CL1 (20 ng/ml). Microglia cell migration should be at least 50% higher than in cells without CXCL1 receptors.

Whole genome sequencing can be performed to confirm the preservation of the genome integrity after reprogramming with non-integrating functionalized nanoparticles and CRISPR gene editing. For instance, in one embodiment the following samples could be evaluated: the original fibroblasts generated from skin tissues of the ALSP patients and healthy donors that were used for reprogramming and stored in liquid nitrogen, the niPSCs trypsinized and cultured on matrigel or laminin for ˜15-20 passages, the original fibroblasts also cultured for 15-20 passages and the newly generated microglia and microglia progenitors. Thus, 4 or more different cell subpopulations generated throughout each reprogramming could be evaluated. In one embodiment, the genomic DNA can be obtained from the samples using Qiagen Genomic DNA Isolation kit. Isolated DNA can be evaluated using a number of different techniques by one skilled in the art. For example, samples can be evaluated by whole genome sequencing, whole exome sequencing or targeted Array. The data generated by these assays can be used to validate the preserved genome integrity. Karyotype of reprogrammed cells can also be evaluated using G-banding technique. These data will validate preservation of normal karyotype and genome integrity of reprogrammed and differentiated cells.

E2. Methods for the Treatment of Diseases and Disorders

Within several embodiments, the present disclosure provides methods for the treatment of diseases and disorders, which methods comprise the administration of a functionalized nanoparticle as described in detail herein.

The present disclosure provides methods for the treatment of a neurodegenerative disease or disorder in a patient, which methods include administering to the patient a functionalized nanoparticle comprising a nanoparticle core to which a stem cell inducing agent and a cell targeting molecule are attached. The present disclosure also provides methods for the treatment of a neurodegenerative disease or disorder in a patient, which methods include administering to the patient an iPSC produced by methods and using functionalized nanoparticles as disclosed herein. The present disclosure also provides methods for the treatment of a neurodegenerative disease or disorder in a patient, which methods include administering to the patient a functionalized nanoparticle comprising a nanoparticle core to which a neuronal cell inducing agent and a cell targeting molecule are attached.

The present disclosure also provides methods for the treatment of a neurodegenerative disease or disorder in a patient, which methods include administering to the patient a functionalized nanoparticle comprising a nanoparticle core to which is attached (a) one or more biologically active molecule(s) such as (i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including one or more a cell membrane-penetrating molecules, such as an HIV Tat derived peptide or other peptide having, for example, from five to nine basic amino acids, including arginine and/or lysine.

Neurodegenerative diseases or disorders that may be treated according to these methods include, for example, leukencephalopathy, leukodystrophy, Adult onset leukencephalopathy with spheroids and pigmented Glia (ALSP), Multiple Sclerosis, periventricular leukomalacia, Parkinson's Disease, Alzheimer's Disease, Epilepsy, Depression, Lewy-body Dementia, Amyotropic Lateral Sclerosis, vasculitis, pigmented orthochromatic leukodystrophy (POLD), Schizophrenia, Bipolar Disorder, Autism, idiopathic leukencephalopathy, Niemann Pick Disease, Nasu-Hakola Disease, Rett Disease, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g. stroke, head injury, cerebral palsy) or is a condition characterized by apraxia, achalasia, and epilepsy.

Within certain aspects of these embodiments, the neurodegenerative disease or disorder is Nasu-Hakola disease and the target nucleic acid is the CSF-1R gene. Within other aspects, the neurodegenerative disease or disorder is Nasu-Hakola disease and the target nucleic acid is the TREM2 gene. Within further aspects, the neurodegenerative disease or disorder is metachromatic leukodystrophy (MLD) disease and the target nucleic acid is the Arylsulfatase A gene. In related embodiments, the present disclosure provides methods for treating a neurodegenerative diseases and disorders in a subject that is afflicted with a neurodegenerative disease or disorder or at risk for developing a neurodegenerative disease or disorder. Such methods comprise administering specialized cell types that are generated from one or more cells that are sourced from the subject and that include, without limitation, induced pluripotent stem cells that are induced from somatic cells that are contacted with a functionalized nanoparticle as disclosed herein.

In certain aspects of these embodiments, methods employ non-integrating functionalized nanoparticles to reprogram somatic cells from a patient afflicted with or at risk for developing a neurodegenerative diseases or disorder, wherein the somatic cells are reprogramed into pluripotent stem cells that are contacted with a functionalized nanoparticle as disclosed herein.

In other aspects of these embodiments, the induced pluripotent stem cells are made from somatic cells from an individual with a genetic leukodystrophy wherein the gene has been corrected prior to administration to the subject. Gene correction may employ a CRISPR/Cas9 gene editing system or other gene editing approaches available in the art such as, for example, CRISPR nanoparticle conjugates as described in U.S. Patent Application No. 62/406,542, incorporated herein by reference in its entirety, and/or as described elsewhere herein. The source somatic cells can be any nucleated cell from the patient, such as skin or blood cells, including monocytes.

The gene that is corrected may be the CSF-1R gene, particularly in the region of the gene coding for the tyrosine kinase function. Alternatively, the gene that is corrected may be TREM2 or ARSA. The gene that is corrected may be related to a patient's hematopoietic disorder, such as a hematopoietic disorder that may evolve into a leukemia. In such cases, the target gene for correction can include ELANE (a neutrophil elastase gene), HAX-1, WAS, or one or more other gene(s) that, when mutated, contribute to a hematopoietic disorder.

In further aspects of these embodiments, induced pluripotent stem cells may be derived from blood cells or skin cells from a patient with ALSP that has a mutation in the CSF-1R gene that is subsequently corrected by CRISPR and/or nanoparticle conjugates. Corrected induced pluripotent stem cells may be expanded ex vivo and differentiated into macrophages, microglia, or neural progenitors prior to administration to the patient in sufficient quantity to reduce or ameliorate disease symptoms, and/or to restore normal function in vivo. It will be appreciated that the order of pluripotency and gene editing steps can be altered to reach similar results. Thus, a patient's cells can alternatively be used first to correct a mutant gene with subsequent reprogramming into the appropriate state, such as nanoparticle-induced pluripotent stem or other more specialized cell type as needed.

Induced pluripotent stem cells may be differentiated into either macrophages, microglia or neural progenitors prior to genetic correction and used in the presently disclosed methods for treating or preventing a neurodegenerative disease in a subject by administering to the subject an effective amount of genetically corrected induced pluripotent stem cell-derived cells including, but not limited to, macrophages, microglia, or neural progenitor cells. Within certain aspects, these methods optionally include selecting a subject with a neurodegenerative disease of the central nervous system (for example, at an early stage of disease) or at risk for a neurodegenerative disease of the central nervous system.

Within certain methods, an effective amount of corrected induced pluripotent stem cells are administered to a patient to diminish or ameliorate a disease state and/or to restore normal function in neural cells, ameliorate symptoms of neural degeneration, and/or prevent or inhibit the onset of symptoms in a patient predisposed to a neuronal disease or disorder by way of genetic mutations. The method optionally includes the determination and/or confirmation that a subject has a mutation in the CSF-1R gene, the TREM2 gene, and/or the ARSA gene as compared to normal control subjects. Such a determination can be performed before the generation of gene-corrected induced pluripotent stem cells.

Within certain aspects of these methods, the patient may be diagnosed as having, suspected of having, or at risk of having ALSP or Multiple Sclerosis, Parkinson's Disease, periventricular leukomalacia, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD, Rett Disease, apraxia, achalasia, epilepsy, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelinosis, spongiform leukodystrophy, and leukodystrophy (Alexander type); and acute brain injury (e.g., stroke, head injury, cerebral palsy, and the like).

Neurological diseases and disorders that are treatable using the methods disclosed herein include, but are not limited to, Adult onset leukencephalopathy with spheroids and pigmented Glia (ALSP), Multiple Sclerosis, Parkinson's Disease, periventricular leukomalacia, Alzheimer's Disease, Epilepsy, Depression, Lewy-body Dementia, Amyotropic Lateral Sclerosis, vasculitis, POLD, Schizophrenia, Bipolar Disorder, Autism, idiopathic leukencephalopathy, Niemann Pick Disease, Nasu-Hakola Disease, metachromatic leukodystrophy (MLD), Rett Disease, or is a condition characterized by apraxia, achalasia, epilepsy, ischemia, cerebellar ataxia, demyelinating diseases, including disseminated perivenous encephalomyelitis, neuromyelitis optica, concentric sclerosis, acute disseminated encephalomyelitides, post encephalomyelitis, postvaccinal encephalomyelitis, acute hemorrhagic leukoencephalopathy, progressive multifocal leukoencephalopathy, idiopathic polyneuritis, diphtheric neuropathy, Pelizaeus-Merzbacher disease, neuromyelitis optica, diffuse cerebral sclerosis, central pontine myelolinosis, spongiform leukodystrophy, and leukodystrophy (Alexander type); and acute brain injury (e.g., stroke, head injury, cerebral palsy).

Acquired or inherited hematopoietic diseases and disorders that are treatable using the methods disclosed herein include cyclic neutropenia, myelokathexis, severe congenital neutropenia, acute myeloid leukemia, and lymphoblastic leukemias that are due to mutations in corresponding genes.

Another use of the disclosure is the formulation of specialized cells as a medicament or in a delivery device intended for treatment of a human or animal body. This enables the clinician to administer the functionalized nanoparticles in or around the damaged organ (e.g., heart, brain, or liver) tissue either from the vasculature or directly into the muscle or organ tissue, thereby allowing the specialized cells to engraft, limit the damage, and participate in regeneration/regrowth of the tissue's musculature and restoration of specialized function. Alternatively, the induced cardiac cells (iCM) or other cell types, as described herein, can be produced ex vivo with the described functionalized nanoparticles and administered thereafter into the area around diseased or damaged tissue of a subject.

Another application of the present disclosure is to generate and/or use the iCMs as described herein as a screening scaffold to test one or more candidate compositions for a therapeutic or pharmacological effect in a cardiac disease context. For example, the iCMs (or hepatocytes, neural cells, or other cell types of interest) can be generated and cultured in vitro and contacted with a candidate pharmaceutical agent and the cells can thereafter be observed for an effect. In some embodiments, an iCM or other cell type, can be generated from a somatic cell derived from a subject with a cardiac disorder or other diseases. Accordingly, the screen for pharmaceutical activity with respect to the cardiac condition can be made for the specific genetic background of the subject in need to assess the responsiveness of the subject to the pharmaceutical agent.

Methods of Delivery of niPSC products, such as gene-corrected microglia, macrophages or neural progenitors, to the brain for the treatment of ALSP, Nasu-Hakola, MLD or other leukenecephalopathies, or other neurodegenerative disorders or conditions, are now described.

Functionalized nanoparticles may be administered to a patient as a composition or in combination with cells cultured in the presence of functionalized nanoparticles. An active agent or bolus of cells can be administered to an individual using any available method and route suitable for delivery, including in vivo and ex vivo methods, as well as systemic and localized routes of administration.

Conventional and pharmaceutically acceptable routes of administration include intranasal, intracranial, intracerebral, intracerebroventricular, intrathecal, epidural, subcutaneous, intradermal, intravenous, nasal, intraperitoneal, and other enteral and parenteral routes of administration. Routes of administration may be combined, if desired, or adjusted depending upon the niPSC product/or the desired effect. The composition can be administered in a single dose or in multiple doses. In some embodiments, the composition is administered intrathecally. In other embodiments, the composition is administered intravenously. In other embodiments, the composition is administered via an inhalational route. In other embodiments, the composition is administered intracranially.

The niPSC products can be administered to a host using any available conventional methods and routes suitable for delivery of conventional drugs, including systemic or localized routes. Administration may include, but are not limited to: ingestion, infusion, inhalation, irrigation, implantation or insufflation. In general, routes of administration contemplated by the disclosure include, but are not necessarily limited to, enteral, parenteral, or inhalational routes.

E3. Methods for Promoting Repair of a Genetic Mutation and Gene Editing

Within certain embodiments, the present disclosure provides methods for promoting the repair of genetic mutations within a target nucleic acid sequence or for use in gene editing applications. These methods include contacting the cell with a functionalized nanoparticle comprising a nanoparticle core to which is attached (a) a biologically active molecule that is selected from the group consisting of(i) a guide nucleic acid that is specific for said target nucleic acid, (ii) a nuclease that cleaves said target nucleic acid upon binding of said guide nucleic acid to said target nucleic acid, and (iii) a nucleic acid that encodes a nuclease that cleaves said target nucleic acid upon binding of said guide nucleic acid to said target nucleic acid and (b) a cell targeting molecule, including a cell membrane-penetrating molecule, such as an HIV Tat derived peptide or other peptide having, for example, from five to nine basic amino acids, including arginine and/or lysine. Within certain aspects, such functionalized nanoparticles may further include a donor nucleic acid molecule comprising a nucleotide sequence for insertion into the cleavage site of said target nucleic acid.

Within certain aspects of these methods, the functionalized nanoparticle may employ CRISPR technology and the nuclease may be a Cas9, nickase, or Ago nuclease, or a functional domain or a homolog thereof. Within these or related aspects of these methods, both the guide nucleic acid and the nuclease or nucleic acid encoding the nuclease are attached to the nanoparticle core. Within other aspects of these methods, only one of the guide nucleic acid and the nuclease or nucleic acid encoding the nuclease is attached to the nanoparticle core.

Functionalized nanoparticles using CRISPR or related gene editing technology for delivery to nanoparticle induced pluripotent stem cells (niPSCs). In certain aspects, the functionalized nanoparticles for editing and correcting genetic aberrations in niPSCs. For example, niPSCs derived from subjects having a genetic disease (exemplified herein by the genetic neurodegenerative disease ALSP) can be expanded in culture as described herein or as otherwise known in the art.

Such target genome modifying factors can be attached to the nanoparticles using various methods briefly described herein and as otherwise known in the art. See, e.g., U.S. Patent Publication No. 2014/0342004. The functionalized nanoparticles can be contacted to the niPSCs in the presence or absence of a magnetic field. The use of a magnet in case of contacting with superparamagnetic nanoparticles renders an important increase in the contact surface area between the cells and nanoparticles and thereby reinforces further improved penetration of functionalized nanoparticles through the cell membrane. Furthermore, applying a magnetic field after editing nucleotide sequence of the gene of interest in the cells aids in removal of functionalized nanoparticles from the treated cells which will further minimize the off-target effects of such gene editing thus preserving the genome integrity of the treated cells.

The cells are maintained attached or suspended in culture medium, and non-incorporated nanoparticles are removed by centrifugation or cell separation, leaving cells that are present as clusters. The cells are then resuspended and recultured in fresh medium for a suitable period. The cells can be taken through multiple cycles of separating, resuspending, and reculturing, until gene editing is confirmed prior to subsequent use of the cells in vitro or in vivo. The current method is applicable to introduce single or multiple nucleotide substitutions, deletions, insertions in the gene of interest or any gene-regulatory sequence, but also for introduction of premature truncation resulting in heterozygous or homozygous knock-out of the gene of interest. In one embodiment, the donor nucleotide sequence will comprise all or part of CSF-1R gene or the TREM2 gene.

This aspect of the disclosure overcomes the insertional mutagenesis and skewing genotype/phenotype problems of existing CRISPR/Cas9 and other genome editing approaches by using nanoparticle-based delivery of the requisite editing machinery. The nanoparticles can be metal-core (e.g., superparamagnetic iron-based—when rapid removal of nucleases using electromagnetic field is needed—or gold based nanoparticles) or non-cored (e.g., polymeric nanoparticles, such as liposomes or micelles) functionalized with any of the abovementioned or other bioactive molecules exposure to which may result in gene editing, i.e., targeted changes in the nucleotide sequence of genes of interest. The recited cell types, factors, and/or combinations of factors are not intended to be limiting and additional factors and/or combinations will be newly discovered and those combinations would work in the same way as described in the application.

The guide nucleic acid molecule, the modifying factor (e.g., nuclease such as Cas9, homologs, or functional derivatives thereof), and the donor nucleic acid molecule can all be conjugated to the same nanoparticle or alternatively, one or more of the aforementioned components can be conjugated to different nanoparticles in any combination. For example, the guide nucleic acid molecule and the modifying factor (e.g., nuclease) can be conjugated to the same nanoparticle whereas the donor nucleic acid molecule can be conjugated to a different nanoparticle. Alternatively, the guide nucleic acid molecule and the donor nucleic acid molecule can be conjugated to the same nanoparticle whereas the modifying factor (e.g., nuclease) can be conjugated to a different nanoparticle. Alternatively, the modifying factor (e.g., nuclease) and the donor nucleic acid molecule can be conjugated to the same nanoparticle whereas the guide nucleic acid molecule can be conjugated to a different nanoparticle. As yet another alternative, each of the three components can be conjugated to separate, individual nanoparticles. In any of the foregoing embodiments, the multiple nanoparticles can all be the same or different nanoparticle types, as described in more detail herein.

The present disclosure further provides methods for promoting the repair of a genetic mutation in a target nucleic acid, such as (1) a CSF-1R gene, including a CSF-1R gene having a genetic mutation in a region encoding a tyrosine kinase domain, (2) a TREM2 gene, and (3) an Arylsulfatase A (ARSA) gene.

Exemplified herein are functionalized nanoparticles for correcting CSF-1R mutations in cells, including induced pluripotent stem cells, which are derived from ALSP patients. In a further aspect of this method, one can use ALSP-specific niPSCs generated as described above and CRISPR/Cas9 technology well-known by those skilled in the art that permits knock-in of target sequences and single base pair substitution of interest (Jinek, Science 337:816-821 (2012); Sander, Nat. Biotechnol. 32:347-355 (2014); Chiba, Methods Mol. Biol. 1239:267-280 (2014); and Grobarczyk, Stem Cell Rev. 11:774-787 (2015).

One skilled in the art can use commercially available and validated in HEK293 cell plasmids containing CSF-1R-specific gRNA and Cas9 in one vector and a donor template with normal CSF-1R sequence in a separate plasmid. Such kits are available from various resources, such as from Applied Stem Cell (ASC), which has developed kits that successfully work in fibroblasts and in human pluripotent stem cells. One can select specific gRNAs for correction of CSF-1R mutations.

One skilled in the art can first transfect patient-specific niPSCs developed as described herein with corresponding custom-made plasmids. One can use a Neon electroporation system that has been optimized for ˜50-90% transfection efficiency and 90% viability of hiPSC or primary human fibroblasts using EGFP or RFP plasmids. Because of the presence of the puromycin-resistance gene in the gRNA/Cas9 plasmid, the transfected cells can be cultured in the presence of puromycin for 48 h for enrichment of transfected cells followed by single cell plating on matrigel or laminin coated plates for further expansion. The expanded clones can be evaluated by one skilled in the art for successful gene editing based on direct sequencing of PCR-amplified fragments. The successfully corrected clones can be further evaluated for potential off-target sites by similar DNA sequencing of hot-spots identified in the bioinformatic analysis. To date, the efficiency of CRISPR/Cas9 system in zebrafish and plants is up to 70%, whereas in human induced pluripotent stem cells it ranges from 2-5% (36-38). Therefore, one skilled in the art could examine up to 200 clones to identify 5-10 patient-specific niPSCs with a corrected CSF-1R mutation.

In order to deliver biologically active molecules intracellularly, the present disclosure provides a universal platform based on a composition including a cell membrane-penetrating nanoparticle with covalently linked biologically active molecules. To this end, presented herein is a functionalization method that ensures a covalent linkage of proteins, peptides, DNA and/or RNA molecules to nanoparticles. The modified cell-permeable nanoparticles of the present disclosure provide a universal mechanism for intracellular delivery of biologically active molecules for regulation and/or normalization of cellular function in general, and editing nucleotide sequences to correct or improve gene expression and function, which can be subsequently used in research and development, drug screening and therapeutic applications to improve cellular function in humans.

The methods disclosed herein utilize biocompatible nanoparticles, including for example, superparamagnetic iron oxide or gold nanoparticles, or polymeric nanoparticles similar to those previously described in scientific literature. See, e.g., Lewin et al., Nat. Biotech. 18:410-414 (2000); Shen et al., Magn. Reson. Med. 29:599-604 (1993); and Weissleder et al., Am. J. Roentgeneol. 152:167-173 (1989); each reference incorporated herein by reference in its entirety). Such nanoparticles can be used, for example, in clinical settings for magnetic resonance imaging of bone marrow cells, lymph nodes, spleen and liver. See, e.g., Shen et al., Magn. Reson. Med. 29:599 (1993) and Harisinghani et al., Am. J. Roentgenol. 172:1347 (1999).

For example, magnetic iron oxide nanoparticles sized less than 50 nm and containing cross-linked cell membrane-permeable TAT-derived peptide efficiently internalize into hematopoietic and neural progenitor cells in quantities of up to 30 pg of superparamagnetic iron nanoparticles per cell. See, Lewin et al., Nat. Biotech. 18:410-414 (2000).

Furthermore, the nanoparticle incorporation does not affect proliferative and differentiation characteristics of bone marrow-derived CD34+ primitive progenitor cells or the cell viability. Lewin et al., Nat. Biotech. 18:410-414 (2000). Accordingly, the disclosed nanoparticles can be used for in vivo tracking of the labeled cells, which can be useful when in vivo gene editing is used. The labeled cells retain their differentiation capabilities and can also be detected in tissue samples using magnetic resonance imaging. Disclosed herein are novel nanoparticle-based compositions, which are functionalized to carry various sets of RNA and/or DNA, proteins, peptides and other small molecules that can serve as excellent vehicles for intracellular delivery of biologically active molecules to target a specific nucleotide sequence of interest, introduce nucleotide sequence alterations of interest and thereby modulate cellular function and properties.

In another aspect, the present disclosure provides functionalized nanoparticles that may be used in methods for the modulation of intracellular activity via targeted editing of a nucleotide sequence to normalize/modify a gene sequence, control expression of a gene of interest, and/or introduce a new, heterologous gene for expression in the cell. For example, animal or human stem or other cell types, commercially available or obtained using standard or modified experimental procedures, are first plated under sterile conditions on a solid surface with or without a substrate to which the cells may adhere if needed (feeder cells, gelatin, martigel, fibronectin, and the like). The plated cells are cultured for a time with a specific factor combination that allows cell division/proliferation or maintenance of acceptable cell viability and concentration. Examples are serum and/or various growth factors as appropriate for the cell-type, which can later be withdrawn or refreshed and the cultures continued.

The plated cells are cultured in the presence of functionalized biocompatible cell-permeable nanoparticles with covalently linked target nucleotide sequence binding and modifying factors (that include but are not limited to peptide, DNA or RNA-based guiding molecules, a bi-functional or multifunctional enzyme with binding affinity to the guiding molecules and its nuclease activity, and, optionally, a donor nucleotide sequence necessary for gene correction) attached using various methods briefly described herein and elsewhere (see, e.g., U.S. Patent Publication No. 2014/0342004) in the presence or absence of a magnetic field. The use of a magnet in case of superparamagnetic nanoparticles renders an important increase in the contact surface area between the cells and nanoparticles and thereby reinforces further improved penetration of functionalized nanoparticles through the cell membrane. Furthermore, applying a magnetic field after editing a nucleotide sequence encoding the gene of interest in the cells aids in removal of functionalized nanoparticles from the treated cells which will further minimize the off-target effects of such gene editing, thus preserving the genome integrity of the treated cells.

The cells are maintained attached or suspended in culture medium, and non-incorporated nanoparticles are removed by centrifugation or cell separation, leaving cells that are present as clusters. The cells are then resuspended and recultured in fresh medium for a suitable period. The cells can be taken through multiple cycles of separating, resuspending, and reculturing, until gene editing is confirmed prior to subsequent use of the cells in vitro or in vivo. The current disclosure is applicable to introduce single or multiple nucleotide substitutions, deletions, insertions in the gene of interest or any gene-regulatory sequence, but also for introduction of premature truncation resulting in heterozygous or homozygous knock-out of the gene of interest. A broad range of cell types can be used such as human fibroblasts, blood cells, epithelial cells, mesenchymal cells, and the like.

Gene editing is based on the treatment of various cell types or tissues with bioactive molecules that can include various polypeptides, RNA and DNA molecules. Such bioactive molecules do not penetrate through a cell membrane efficiently, and may not reach the cell nuclei without a special delivery vehicle. Furthermore, these bioactive molecules have a short half-life and can undergo degradation upon exposure to various proteases and nucleases on route to a cell nucleus, which, altogether will result in a low gene editing efficiency overall. These disadvantages result in reduced efficacy of the bioactive molecules and therefore require much higher doses of a treatment to achieve a noticeable gene editing effect.

Therefore, in the current disclosure, functionalized nanoparticles are used to overcome the above-mentioned disadvantages. More specifically, these bioactive molecules, when linked to the nanoparticles and compared with the original “naked” state, acquire new physical, chemical, biological functional properties, that confer cell-penetrating and cell nucleus-targeting ability, larger size and altered overall three-dimensional conformation and the acquired capability to edit nucleotide sequence and/or expression of target gene(s) of interest. Since the first reports in 2013 demonstrating the suitability of the CRISPR/Cas9 nuclease system for gene editing in mammalian cells, many studies have been performed characterizing the mechanics and applicability of such editing systems. See, e.g., Cong et al. Science 339:819 (2013); Mali el al., Nat. Methods 10:957 (2013).

A number of guiding molecules and gene products with nuclease activity have subsequently been reported to exhibit gene editing effects, and the list continues to grow (Hsu et al., Cell 157:1262 (2014); Jiang et al., Appl. Fnviron. 81:2506 (2015); Doench et al., Nat Biolechnol. 32:1262 (2014); Tsai et al., Nat Biotechnol. 33:187 (2015); Fu el al., Methods Enzymol. 546:21 (2014); Wyvekens et al., Hum Gene Ther. 26:425 (2015); and Kim et al., Genome Res. 24:1012 (2014)).

As an example, RNA-based guiding molecules with affinity to the Cas9 nuclease and different moiety homologous to the targeted nucleotide sequence of interest and cDNA encoding the Cas9 nuclease with nuclear localization domain were introduced into the cells using electroporation or lipofection along with a template donor sequence. The guiding molecules bind the target sequence of cellular DNA and Cas9 nuclease creates a double stand break (“DSB”) (Choulika et al., Mol. Cell. Biol. 15:1968 (1995)) in the DNA at the specific position determined by the sequence of guiding RNA. Two such DSBs generate deletion of the region of interest that can be joined together by an internal mechanism of non-homologous end joining (“NHEJ”), thereby removing the nucleotide sequence of interest (Bibikova et al., Genetics 161:1169 (2002)).

Alternatively, in the presence of exogenous donor DNA template containing the correct nucleotide sequence with flanking nucleotide sequences homologous to the gene of interest region, a homologous recombination takes place resulting in insertion of the correct nucleotide sequence in the place of newly created deletion. This is referred to as “homology-derived recombination” (“HDR”) (Chu el al., Nat. Biotechnol. 33:543 (2015)).

Further variations of this gene editing approach include use of a nickase that is either an inactive nuclease (alone or fused or in combination with other bioactive molecules) that can alter target gene expression by virtue of binding to the target regulatory region of the gene and either activate or block its expression, or an active nuclease that creates single strand breaks (“SSB”), which is contrasted with the creation of DSB by Cas9. When used as a pair to target two nearby nucleotide sequences and in the presence of a donor sequence, the SSB can be repaired via HDR and exhibit lower (if any) non-specific off-target activity. The nickase can be represented by any enzyme like modified Cas9 or any fusion nickase enzyme generated by fusion of guiding molecule-binding domain of one gene (e.g., Cas9) with a nuclease domain of nickase (e.g., Fokl nuclease) described previously in Wyvekens et al., Hum Gene Ther. 26:425-431 (2015).

Because the off-target site binding of the nuclease (e.g., Cas9) is concentration dependent, a ribonucleoprotein particle (“RNP”) complex of the recombinant enzyme with guide-RNA has been generated for gene editing and can be introduced into the cells via electroporation or lipofection. As a result, the RNP can cleave the DNA and be rapidly degraded intracellularly, potentially resulting in lower off-target activity. See, Kim et al., Genome Res. 24:1012-1019 (2014). However, the off-target sites are still an issue with this approach due to the continuous presence of the RNP in the cells. The use of a magnetic field in the present disclosure for effective removal of non-integrating functionalized nanoparticles with active enzyme presents a unique way to rapidly withdraw the enzyme from the cells.

Alternative variations of this gene editing approach include the use of bioactive molecules with gene modifying activity. For example, acetylation of the lysine residues at the N-terminus of histone proteins removes positive charges, thereby reducing the affinity between histones and DNA. This makes RNA polymerase and transcription factors easier to access the promoter region. Therefore, in most cases, histone acetylation enhances transcription while histone deacetylation represses transcription. Such histone acetylation is catalyzed by histone acetyltransferases (HATs) and histone deacetylation is catalyzed by histone deacetylases (HDACs). DNA methylation is the addition of a methyl group (CH₃) to the DNA's cytosine base by methyltransferases that affect gene transcription. The methylation pattern is heritable after cell division, hence DNA methylation plays an important role in cell differentiation during development.

Potential problems with current gene editing approaches include premature degradation of the RNP, which may bind the target site but not cleave DNA due to intracellular proteolysis of the enzyme and lost nuclease activity. Such problems are addressed by the present disclosure, which, among other advantages, provides for the use of additional degradation-protecting compounds, such as a nanoparticle or a PEG or other compound or molecule functionalized with non-integrating peptides, proteins and RNA molecules and preserving the cell genome intact.

Furthermore, as indicated above, the established use of lentiviral vectors for delivery of guiding molecules and nucleases inside the cells is known to result in random integration of viral DNA into the human cell genome and may lead to detrimental consequences such as cancer. The present disclosure overcomes this problem upon generation and use of the nanoparticles functionalized using above-mentioned and/or other gene editing molecules as non-integrating complexes that preserve the cell genome intact.

In alternative strategies, current gene editing tools can also be based on the expression of gene products delivered to the cells using non-viral plasmid DNA. Again, the use of DNA is prone to trigger unpredictable random insertion of nucleotides into the genomic DNA of the host cell thereby potentially leading to detrimental consequences or skewing the phenotype. The present disclosure addresses this issue by presenting an innovative approach that is based on non-integrating multi-functional nanoparticles with cell-penetrating capacity with highly efficient delivery of components necessary for gene editing.

The present disclosure overcomes the insertional mutagenesis and skewing genotype/phenotype problems by using nanoparticles. The nanoparticles can be metal-core (e.g., superparamagnetic iron-based (when rapid removal of nucleases using electromagnetic field is needed) or gold based nanoparticles) or non-cored (e.g., polymeric nanoparticles, such as liposomes or micelles) functionalized with any of the above-mentioned or other bioactive molecules exposure to which may result in gene editing, i.e., targeted changes in the nucleotide sequence of genes of interest. The recited cell types, factors, and/or combinations of factors are not intended to be limiting, and additional factors and/or combinations will be newly discovered and those combinations would work in the same way as described in the application.

The guide nucleic acid molecule, the modifying factor (e.g., nuclease such as cas9, homologs, or functional derivatives thereof or other proteins with various activities), and/or the donor nucleic acid molecule can all be conjugated to the same nanoparticle or alternatively, one or more of the aforementioned components can be conjugated to different nanoparticles in any combination. For example, the guide nucleic acid molecule and the modifying factor (e.g., nuclease) can be conjugated to the same nanoparticle whereas the donor nucleic acid molecule can be conjugated to a different nanoparticle. Alternatively, the guide nucleic acid molecule and the donor nucleic acid molecule can be conjugated to the same nanoparticle whereas the modifying factor (e.g., nuclease) can be conjugated to a different nanoparticle. Alternatively, the modifying factor (e.g., nuclease) and the donor nucleic acid molecule can be conjugated to the same nanoparticle whereas the guide nucleic acid molecule can be conjugated to a different nanoparticle. As yet another alternative, each of the three components can be conjugated to separate, individual nanoparticles. In any of the foregoing embodiments, the multiple nanoparticles can all be the same or different nanoparticle types, as described in more detail herein.

The donor nucleotide sequence can be a DNA or RNA sequence that is intended to be inserted into (or have a portion thereof be inserted into) the target DNA or RNA molecule. This is useful for various applications, as described above, such as correcting a deleterious sequence in the cell genome. Such deleterious sequence can be, for example, a mutation resulting in a negative phenotype or an exogenous sequence from a pathogen. Alternatively, the donor nucleotide sequence can include a modified sequence to affect the expression levels ofa gene within the target genome. This can be, for example, providing a different or modified promoter sequence that enhances or reduces expression of the gene, but which does not otherwise modify the actual encoding sequence of the gene itself. As yet another example, the donor nucleotide sequence can introduce a heterologous encoding sequence (with or without a promoter sequence) to provide the cell the ability to express the heterologous gene and ultimately produce a new protein.

Another use of the disclosure is the screening/testing of a bioactive molecule (compound or compounds) for regulated gene editing and its expression. This involves combining the compound attached to the nanoparticle using methods disclosed herein with a cell population of interest (whether fibroblasts, blood cells, mesenchymal cells, and the like), culturing for suitable period and then determining any modulatory effect resulting from the compound(s). This includes knock-out of any gene product of interest, changes in nucleotide sequences of genes with one or more mutations whether those are single or multi-nucleotide substitutions, insertions, truncations or deletions to be further used for direct cell reprogramming and/or generation of specialized cell types of interest, such as cardiac cells, hepatocytes (liver cells), or neural cells, examination of the cells for toxicity, metabolic change, or an effect on contractile activity and/or otherfunction.

Another use of the described compositions is the formulation of specialized cells as a medicament or in a delivery device intended for treatment of a human or animal body. This enables the clinician to administer the non-integrating nanoparticles functionalized with gene editing molecules described above or other protein or RNA-based molecules in or around a tissue of interest (e.g., heart, bone marrow, brain or liver, etc.) either from the vasculature or directly into the muscle or organ wall, thereby allowing the specialized cells to engraft, limit the damage, and/or participate in regeneration/regrowth of the tissue's infrastructure and restoration of specialized function. Alternatively, the cells with an edited genome can be produced in vitro with the described functionalized nanoparticles, modified by targeted reprogramming into a special cell type of interest if needed, and administered thereafter into the area around diseased or damaged tissue of a subject.

Within further embodiments, the present disclosure provides methods for genome correction and modulation of cellular functions, which methods comprise contacting a cell or administering to a patient one or more functionalized nanoparticles as disclosed herein.

### EXAMPLES

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

### Example 1

### Linking of Green Fluorescent Protein (GFP) to Superparamagnetic Nanoparticles with an LC-SMCC Crosslinker

This Example demonstrates the linking of green fluorescent protein (GFP) to superparamagnetic nanoparticles via a long-chain variant of the crosslinker succinimidyl trans-4-(maleimidylmethyl)cyclohexane-1-carboxylate (SMCC).

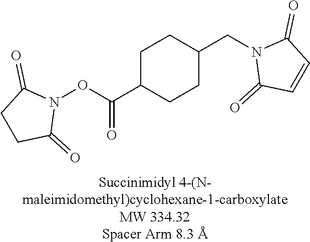 [SMILES: O=C(ON1C(=O)CCC1=O)C1CCC(CN2C(=O)C=CC2=O)CC1]

LC-SMCC (Succinimidyl-4-[N-maleimidomethyl]cyclohexane-1-carboxy-[6-amidocaproate]) is a long-chain variant of SMCC having a spacer length of 16.2 Å.

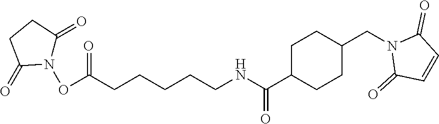 [SMILES: O=C(CCCCCNC(=O)C1CCC(CN2C(=O)C=CC2=O)CC1)ON1C(=O)CCC1=O]

SMCC is an amine-to-sulfhydryl crosslinker that contains NHS-ester and maleimide reactive groups at opposite ends of a medium-length cyclohexane-stabilized spacer arm (8.3 angstroms). Succinimidyl-4-(N-maleimidomethyl)cyclohexane-1-carboxylate (SMCC) is a non-cleavable and membrane permeable crosslinker. It contains an amine-reactive N-hydroxysuccinimide (NHS ester) and a sulfhydryl-reactive maleimide group. NHS esters react with primary amines at pH 7-9 to form stable amide bonds. Maleimides react with sulfhydryl groups at pH 6.5-7.5 to form stable thioether bonds. The maleimide groups of SMCC and Sulfo-SMCC and are unusually stable up to pH 7.5 because of the cyclohexane bridge in the spacer arm.

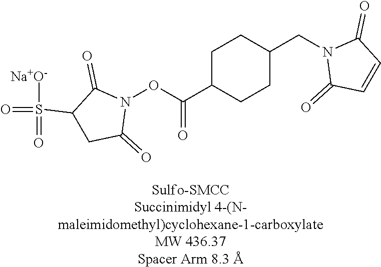

Two-step reaction sequence for crosslinking biomolecules using the heterobifunctional crosslinker SMCC:

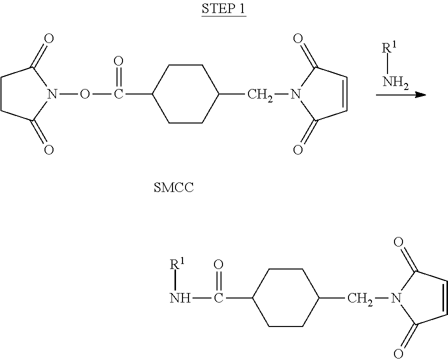

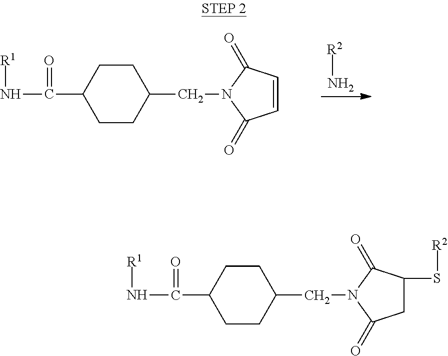

GFP (Clontech Laboraties, Inc., Mountain View, Calif.) was linked to the superparamagnetic nanoparticles using the amine-to-sulfhydryl crosslinker, LC-SMCC (from Fisher Scientific, Pittsburgh, Pa.). LC-SMCC was attached to amino groups on the superparamagnetic nanoparticles, and (?) was then coupled directly to the sulfhydryl groups on GFP.

LC-SMCC was dissolved in dimethylformamide (DMF; ACROS Organics, Fisher Scientific) in a sealed and anhydrous container at a concentration of 1 mg/ml. Ten microliters (10 μl) of the LC-SMCC/DMF solution was added immediately to the superparamagnetic nanoparticles in a final volume of 200 μl, thereby providing a large molar excess of LC-SMCC over the amine groups present on the superparamagnetic nanoparticle surface. This reaction was allowed to proceed for one hour, after which time excess LC-SMCC and DMF was removed using an Amicon spin filter with a cutoff of 3,000 Da (EMD Millipore Corporation, Billerica, Mass.). To ensure that excess LC-SMCC was removed, five exchanges of volume were performed to achieve proper buffer exchange.

Peptide-based molecules (including commercially available Aequorea Victoria green fluorescent protein (GFP), purified recombinant GFP, or other proteins) were added to a solution containing ethylene glycol for freezing at ˜30° C. To 3 μg of protein in 14 μl, 10 μl of a freshly prepared dithiothreitol (DTT, Cleland's reagent) solution in phosphate-buffered saline (PBS) was added with vigorous vortexing. Because the proteins usually contain more than one cysteine, there was a tendency to crosslink different GFP molecules. Excess DTT reduced the dithiol linkage thereby preventing GFP crosslinking. Reactions were allowed to proceed for two hours at 40° C.; excess reagent was then removed by an Amicon centrifugal filter unit with a 3,000 MW cutoff.

The activated nanoparticles and protein solutions were combined and allowed to react for two hours, after which the unreacted protein was removed with an Amicon centrifugal filter unit having an appropriate MW cutoff (e.g., for GFP a 50,000 Da cutoff was employed). Sample was stored at −80° C. A sulfo-derivative of SMCC (Sulfo-SMCC), which exhibits greater water solubility than LC-SMCC, can be used. Anhydrous DMSO may also be substituted for anhydrous DMF as the solvent carrier for the labeling reagent.

### Example 2

### Linking of Green Fluorescent Protein (GFP) to Superparamagnetic Nanoparticles with an LC-SPDP Crosslinker

This Example demonstrates the linking of green fluorescent protein (GFP) to superparamagnetic nanoparticles via a LC-SPDP (Succinimidyl 6-(3-[2-pyridyldithio]-propionamido)hexanoate), which is a heterobifunctional, thiol-cleavable, and membrane permeable crosslinker. SPDP contains an amine-reactive N-hydroxysuccinimide (NHS) ester that reacts with lysine residues to form a stable amide bond. The other end of the spacer arm is terminated in the pyridyl disulfide group that will react with sulfhydryls to form a reversible disulfide bond. crosslinker.

SPDP is a short-chain crosslinker for amine-to-sulfhydryl conjugation via NHS-ester and pyridyldithiol reactive groups that form cleavable (reducible) disulfide bonds with cysteine sulfhydryls. LC-SPDP is a long-chain crosslinker for amine-to-sulfhydryl conjugation via NHS-ester and pyridyldithiol reactive groups that form cleavable (reducible) disulfide bonds with cysteine sulfhydryls.

Three analogs of SPDP can be employed according to the methodology described in this Example: the standard version (SPDP), a derivative with a longer spacer arm (LC-SPDP), and a sulfonated water-soluble variety (Sulfo-LC-SPDP).

SPDP reacts with an amine-containing biomolecule at pH 7 to 9, yielding a pyridyldithiopropionyl mixed disulfide. The mixed disulfide can then be reacted with a reducing agent such as DTT or TCEP to yield a 3-mercaptopropionyl conjugate or with a thiol-containing biomolecule to form a disulfide-linked tandem conjugate. Either reaction can be quantified by measuring the amount of 2-pyridinethione chromophore released during the reaction.

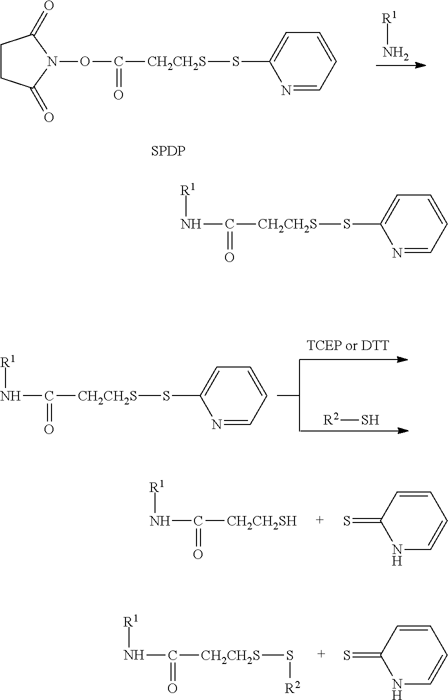

In this method, the amino groups of lysine were used for the coupling reaction to sulhydryl groups on the bead. Beads freshly equilibrated with 0.1 M phosphate buffer at pH 7.2 were used in these studies. LC-SPDP at 1 mg/ml (in DMF) was freshly prepared. Ten (10) μl of SPDP solution was added to the bead suspension under vigorous vortexing and allowed to react for one hour. Subsequently, the unreacted material was removed by centrifugation and the nanoparticles washed with phosphate buffer using an Amicon Spin filter with a 10K cutoff. The disulfide bond of SPDP was broken using Clelands reagent; 1 mg was added to the solution and the reaction allowed to proceed for one hour. By-products and unreacted Clelands reagent were removed via an Amicon spin filter with a 10K cutoff.

While the above reaction proceeded, GFP was blocked using N-ethylmaleimide. Excess N-ethylmaleimide was added to the GFP solution. Reaction proceeded for one hour at room temperature and unwanted materials removed using an Amicon Spin filter with a 3K cutoff. The GFP was then allowed to react with excess SMCC for one hour.

Subsequently, GFP was purified on a spin column and then reacted with PDP-nanoparticles. The reaction proceeded for one hour and the final product purified using an Amicon spin filter with a cutoff of 50K.

### Example 3

### Intracellular Delivery of a Biologically Active Protein Using Functionalized Nanoparticles

This Example demonstrates that functionalized nanoparticles of the present disclosure can successfully deliver a protein intracellularly, in this case GFP for sorting purposes, and confer upon said cell a novel property, in this case green fluorescence, while maintaining an intact cellular genome and the integrity of cellular DNA.

Human fibroblasts, which were either obtained commercially or using standard experimental procedures as described in Moretti et al., FASEB J. 24:700 (2010), were plated under sterile conditions at 150,000 cells per well in six-well plates with or without 150,000-200,000 preplated feeder cells, which feeder cells were either obtained commercially or using standard experimental procedures. The plated cells were cultured with a specific factor combination that allows cell division/proliferation or maintenance of acceptable cell viability in serum-containing culture medium, which can later be withdrawn or refreshed and the cultures continued under sterile conditions in a humidified incubator with 5% CO₂ and ambient O₂.

The cells collected at the bottom of a conical tube or the plated cells were treated with 50 μl of a suspension containing superparamagnetic functionalized nanoparticles (SPBN) with bioactive molecules attached using various methods disclosed herein in the presence or absence of magnetic field.

The use of magnetic field in case of superparamagnetic nanoparticles renders an important increase in the contact surface area between the cells and nanoparticles and thereby ensuring improved penetration of functionalized nanoparticles through the cell membrane. Importantly, similar to polyethylene glycol (PEG)-mediated protection of several protein-based drugs (PEG-GCSF, Amgen, Calif.; PEG-Interferon, Schering-Plough-Merck, N.J.) to which PEG is attached, the nanoparticles used in conjunction with coupled peptides increase the size of the polypeptide and masks the protein's surface, thereby reducing protein degradation by proteolytic enzymes and resulting in a longer stability of the protein molecules used. If necessary, the cell population is treated repeatedly with the functionalized nanoparticles to deliver the bioactive molecules intracellularly.

The cells are suspended in culture medium, and non-incorporated nanoparticles are removed by centrifugation for 10 minutes at approximately 1200×g, leaving cells that are present as clusters in the pellet. The clustered cells are then resuspended, washed again using similar procedure and recultured in fresh medium for a suitable period. The cells can be taken through multiple cycles of separating, resuspending, and reculturing in a culture media until a consequent biological effect triggered by the specific bioactive molecules delivered intracellularly is observed.

In this specific example with green fluorescent protein, the cell-penetrant nanoparticles deliver the protein inside the cells, which confers acquisition of novel green fluorescence by the target cells. This newly acquired property allows subsequent sorting and separation of the cells with intracellularly delivered protein to high degree of homogeneity that can be further used for various applications. Importantly, the use of cell-permeable functionalized nanoparticles with attached protein devoid any integration into the cell genome, thereby ensuring that every cell with novel (in this case fluorescent) property maintains intact genome and preserves the integrity of cellular DNA.

### Example 4

### Functionalized Gold Nanoparticles and Gold Binding Peptides for Use in Cellular Reprogramming

This Example demonstrates that gold nanoparticles in combination with gold binding peptides may be employed to generate functionalized nanoparticles that are suitable for use in reprogramming a cell.

The present disclosure provides membrane-transducing peptides and transcription factors linked to gold nanoparticles via gold-binding peptide (GBP) for the reprogramming of cells. The disclosure describes the production of recombinant fusion proteins consisting of a His tag for purification, a transducing peptide to cross the cell membrane, transcription factors to facilitate entry into the nucleus and initiation of transcription to reprogram cells, and a high affinity gold binding peptide due to the presence of one to several repeats to bind the fusion protein to gold nanoparticles. The disclosure further describes the use of the functionalized gold nanoparticles for the reprogramming of cells into other lineages including pluripotent stem cells.

A seven peptide repeat gold binding peptide (GBP) was used as described by Brown. Nature Biotechnology 15:269-272 (1997). Adaptations of GBP are disclosed by Furlong and Woodbury in U.S. Pat. No. 6,239,255 that may be used in combination with one or more sensors by excising the nucleotide sequence encoding GBP from the plasmid pSB3053 from Brown, Nature Biotech 15:269-272 (1997) and ligating it into suitable vectors.

The nucleotide and amino acid sequences of a 7-repeat GBP are presented in Woodbury, U.S. Patent Publication No. 2005/0106625 and are presented herein as SEQ ID NOs: 1 and 2, respectively. See, Table 2. The GBP is expressed with fusion proteins and His tag at either the N or C-terminus.

TABLE 2
Nucleotide and Amino Acid Sequences of a 7-repeat GBP

| Sequence | | |
|---|---|---|
| Identifier | Description | Sequence |
| SEQ ID NO. 1 | Nucleotide | atg cat gga aaa act cag gca ace age ggg act atc cag agc |
| | Sequence of a | atg cat gga aaa act cag gca acc agc ggg act atc cag agc |
| | 7-repeat GBP | atg cat gga aaa act cag gca acc agc ggg act atc cag agc |
| | | atg cat gga aaa act cag gca acc agc ggg act atc cag agc |
| | | atg cat gga aaa act cag gca acc agc ggg act atc cag agc |
| | | atg cat gga aaa act cag gca acc agc ggg act atc cag agc |
| | | atg cat gga aaa att cag gca acc agc ggg act atc cag agc |
| SEQ ID NO. 2 | Amino Acid | Met His Gly Lys Thr Gln Ala Thr Ser Gly Thr Ile Gln Ser |
| | Sequence of a | Met His Gly Lys Thr Gln Ala The Ser Gly The Ile Gln Ser |
| | 7-repeat GBP | Met His Gly Lys Thr Gln Ala Thr Ser Gly Thr Ile Gln Ser |
| | | Met His Gly Lys Thr Gln Ala Thr Ser Gly Thr Ile Gln Ser |
| | | Met His Gly Lys Thr Gln Ala Thr Ser Gly Thr Ile Gln Ser |
| | | Met His Gly Lys Thr Gln Ala Thr Ser Gly Thr Ile Gln Ser |
| | | Met His Gly Lys Ile Gln Ala Thr Ser Gly Thr Ile Gln Ser |

For expression of the GBP fusion protein, there are a number of possible plasmid configuration combinations which vary the positions of GBP, transcription factor, poly His tag, and cell penetrating peptide.

### Example 4

### Design of GBP Fusion Protein Expression Vectors

A vector for protein fusion expression is chosen for expression in the system of choice, which may include bacteria, yeast, fungi, baculovirus, plant, or mammalian cells. The vector encodes a selectable marker and has the capacity to replicate in bacteria as well as in the system of choice. The vector will promote transcription initiation and termination of the fusion expression unit and this transcription is preferably regulated. The construct may include a leader sequence for secretion or may not do so for intracellular expression.

Herein described is the design of vectors for the expression of GBP fusion proteins in bacteria (E. coli). A variety of bacterial expression vectors are known in the art but pET16b was chosen for thise exampl. The elements of the fusion protein include a poly histidine tag, 7 repeats of GBP, a transcription factor that include Oct 4, Sox 2, Nanog, or Lin 28, and a transducing peptide. The transducing peptide may be a number of different peptides known in the art, but a 12 amino acid arginine (R12) is being used for this example. The elements of the fusion protein may be in different order, but those used were: (1) Poly His tag, GBP, transcription factor, R12 and (2) R12, transcription factor, GBP, poly His tag

Each of the transcription factors; Oct 4, Sox 2, Nanog, or Lin 28 are configured in these two expression units for a total of 8 constructions. These expression units are synthesized by GenScript and inserted into the pET16B vector. These constructs are transfected into bacteria under antibiotic selection, single colonies selected, grown in liquid culture, and frozen at −80° C. as glycerol stocks.

### Example 5

### Expression of GBP Fusion Proteins

To assess the utility of each of the GBP fusion protein constructions, an overnight culture was started from frozen glycerol stocks. A 1 ml aliquot of overnight was added to a 25 ml culture of LB with ampicillin and incubated at 37° C. until the O.D. 600 was 0.6. Aliquots of these un-induced cells were taken, pelleted, frozen, and saved to be processed along with induced samples. For the remainder of the cultures, incubation temperature was reduced to 30° C., IPTG was added to 50 uM and incubation continued for 20 hours. The cells were collected by centrifugation, washed once with 150 mM KCl, and frozen. The frozen cells of un-induced and induced samples were lysed with 1 ml of B-PER (a gentle lysis buffer for release of soluble proteins (Pierce Thermo Scientific)) and 10 minute incubation at room temperature. The solution was clarified with centrifugation at 16,000×G for 15 minutes. The pellet was extracted directly with SDS-PAGE sample buffer to recover insoluble proteins. Aliquots of the uninduced and induced samples were analyzed side-by-side by SDS-PAGE analysis and staining with colloidal form of coomasie blue (Invitrogen). Depending on the configuration of the fusion proteins being expressed, they were either soluble or insoluble.

### Example 6

### Purification of GBP Fusion Proteins

Larger cultures were grown to obtain larger amounts of fusion proteins. To release proteins that were all or substantially all produced in a soluble form as opposed to inclusion bodies, extraction from the cells was performed under “native” conditions for subsequent purification. The bacteria were resuspended to a final density approximately 20 times greater than that of the original cultures in ×50 mM sodium phosphate buffer, pH 8.0, containing 0.5M sodium chloride, 10 mM imidazole, protease inhibitor PMSF, and commercial cocktail of protease inhibitors. Cells on ice were lysed by sonication at medium power and interval setting of 50% to give an intermittent pulse of 30 seconds. This was repeated for 6 cycles with one-minute rest on ice between cycles. Following each cycle, the OD 600 nm was determined to assess cell lyses. The sonicated suspension was centrifuged 5,000×G for 10 min to remove cell debris and insoluble proteins. The resulting preparation was ready for His-tag affinity purification.

For those fusion proteins produced as inclusion bodies but with appropriate secondary structure of each fusion protein molecule within the inclusion body (modified from Singh and Panda, J. Biosci. and Bioeng. 99:303-310 (2005)). This procedure includes purifying and gently solubilizing the inclusion bodies to maintain secondary structure of the fusion proteins. Producing cells were centrifuged, washed with 50 mM Tris-HCl, pH 8.5, 5 mM EDTA (TE buffer), sonicated for 8 cycles as described in preceding paragraph, centrifuged 12,000 rpm, 20 min, at 4° C., and resuspended in TE buffer with 1% sodium deoycholate (TED buffer). Washing the inclusion bodies with TED buffer, sonication, and centrifugation was repeated three times, resulting in pure inclusion bodies in the final pellet.

The pure inclusion bodies were resuspended in 100 mM Tris-HCl, pH 12.5, 2 M urea, and incubated at 30° C. with stirring for two hours. The solubilized fusion proteins were incrementally diluted (1/10^{th} volume every 10 minutes) with refolding buffer 50 mM Tris-HCl, pH 8.0, 0.5 mM EDTA, 2 M urea, 10% glycerol, 5% sucrose, 1 mM PMSF, at 4 degrees C., with constant stirring, until the pH was 8.0 and then the solution was incubated for 1 additional hour. The solution was adjusted to 10 mM imidazole and was ready for poly histidine column purification.

For those fusion proteins that are not amendable to the first two methods of purification, a total denaturation and refolding method was used (modified from Tichy et al., Protein Expression and Purification 4:59-63 (1993)). Inclusion bodies purified as described above were solubilized in 8 M urea, 50 mM KH₂PO₄, pH 12.5, 1 mM EDTA, 50 mM NaC, 30 degrees C., for 2 hours with constant stirring. The solubilized fusion proteins were incrementally diluted (1/10^{th} volume every 10 minutes) with refolding buffer 50 mM Tris-HCl, pH 8.0, 0.5 mM EDTA, 2 M urea, 10% glycerol, 5% sucrose, 1 mM PMSF, at 4° C., with constant stirring, until the pH was 8.0 and then the solution was incubated for one additional hour. The solution was adjusted to 10 mM imidazole and was ready for poly histidine column purification.

The His-tag recombinant proteins were purified on ProBond nickel-resin (Invitrogen) as recommended by the manufacturer. Material in the two extracts, i.e., under native conditions for soluble proteins or denaturing conditions for insoluble proteins, was incubated with individual Probond Nickel resin columns, washed, and eluted as recommended by the manufacturer. The optical density at 280 nm of the eluate fractions was recorded and the peak fractions from each column were pooled, aliquoted and stored at −20° C. Purification usually produces fusion protein with 90 to 95% purity as assessed by SDS-PAGE.

### Example 7

### Addition of GBP Fusion Proteins to Gold Nanoparticles

Gold nanoparticles 5 nm to 25 nm were obtained from Nanopartz. To prepare gold nanoparticles for attachment of GBP fusion proteins, the gold nanoparticles were treated 3 times with 1.0 mL of 10 mM potassium phosphate pH 7.0, 10 mM KCl, and 1% Triton X-100 (PKT buffer) in Eppendorf centrifuge tubes at 100° C. for 20 min with frequent mixing to maintain gold suspension. The gold nanoparticles were then rinsed with PKT buffer at RT three times alternating resuspension and centrifugation.

To assess the attachment of GBP fusion proteins to the gold nanoparticles, the GBP fusion protein was labeled with FITC. A serial dilution of the FITC labeled fusion protein was added to a fixed amount of gold nanoparticles in PKT buffer and incubated for 15 min with frequent mixing. The gold nanoparticles were washed three times with PKT buffer by alternating resuspension and centrifugation and the fluorescence determined by a 96 well fluorometer. Thus, the optimal amount of GBP fusion protein per amount of gold nanoparticles was determined.

Somatic cell reprogramming into stem cells using these functionalized gold nanoparticles was confirmed by the methodology as described herein for functionalized paramagnetic nanoparticles.

### Example 8

### Functionalized Nanoparticles for and Reprogramming Methods for Producing Cardiomyocye-Like Cells (iCMs)

### Prophetic

This Example discloses the use of non-integrating nanoparticles are functionalized with cardiac-specific transcription factors including Gata4, MEF2C, TBX5, MESP1, and MYOCD as described in Nam et al., Proc. Natl. Acad. Sci. USA. 110:5588-5593 (2010).

Briefly, the human somatic cells are treated with functionalized nanoparticles once or repeatedly (2 or more times), which results in delivery of cardiac-specific factors to the cytoplasm and nucleus of the treated cells. The cells are maintained in appropriate culture medium for extended period of time and the outcome of such direct reprogramming of human somatic cells into functional cardiac cells is monitored using various molecular biology, biochemistry and cell biology techniques. Specifically, expression of cardiac specific Troponin T or tropomyosin can be determined by RNA isolation followed by real time or reverse transcribed PCR, immunostaining of the cells using appropriate antibodies, or by flow cytometry analyses of the cultured cells.

### Example 9

### Functionalized Nanoparticles for and Reprogramming Methods for Producing Cardiomyocye-Like Cells (iCMs)

### Prophetic

This Example discloses the use of non-integrating nanoparticles are functionalized with cardiac-specific transcription factors including Gata4, Hand2, TBX5, MYOCD and two microRNAs miR-1 and miR-133.

A different set of cardiac specific factors for direct reprogramming of human somatic cells can include nanoparticles functionalized with cardiac-specific transcription factors and RNAs (including mRNAs, microRNAs, shRNAs, and siRNAs), including, for example, the proteins Gata4, Hand2, TBX5, MYOCD and the RNAs (including mRNAs, microRNAs, shRNAs, and siRNAs) such as the microRNAs miR-1 and miR-133. This combination of bioactive molecules was introduced into the cells using viral vectors is efficient in direct reprogramming of human fibroblasts with generation of functionally active and contracting cardiomyocyte-like cells by Wada et al., Proc. Nal. Acad. Sci. USA. 110:12667-12672 (2013).

Here, the human fibroblasts are treated with nanoparticles functionalized with set 2 of recombinant proteins and RNAs (including mRNAs, microRNAs, shRNAs, and siRNAs) and cultured to induce generation of human iCMs. Alternative combination of these and/or other sets of cardiac-specific factors that together trigger reprogramming of human somatic cells into cardiac cells.

The preparation of these non-integrating functionalized nanoparticles does not involve any DNA molecules that could integrate into the cell genome and disrupt normal gene expression pattern. The present disclosure may be embodied in other specific forms without departing from the spirit or essential characteristics thereof.

The recited cell-types, factors, and/or combinations of factors are illustrative and are not intended to be limiting. Additional factors and/or combinations, including those that are newly discovered, are encompassed in this disclosure and will function the same way as described herein.

### Example 10

### Functionalized Nanoparticles and Reprogramming Methods for Producing Hepatocyte Cells (iHCs)

### Prophetic

This Example discloses the use of non-integrating nanoparticles are functionalized with hepatocyte-specific transcription factors including FOXA3, HNF1A, and HNF4A.

Non-integrating nanoparticles are functionalized with hepatocyte-reprogramming transcription factors as described in Huang et al., Cell Stem Cell 14:370-384, (2014). Briefly, the human somatic cells are treated with functionalized nanoparticles once or repeatedly (2 or more times), which results in delivery of liver-specific factors to the cytoplasm and nucleus of the treated cells.

The cells are maintained in appropriate culture medium for extended period of time and the outcome of such direct reprogramming of human somatic cells into functional liver cells is monitored using various molecular biology, biochemistry and cell biology techniques. Specifically, expression of albumin (ALB), a-1-antitrypsin (AAT) and cytochrome P450 (CYP) enzymes can be determined by RNA isolation followed by real time or reverse transcribed PCR, immunostaining of the cells using appropriate antibodies, or by flow cytometry analyses of the cultured cells. Furthermore, the functionality of the newly generated hepatocytes can also be confirmed by evaluating metabolic activity of induced CYP enzymes using liquid chromatography-tandem mass spectrometry.

These type of hepatic cells, albeit reprogrammed using lentiviral vectors, show restoration of liver function in an animal model of acute liver failure (Huang et al., Cell Stem Cell 14:370-384 (2014)). The recited cell-types, factors, and/or combinations of factors are illustrative and are not intended to be limiting. Additional factors and/or combinations, including those that are newly discovered, are encompassed in this disclosure and will function the same way as described herein.

### Example 11

### Functionalized Nanoparticles and Reprogramming Methods for Producing Neuronal Cells (iNCs)

### Prophetic

This Example discloses the use of non-integrating nanoparticles are functionalized with neuronal cell-specific transcription factors including SOXA2 and PAX6.

The non-integrating nanoparticles are functionalized with a set of neural-reprogramming transcription factors PAX6 and/or SOX2 as described in Connor, Protocol Exchange doi: 10.1038/protex.2015.034 (2015). Briefly, the human somatic cells are treated with functionalized nanoparticles once or repeatedly (2 or more times), which results in delivery of the reprogramming factors to the cytoplasm and nucleus of the treated cells.

The cells are maintained in appropriate culture medium for extended period of time and the outcome of such direct reprogramming of human somatic cells into neural progenitor cells is monitored using various molecular biology, biochemistry and cell biology techniques. Specifically, expression of neuron-specific TUJ1, MAP2, or NSE phenotypic markers together with tyrosine hydroxylase (TH), vGlut1, GAD65/67 and DARPP32 in the newly generated neural cells can be determined by RNA isolation followed by real time or reverse transcribed PCR and/or immunostaining of the cells using appropriate antibodies, or by flow cytometry analyses of the cultured neural cells reprogrammed directly from human fibroblasts.

The recited cell-types, factors, and/or combinations of factors are illustrative and are not intended to be limiting. Additional factors and/or combinations, including those that are newly discovered, are encompassed in this disclosure and will function the same way as described herein.

### Example 12

### A Pharmaceutical Screen Using Patient-Specific Cardiac Cells Generated Via Functionalized Nanoparticle Directed Reprogramming of Somatic Cells

### Prophetic

This Example discloses the use of functionalized nanoparticles in a pharmaceutical screen that utilizes patient-specific cardiac cells generated by the direct somatic cell reprogramming.

It is well-established that people react differently to pharmaceutical drugs. These differences can manifest at the cellular level because their cells react differently to pharmaceutical drugs based on genotype or variant developmental histories of cells among individuals. See, Turner et al., Rev Syst Biol Med. 7(4):221-41 (2015). Germline variants are inherited variations and are often associated with the pharmacokinetic behavior of a drug, including drug disposition and ultimately drug efficacy and/or toxicity, whereas somatic mutations are often useful in predicting the pharmacodynamic response to drugs.

Pharmacoethnicity, or ethnic diversity in drug response or toxicity, is an increasingly recognized factor accounting for interindividual variations of drug response. Pharmacoethnicity is often determined by germline pharmacogenomic factors and the distribution of single nucleotide polymorphisms across various populations. See, Patel, Pharmacogenet Genomics 25(5):223-30 (2015).

Thus, a pharmaceutical screen that utilizes patient-specific cardiac cells generated upon direct reprogramming of patients' somatic cells will reflect biases that are due to the individual's unique reaction to the pharmaceutical drugs. It may be that initial drug screens may be performed with cells from one source or individual but to broaden the applicability of a drug to the general population; a much wider selection of cells from different individuals is needed. The larger the number of source individuals the greater the probability the drug is going to have uniform response in the general population. Without this wider screening effort the drug may be effective for only a percentage of the population, for example 50, 40, or 20%, with this percentage reducing the profitability of a drug. The larger the number of source individuals for generation of cardiac cells used in drug screening, the greater the percentage of people being effectively treated with a given drug.

Similarly, participants in clinical trials may be pre-qualified for a clinical trial with a cellular assay with cardiac cells produced upon direct reprogramming of somatic cells of the candidate participant. If the cells respond well to the drug being assessed in the clinical trial the individual would be included in the clinical trial. If the cells did not respond well, the individual may be excluded from the trial. With pre-validation of the participants' better outcomes of the clinical trial may be assured.

### Example 13

### Knock-Out of a Programmed Cell Death Protein 1 (PD-1) Gene with Non-Integrating Functionalized Nanoparticles

### Prophetic

This Example discloses non-integrating functionalized nanoparticles for gene editing in an application comprising the knock-out of a programmed cell death protein 1 (PD-1) gene.

Programmed cell death protein 1 (PD-1; a/k/a cluster of differentiation protein 279 (CD279)) is a protein, which in humans is encoded by the PDCD1 gene. See, Shinohara et al., Genomics 23:704-6 (1994) and the NCBI full report on PDCD1 “Programmed cell death 1 [Homo sapiens (human)]; Gene ID: 5133, updated on 9 Oct. 2016.

PD-1 is a cell surface receptor that binds the two ligands PD-L1 and PD-L2 and functions as an immune checkpoint. PD-1 plays an important role in down regulating the immune system by preventing the activation of T-cells, which in turn reduces autoimmunity and promotes self-tolerance. The inhibitory effect of PD-1 is accomplished through a dual mechanism of promoting apoptosis (programmed cell death) in antigen specific T-cells in lymph nodes while simultaneously reducing apoptosis in regulatory T cells (suppressor T cells). See, Francisco et al., (July 2010). The PD-1 pathway and its role in tolerance and autoimmunity are reviewed in Immunological Reviews 236:219-42 (2010) and Fife and Pauken, Ann. N.Y. Acad. Sci 1217:45-59 (2011).

Compounds that block PD-1 functionality (i.e., PD-1 inhibitors) activate the immune system to attack tumors and are therefore used with varying success to treat some types of cancer. See, Schumann et al., Proc. Natl. Acad. Sci. U.S.A. 112:10437-42 (2015).

Non-integrating functionalized nanoparticles can be used to turn off the PD-1 gene expression in target cells as an attractive potent alternative to PD-1 inhibitors. For attachment of Cas9 nuclease and guiding nucleic acid molecules, various routes of functionalization can be used with one of such routes presented below. Nuclease Cas9 is linked to the nanoparticle (can be superparamagnetic, gold or polymeric nanoparticle) using LC-SMCC as the cross linker chain (LC1, attached to the amine groups of the nanoparticle), which is then coupled directly to the sulfhydryl group on Cas9. LC-SMCC (from Thermo Fisher) is dissolved in dimethylformamide (DMF) obtained from ACROS (sealed vial and anhydrous) at the 1 mg/ml concentration. Sample is sealed and used almost immediately.

One (1) to ten (10) microliters of the solution are added to nanoparticles in 200 microliter volume, which provide various excess ratio of SMCC to the available amine groups present, and the reaction is allowed to proceed for one hour. Excess SMCC and DMF can be removed using an Amicon spin filter with a cutoff of 3,000 daltons. At least five exchanges of volume are required to ensure proper buffer exchange. It is crucial that excess of LC1 (SMCC) be removed at this stage. Subsequently, a cell-penetrating peptide with terminal cysteine residue (described in WO/2013/059831, incorporated herein by reference in its entirety) is allowed to react with SMCC on nanoparticle and the non-bound peptide is removed by at least five washes using Amicon spin filters described above. At this stage, some amine groups on nanoparticles will remain intact, thereby providing docking sites for covalent attachment of second different length linker chain (LC2), which is attached using the same procedure described above for SMCC. Again, it is crucial that excess of LC2 be removed at this stage.

The Cas9 nuclease with a terminal cysteine is pre-incubated 10 min at 37° C. with PD-1 specific guiding RNA molecules (gRNAs) as described in Schumann et al., Proc. Nat'l. Acad. Sci. U.S.A. 112:10437-42 (2015) or added to a nanoparticle along with gRNAs with homology to a target sequence of PD-1 in a 1:1 ratio and the reaction is allowed to proceed for two hours at 4° C.

The excess reagent is removed by passing the functionalized superparamagnetic nanoparticles using available appropriate size columns and magnet from different vendors, such as Myltenyi Biotech, and the resultant product is used for treatment of target cells.

The human primary T cells isolated either from fresh whole blood or buffy coats as described (Schumann et al., Proc. Nat'l. Acad. Sci. U.S.A. 112:10437-42 (2015)) are treated with non-integrating cell-penetrant nanoparticle functionalized with Cas9 nuclease and target-specific gRNAs. Briefly, 100,000 cells cultured under sterile conditions on a solid surface in a humidified incubator with 5% CO₂ and ambient O₂ are treated with a suspension containing cell-permeable functionalized nanoparticles with bioactive molecules in the presence or absence of a magnetic field.

The use of a magnetic field in case of superparamagnetic nanoparticles renders an important increase in the contact surface area between the cells and nanoparticles and thereby ensuring improved penetration of functionalized nanoparticles through the cell membrane. Importantly, similar to poly(ethylene glycol) PEG-mediated protection of several protein-based drugs (PEG-GCSF, Amgen, Calif.; PEG-Interferon, Schering-Plough/Merck, NJ) to which PEG is attached, the nanoparticles used in conjunction with coupled peptides increase the size of the polypeptide and masks the protein's surface, thereby reducing protein degradation by proteolytic enzymes and resulting in higher gene editing efficiency.

The cells are suspended in culture medium, and non-incorporated nanoparticles can be removed by centrifugation for 10 minutes at approximately 1200×g, leaving cells that are present as clusters in the pellet. The clustered cells are then resuspended, washed again using a similar procedure and recultured in fresh medium for a suitable period. The cells can be taken through multiple cycles of separating by cell cloning or serial dilutions, resuspending, and reculturing in a culture media until a consequent biological effect triggered by the specific bioactive molecules delivered intracellularly is observed. It must be noted here that the Cas9 nuclease creates DSBs at its target site and the use of two different target sites in PD-1 gene ensures deletion of the PD-1 gene coding sequence with subsequent non-homologous end joining (NHEJ) repair that will result in knock-out of the PD-1 gene.

To confirm deletion of the PD-1 gene, the resultant clones are expanded and PCR is performed using genomic DNA from the cells and PD-1 specific primers for evaluation by electrophoresis on agarose gel and/or sequencing across the targeted sequence. The lack of appropriate fragment size will indicate successful knock-out of PD-1 gene. The newly generated human T-cells lacking PD-1 gene with acquired improved immunoresponsiveness can be further expanded and used for various purposes.

### Example 14

### Inactivating PD-1 Gene Using Insertional Mutagenesis by Non-Integrating Functionalized Nanoparticles

### Prophetic

This Example discloses non-integrating functionalized nanoparticles for inactivating a PD-1 gene via insertional mutagenesis.

The PD-1 gene functions via its interaction with its ligands PD-L1 or PD-L2. Hence, introducing a pre-mature stop codon within exon 1 of PD-1 will result in loss of PD-1 function in target T-cells and a significantly improved immune response. To knock-in a premature stop codon, the functionalized nanoparticles are prepared as described herein except that a nickase generating a SSB instead of Cas9 (that creates a DSB) is used along with gRNA with homology to the target sequence in exon 1 of PD-1 gene (two pairs of nanoparticles each with a nickase and different target-specific gRNA). These non-integrating functionalized nanoparticles will be capable of penetrating through cell membrane and reaching the nucleus where nuclease will generate an SSB at two adjacent sites in exon 1 resulting in excision of the DNA fragment in between.

In the presence of a donor template sequence with homology to the 5′ and 3′ flanking regions of the nicked sites a homologous recombination will take place resulting in insertion of the donor sequence with a stop codon in frame with the normal PD-1 coding sequence. To this end, a second type of cell-penetrating nanoparticle is generated by covalent attachment of modified donor DNA to LC2 site of the nanoparticle using specific procedure described herein.

To modify DNA for linkage to LC2 of the nanoparticle, the donor DNA fragment will be labeled at the 5′ end with ATPgamma-S(using commercial end-labeling DNA kit from Vector Labs, Burlingame, Calif.). The resultant modified donor DNA is suitable for subsequent covalent binding to the maleimide group of LC2 linker on the nanoparticle to be carried out as described for LC2 step elsewhere herein. The type II nanoparticle with donor DNA sequence is added to the cells along with the type I nanoparticle functionalized with nickase and gRNAs and the cells are cultured and clones expanded as described in Example 13. The clones of cells with PD-1 gene containing a premature stop codon in exon 1 are validated by PCR and agarose gel electrophoresis with PD-1 specific primers and/or by sequencing across the region of interest.

The methodologies described above can be used with nucleases and nickases, as well as with numerous DNA/RNA modifying enzymes for targeted gene editing and regulating target gene expression.

While certain embodiments are illustrated and described herein, it will be appreciated that various changes can be made therein without departing from the spirit and scope of the disclosure. The foregoing embodiments are therefore to be considered illustrative rather than limiting of the disclosure described herein. The scope of the disclosure is thus indicated by the appended claims rather than by the foregoing description, and all changes that come within meaning and range of equivalency of the claims are intended to be embraced herein.

1.-188. (canceled)

189. A functionalized nanoparticle for promoting the differentiation of a cell into an induced pluripotent stem cell (iPSC), said functionalized nanoparticle comprising:
(a) a nanoparticle core;
(b) a polymer coating or lipid bilayer that encapsulates the nanoparticle and having first and second functional groups that are associated with and/or attached directly thereto
(c) first and second crosslinking agents each having first and second functional groups, said first crosslinking agent having a first length and said second crosslinking agent having a second length, wherein said first crosslinking agent is attached directly to the polymer coating or lipid bilayer via a first functional group on said polymer coating or lipid bilayer and a first functional group on said first crosslinking agent and wherein said second crosslinking agent is attached directly to the polymer coating or lipid bilayer via a second functional group on said polymer coating or lipid bilayer and a first functional group on said second crosslinking agent;
(d) one or more cell targeting molecule(s); and
(e) one or more biologically active molecule(s) wherein one or more of said biologically active molecule(s) is a stem cell inducing agent or a nucleic acid encoding a stem cell inducing agent;
wherein one or more of said cell targeting molecule(s) is indirectly attached to the polymer coating or lipid bilayer via a second functional group on the first crosslinking agent and one or more of said biologically active molecule(s) is indirectly attached to the polymer coating or lipid bilayer via a second functional group on the second crosslinking agent.

190. The functionalized nanoparticle of claim 189 wherein said nanoparticle core has a hydrodynamic diameter of from 0.5 nm to 200 nm, or from 1 nm to 100 nm, or from 2 nm to 50 nm, or from 3 nm to 25 nm, or about 0.5 nm, or about 1 nm, or about 1.5 nm, or about 2 nm, or about 2.5 nm, or about 3 nm, or about 3.5 nm, or about 4 nm, or about 4.5 nm, or about 5 nm, or about 10 nm, or about 15 nm, or about 20 nm.

191. The functionalized nanoparticle of claim 189 wherein said nanoparticle core comprises a metal selected from the group consisting of iron and gold.

192. The functionalized nanoparticle of claim 189 wherein said polymer coating or lipid bilayer (1) reduces nanoparticle cytotoxicity, (2) increases nanoparticle hydrophilicity or hydrophobicity, and/or (3) provides a surface that can be modified with one or more functional groups for attachment to one or more crosslinking agents, biologically active molecules, and/or cell targeting molecules.

193. The functionalized nanoparticle of claim 189 wherein one or more of said functional groups is selected from the group consisting of amino groups (—NH₂), sulfhydryl groups (—SH), carboxyl groups (—COOH), guanidyl groups (—NH₂—C(NH)—NH₂), hydroxyl groups (—OH), azido groups (—N₃), and carbohydrates.

194. The functionalized nanoparticle of claim 189 wherein one or more of said functional groups comprises a stabilizing group that is selected from the group consisting of phosphate, diphosphate, carboxylate, polyphosphate, thiophosphate, phosphonate, thiophosphonate, sulphate, sulphonate, mercapto, silanetriol, trialkoxysilane-containing polyalkylene glycol, polyethylene glycol, a carbohydrate, and a phosphate-containing nucleotide.

195. The functionalized nanoparticle of claim 189 wherein one or more of said cross-linking agents is selected from the group consisting of long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pypridyldithio)-proprionate (SPDP); long-chain N-Succinimidyl-3-(pypridyldithio)-proprionate (LC-SPDP); sulfo-N-Succinimidyl-3-(pypridyldithio)-proprionate (sulfo-SPDP); long-chain sulfo-N-Succinimidyl-3-(pypridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pypridyldithio)-proprionate polyethylene glycolₘ (SPDP(PEG)ₘ); and sulfo-N-Succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

196. The functionalized nanoparticle of claim 189 wherein one or more of said cell targeting molecules is a cell membrane-penetrating molecule.

197. The functionalized nanoparticle of claim 196 wherein said cell membrane-penetrating molecule comprises five to nine basic amino acids.

198. The functionalized nanoparticle of claim 196 wherein said cell membrane-penetrating molecule comprises five to nine contiguous basic amino acids.

199. The functionalized nanoparticle of claim 189 wherein each of said stem cell inducing agents is selected from the group consisting of Lin28, Nanog, Mir-302bcad/367, Mir-302, Mir-200c, Mir-369, Oct4, Sox2, Klf4, and c-Myc, or a functional or domain or structural variant thereof.

200. The functionalized nanoparticle of claim 189 wherein two, three, four, five, or more of said one or more biologically active molecule(s) are stem cell inducing factors each of which is independently selected from the group consisting of Lin28, Nanog, Mir-302bcad/367, Mir-302, Mir-200c, Mir-369, Oct4, Sox2, Klf4, and c-Myc, or a functional domain or structural variant thereof.

201. The functionalized nanoparticle of claim 189 wherein said nanoparticle core has a diameter of from 0.5 nm to 50 nm.

202. The functionalized nanoparticle of claim 189 wherein said nanoparticle core comprises a metal selected from the group consisting of iron and gold.

203. A method for manufacturing a functionalized nanoparticle for promoting the differentiation of a cell into an induced pluripotent stem cell (iPSC), said method comprising attaching a stem cell inducing agent and a cell targeting molecule to a nanoparticle core.

204.-208. (canceled)

209. The method for manufacturing a functionalized nanoparticle for promoting the differentiation of a cell into an induced pluripotent stem cell (iPSC) of claim 203 wherein said stem cell inducing agent and said cell targeting molecule are independently attached to said nanoparticle core via a cross-linking agent that is selected from the group consisting of long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pypridyldithio)-proprionate (SPDP); long-chain N-Succinimidyl-3-(pypridyldithio)-proprionate (LC-SPDP); sulfo-N-Succinimidyl-3-(pypridyldithio)-proprionate (sulfo-SPDP); long-chain sulfo-N-Succinimidyl-3-(pypridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pypridyldithio)-proprionate polyethylene glycolₘ (SPDP(PEG)ₘ); and sulfo-N-Succinimidyl-3-(pypridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

210. (canceled)

211. The method for manufacturing a functionalized nanoparticle for promoting the differentiation of a cell into an induced pluripotent stem cell (iPSC) of claim 203 wherein said stem cell inducing agent is selected from the group consisting of Lin28, Nanog, Mir-302bcad/367, Mir-302, Mir-200c, Mir-369, Oct4, Sox2, Klf4, and c-Myc, or a functional domain or structural variant thereof to a nanoparticle core.

212. A method for promoting the differentiation of a cell into an induced pluripotent stem cell (iPSC), comprising contacting the cell with a functionalized nanoparticle comprising a nanoparticle core to which a stem cell inducing agent and a cell targeting molecule are attached.

213.-217. (canceled)

218. The method for promoting the differentiation of a cell into an induced pluripotent stem cell (iPSC) of claim 212 wherein said stem cell inducing agent and said cell targeting molecule are independently attached to said nanoparticle core via a cross-linking agent that is selected from the group consisting of long-chain succinimidyl 4-(N-maleimidomethyl) cyclohexane-1-carboxylate (LC-SMCC); sulfosuccinimidyl 4-(N-maleimidomethyl) cyclohexane-1-carboxylate (sulfo-SMCC); long-chain sulfosuccinimidyl 4-(N-maleimidomethyl) cyclohexane-1-carboxylate (sulfo-LC-SMCC); N-Succinimidyl-3-(pyridyldithio)-proprionate (SPDP); long-chain N-Succinimidyl-3-(pyridyldithio)-proprionate (LC-SPDP); sulfo-N-Succinimidyl-3-(pyridyldithio)-proprionate (sulfo-SPDP); long-chain sulfo-N-Succinimidyl-3-(pyridyldithio)-proprionate (sulfo-LC-SPDP); 1-Ethyl Hydrochloride-3-(3-Dimethylaminopropyl) carbodiimide (EDC); long-chain 1-Ethyl Hydrochloride-3-(3-Dimethylaminopropyl) carbodiimide (LC-EDC); succinimidyl 4-(N-maleimidomethyl) polyethylene glycolₙ (SM(PEG)ₙ); sulfosuccinimidyl 4-(N-maleimidomethyl) polyethylene glycolₙ (sulfo-SM(PEG)ₙ); N-Succinimidyl-3-(pyridyldithio)-proprionate polyethylene glycolₘ (SPDP(PEG)ₘ); and sulfo-N-Succinimidyl-3-(pyridyldithio)-proprionate polyethylene glycolₘ (sulfo-SPDP(PEG)ₘ), where n can be from about one glycol unit to about 24 glycol units, such as about one, two, three, four, five, six, seven, eight, nine, 10, 11, 12, 13, 14, 15, 16, 17, 18, 19, 20, 21, 22, 23, or 24 glycol units and where m can be from about one glycol unit to about 12 glycol units, such as about one, two, three, four, five, six, seven, eight, nine, 10, 11, or 12 glycol units.

219. (canceled)

220. The method for promoting the differentiation of a cell into an induced pluripotent stem cell (iPSC) of claim 212 wherein said stem cell inducing agent is selected from the group consisting of Lin28, Nanog, Mir-302bcad/367, Mir-302, Mir-200c, Mir-369, Oct4, Sox2, Klf4, and c-Myc, or a functional domain or structural variant thereof to a nanoparticle core.

221.-403. (canceled)
